(19) United States
(12) Patent Application Publication
(10) Pub. No.: US 2021/0093758 A1
(43) Pub. Date: Apr. 1, 2021

(54) BLOOD COLLECTION AND INFUSION CONTAINER FOR AUTOTRANSFUSION

(71) Applicant: HACKENSACK MERIDIAN HEALTH, INC., Edison, NJ (US)

(72) Inventor: MARCO CORRALES, LONG BRANCH, NJ (US)

(73) Assignee: HACKENSACK MERIDIAN HEALTH, INC.

(21) Appl. No.: 17/037,868

(22) Filed: Sep. 30, 2020

Related U.S. Application Data
(60) Provisional application No. 62/908,326, filed on Sep. 30, 2019.

(51) Int. Cl. A61M 1/02; A61M 1/00; A61M 39/28; A61M 39/20; A61M 39/10; A61M 1/36

(52) U.S. Cl. CPC A61M 1/02; A61M 1/0007; A61M 1/0013; A61M 1/0021; A61M 2039/0036; A61M 39/20; A61M 39/10; A61M 1/3653; A61M 39/28

(57) ABSTRACT

The described invention provides a blood collection and autotransfusion container, which provides fast, efficient, safe blood collection and blood or blood products transfusion.

### CROSS-REFERENCE TO RELATED APPLICATIONS

This application claims the benefit of priority to U.S. Provisional Application No. 62/908,326, filed Sep. 30, 2019, the contents of which are incorporated by reference in their entirety.

### FIELD OF INVENTION

The described invention relates to a system, method, device, and kit related to a blood collection and infusion container for autotransfusion of blood to a subject in need thereof.

### BACKGROUND

Approximately 5 million people die every year around the world from accidental and non-accidental trauma, making trauma the leading cause of death in people under the age of 45 and the third leading cause of death overall. Exsanguination accounts for approximately one-third of these deaths, and the majority of exsanguination deaths occur within the first 48 hours of injury. Massive transfusions following hemorrhagic blood loss is reported in trauma settings, but also in cardiovascular surgery, critical care, cardiology, obstetrics, and general surgery settings (Hooper, Nicholas. “Hemorrhagic Shock.” StatPearls [Internet], U.S. National Library of Medicine, 6 May 2019, www.ncbi.nlm.nih.gov/books/NBK470382/).

Hemorrhage carries with it not only the threat of immediate death due to blood loss, but also increased mortality due to multi-organ failure and sepsis. Significant morbidity is also associated with massive blood loss. Massive blood loss often starts as a cascade of shock, inflammation, and coagulopathy that can worsen blood loss and foil attempts at resuscitation (Kabaroff, Alison, and Alison Kabaroff “Stop the Bleeding.” Journal of Emergency Medical Services, 7 Nov. 2013, www.jems.com/articles/2013/11/stop-bleeding.html).

Blunt or penetrating trauma is the most common cause of hemorrhage, followed by upper and lower gastrointestinal sources of bleeding. Obstetrical, vascular, iatrogenic, and even urological sources of bleeding have all been described. Bleeding may be internal or external. A substantial amount of blood loss to the point of hemodynamic compromise may occur in the chest, abdomen, retroperitoneum, and/or limbs. For example, the thigh itself can hold up to 1 L to 2 L of blood. While localizing and controlling the source of bleeding is of utmost importance to the treatment of hemorrhagic shock, it is also essential to replace the depletion of transvascular volume through blood loss by the transfusion of blood (Hooper, Nicholas. “Hemorrhagic Shock.” StatPearls [Internet], U.S. National Library of Medicine, 6 May 2019, www.ncbi.nlm.nih.gov/books/NBK470382/).

As patients age, physiological reserves decrease, the likelihood of anticoagulant use increases, and the number of comorbidities increase. Due to this, elderly patients are less likely to handle the physiological stresses of hemorrhagic shock and may decompensate more quickly (Id).

### Hemorrhagic Shock

Hemorrhagic shock results from the depletion of intravascular volume through blood loss to the point of being unable to match the tissues demand for oxygen. As a result, mitochondria are no longer able to sustain aerobic metabolism for the production of oxygen and switch to the less efficient anaerobic metabolism to meet the cellular demand for adenosine triphosphate. In the latter process, pyruvate is produced and converted to lactic acid to regenerate nicotinamide adenine dinucleotide (NAD+) to maintain some degree of cellular respiration in the absence of oxygen (Id).

The body compensates for volume loss by increasing heart rate and contractility, followed by baroreceptor activation resulting in sympathetic nervous system activation and peripheral vasoconstriction. Typically, there is a slight increase in the diastolic blood pressure with narrowing of the pulse pressure. As diastolic ventricular filling continues to decline and cardiac output decreases, systolic blood pressure drops (Id).

Due to sympathetic nervous system activation, blood is diverted away from noncritical organs and tissues to preserve blood supply to vital organs such as the heart and brain. While prolonging heart and brain function, this also leads to other tissues being further deprived of oxygen causing more lactic acid production and worsening acidosis. This worsening acidosis along with hypoxemia, if left uncorrected, eventually causes the loss of peripheral vasoconstriction, worsening hemodynamic compromise, and death (Id).

The body's compensation mechanisms are affected by cardiopulmonary comorbidities, age, and vasoactive medications. Due to these factors, heart rate and blood pressure responses are extremely variable and, therefore, cannot be relied upon as the sole means of diagnosis (Id).

A key factor in the pathophysiology of hemorrhagic shock is the development of trauma-induced coagulopathy which is the sum of two distinct processes: acute coagulopathy of trauma and resuscitation-induced coagulopathy. Trauma-induced coagulopathy is acutely worsened by the presence of acidosis and hypothermia. The activity of coagulation factors, fibrinogen depletion, and platelet quantity are all adversely affected by acidosis. Hypothermia (less than 34° C.) compounds coagulopathy by impairing coagulation and is an independent risk factor for death in hemorrhagic shock (Id).

The American College of Surgeons Advanced Trauma Life Support (ATLS) hemorrhagic shock classification links the amount of blood loss to expected physiologic responses in a healthy 70 kg patient. As total circulating blood volume accounts for approximately 7% of total body weight, this equals approximately five liters in the average 70 kg (or approximately 154 lbs) male patient. The table below outlines the varying classes of hemorrhagic shock (Table adapted from Id).

TABLE 1
Classes of Hemorrhagic shock

| Class | Volume |
|---|---|
| Class 1 | Volume loss up to 15% of total blood volume, approximately 750 mL. |
| | Heart rate is minimally elevated or normal. Typically, there is no change in |
| | blood pressure, pulse pressure, or respiratory rate. |
| Class 2 | Volume loss from 15% to 30% of total blood volume, from 750 mL to |
| | 1500 mL. Heart rate and respiratory rate become elevated (100 BPM to 120 |
| | BPM, 20 RR to 24 RR). Pulse pressure begins to narrow, but systolic blood |
| | pressure may be unchanged to slightly decreased. |
| Class 3 | Volume loss from 30% to 40% of total blood volume, from 1500 mL to |
| | 2000 mL. A significant drop in blood pressure and changes in mental status |
| | occur. Heart rate and respiratory rate are significantly elevated (more than |
| | 120 BPM). Urine output declines. Capillary refill is delayed. |
| Class 4 | Volume loss over 40% of total blood volume. Hypotension with narrow |
| | pulse pressure (less than 25 mmHg). Tachycardia becomes more |
| | pronounced (more than 120 BPM), and mental status becomes increasingly |
| | altered. Urine output is minimal or absent. Capillary refill is delayed. |

However, when assessing patients, clinical factors must be taken into account. For example, beta blockers, a.k.a. beta adrenergic blocking agents which reduce blood pressure by blocking the effects of the hormone epinephrine, can alter the patient's physiologic response to decreased blood volume by inhibiting the mechanism to increase heart rate. In addition, patients with baseline hypertension may be functionally hypotensive with a systolic blood pressure of 110 mmHg (Id).

Hemorrhagic shock may be treated through the administration of donated blood and/or blood products under the management strategy of “damage control resuscitation.” The concept of damage control resuscitation focuses on permissive hypotension, hemostatic resuscitation, and hemorrhage control to adequately treat the “lethal triad” of coagulopathy, acidosis, and hypothermia that occurs in trauma (Id).

### Resuscitation

Hypotensive resuscitation, meaning a resuscitative approach to the administration of IV fluids and blood with the goal of keeping mean arterial pressure high enough for essential tissue perfusion, but lower than normal to limit bleeding out, has been suggested for the hemorrhagic shock patient without head trauma (see Jeremy B. Smith, Jean-Francois Pittet, and Albert Pierce. “Hypotensive Resuscitation.” SpringerLink, Springer US, 9 May 2014, link.springer.com/article/10.1007/s40140-014-0064-7). The aim is to achieve a systolic blood pressure of 90 mmHg in order maintain tissue perfusion without inducing re-bleeding from recently clotted vessels. The term “permissive hypotension” refers to a means of restricting fluid administration until hemorrhage is controlled while accepting a short period of suboptimal end-organ perfusion. Studies regarding permissive hypotension have yielded conflicting results and must take into account the type of injury (penetrating versus blunt), the likelihood of intracranial injury, the severity of the injury, as well as proximity to a trauma center and definitive hemorrhage control (Id).

The quantity, type of fluids to be used, and endpoints of resuscitation remain topics of much study and debate. For crystalloid resuscitation, normal saline and lactated Ringer's solution are the most commonly used fluids. Normal saline has the drawback of causing a non-anion gap hyperchloremic metabolic acidosis due to the high chloride content, while lactated Ringer's can cause a metabolic alkalosis as lactate metabolism regenerates into bicarbonate (Id).

Recent trends in damage control resuscitation focus on “hemostatic resuscitation” which pushes for early use of blood products rather than an abundance of crystalloids in order to minimalize the metabolic derangement, resuscitation-induced coagulopathy, and the hemodilution that occurs with crystalloid resuscitation. The end goal of resuscitation and the ratios of blood products remain at the center of much study and debate. A recent study has shown no significant difference in mortality at 24 hours or 30 days between ratios of 1:1:1 and 1:1:2 of plasma to platelets to packed RBCs. However, patients that received the more balanced ratio of 1:1:1 were less likely to die as a result of exsanguination in 24 hours and were more likely to achieve hemostasis. Additionally, reduction in time to first plasma transfusion has shown a significant reduction in mortality in damage control resuscitation. Id.

In addition to blood products, and antifibrinolytics (meaning products that prevent the breakdown of fibrin in clots) have been studied for their utility in the treatment of hemorrhagic shock in the trauma patient. Several antifibrinolytics have been shown to be safe and effective in elective surgery. Research has shown that in a randomized control trial of tranexamic acid versus placebo in trauma, tranexamic acid has been shown to decrease overall mortality when given in the first eight hours of injury. Follow-up analysis showed additional benefit to tranexamic acid when given in the first three hours after surgery (Id).

### Allogeneic Blood Transfusions

Allogeneic blood (blood from unrelated/anonymous donors) has a number of concerns and risks. Approximately 15 million blood and blood component transfusions occur annually within the United States, making it a common lifesaving and life-sustaining intervention, especially within intensive care, surgical, and trauma units. Various estimates indicate that between 15% and 45% of patients require transfusions during their intensive care unit (ICU) stay, depending on their length of stay. A blood transfusion is, in essence, a liquid transplant of living tissue, and transfusing blood is a time-consuming, high-risk nursing activity. When implementing a blood transfusion, the nurse's primary focus should be on patient safety and the 3 “rights”: right blood component, right patient, and right time. Therefore, blood transfusion protocols reinforce the need for redundant checks of blood component type to blood order and procured blood product to patient's identification. These checks play a critical role in minimizing the risk of transfusion reactions. Protocols also stress time. Blood transfusion should begin within 30 minutes of blood pickup from the lab and be completed within 4 hours of removal from a controlled temperature storage environment in order to minimize blood degradation and microbial contamination. The latter concern is based on data related to the “lag phase” that occurs before bacteria begin to proliferate following removal from refrigeration. Researchers in a variety of recent, controlled studies have reported a direct relationship between the liberal use of allogeneic blood products in a patient and the resulting increase in serious complications, such as increased nosocomial infection rates, ventilator times, ICU and hospital lengths of stay, autoimmune diseases, and cancer recurrence rates. Many hospitals are now implementing restrictive transfusion strategies (using a hemoglobin threshold of 7 g/dL vs 10 g/dL). With the exception of patients with active coronary ischemic syndromes, restrictive blood transfusions have resulted in decreased morbidity and mortality rates. It is believed that these blood transfusion risks are related to aging blood storage effects and to the immune-modulating effects that occur to some degree in all blood transfusion recipients (Kessler, Christine. “Priming Blood Transfusion Tubing: A Critical Review of the Blood Transfusion Process.” Critical Care Nurse, 1 Jun. 2013, ccn.aacnjournals.org/content/33/3/80. short).

Because whole blood is rarely used in allogeneic blood transfusions, blood components/products, such as the components/products in the table below, are commonly added for transfusions and come with a host of their own protocols and thus capital and labor intensive expenditures (see below table, taken from Kessler, Christine. “Priming Blood Transfusion Tubing: A Critical Review of the Blood Transfusion Process.” Critical Care Nurse, 1 Jun. 2013, ccn. aacnj ournals. org/content/33/3/80.short).

TABLE 2
Blood Components or Blood Products

| Component | Features |
|---|---|
| Red blood cells | Prepared from whole blood by removal of most of the plasma; shelf |
| | life of 35-42 days; stored at a temperature of 4° C. ± 2° C. |
| | Volume runs between 180 and 350 ml and is usually infused within |
| | 90-120 minutes; may be infused in 5-10 minutes if warranted |
| | On average, 1 unit raises hemoglobin level by 1 g/dL (or 2-3%) |
| Platelets | Derived from whole blood; stored at 20-24° C. in plastic containers (in |
| | motion), shelflife of 5 days |
| | Each unit can increase platelet count by 5-10 × 10^{/9}/L; beneficial in |
| | severe thrombocytopenia |
| | Does not require ABO cross-matching; although ABO type-specific |
| | platelets should be provided whenever possible |
| | Volume is 180-400 mL depending on whether the platelets were |
| | derived by apheresis (special filtration) or are pooled, equivalent to 6 |
| | random donor platelet units; can be infused within 30 minutes |
| Fresh frozen plasma | Stored at temperature of −30° C.; has a frozen shelf life of 24 months |
| | Must be thawed to 1-6° C. before use, which takes approximately 15- |
| | 30 minutes; should be infused within 4 hours of thawing |
| | Has a volume between 240 and 300 mL; usually transfused at |
| | approximately 30 minutes per unit |
| | Contains multiple coagulation factors; used when factor deficiency |
| | (and threat hemorrhage) occurs in liver disease, disseminated |
| | intravascular coagulation, and for urgent reversal of warfarin |
| | anticoagulation |
| Cryoprecipitate | Like fresh frozen plasma, cryoprecipitate is stored frozen with a shelf |
| | life of 24 months |
| | Must be thawed under controlled condition, which takes 15-30 minutes |
| | Once thawed, must be transfused within 4 hours |
| | Volume of 100-200 mL (each unit equal to 5 donor pools), which can |
| | raise fibrinogen levels by about 1 g/L |
| | Generally used for patients with von Willebrand disease or severe |
| | hypofibrinemia who are at risk of hemorrhage |
| Granulocytes | Can be stored for only 24 hours |
| | Volume is variable; typically administered in 1-2 hours |
| | A unit of granulocytes contains red blood cells so needs to be ABO- |
| | type specific; a large number of lymphocytes are also present, making |
| | febrile reactions common; usually irradiated to help minimize |
| | reactions |
| | Mainly given to neutropenic cancer patients with bacterial sepsis that |
| | is unresponsive to conventional antibiotic therapy |

Possible adverse reactions associated with allogeneic blood transfusions are provided in the table below (Below table, taken from Kessler, Christine. “Priming Blood Transfusion Tubing: A Critical Review of the Blood Transfusion Process.” Critical Care Nurse, 1 Jun. 2013, ccn.aacnjournals.org/content/33/3/80. short).

TABLE 3
Adverse reactions

| Reaction | Description |
|---|---|
| Allergic Reaction | Results from an interaction of an allergen in the transfused blood with |
| | antibodies in the blood recipient. Occasionally, infusion of antibodies |
| | from the donor may be involved. Reactions can range from skin |
| | irritation to more serious symptoms such as difficulty breathing. |
| Acute hemolytic | Occurs when a patient is given an incompatible blood type and occurs |
| transfusion reaction | during, immediately after, or within 24 hours of a transfusion. The |
| | recipient's body immediately begins to destroy the donated red blood |
| | cells, resulting in fever, pain, and sometimes kidney failure. |
| Delayed hemolytic | A much milder version of acute hemolytic transfusion reaction that |
| transfusion reaction | occurs between 24 hours and 28 days after a transfusion. Symptoms |
| | are usually milder than in acute hemolytic transfusion reactions and |
| | may even be absent. |
| Febrile nonhemolytic | The most common transfusion reaction, characterized by fever and/or |
| transfusion reaction | chills in the absence of hemolysis. May occur during or up to 4 hours |
| | after a transfusion. These reactions are generally mild and respond |
| | quickly to treatment, but fever indicates a more severe reaction with |
| | more serious causes. |
| Hypotensive transfusion | A sudden decrease in systolic blood pressure may occur soon after a |
| reaction | transfusion begins and is quickly resolved by stopping the transfusion |
| | and delivery of fluid volume. |
| Post-transfusion purpura | A rare but potentially fatal condition that may occur when a |
| | transfusion recipient develops antibodies against donor platelets, |
| | resulting in destruction of both transfused platelets and the patient's |
| | own platelets. Can cause a severe decline in the platelet count; |
| | usually occurs 5-12 days after a transfusion. Women are more |
| | affected than men. |
| Transfusion-associated | Occurs when the volume of blood or blood components are |
| circulatory overload | transfused and cannot be effectively processed by the recipient. Can |
| (TACO) | be due to an excessively high infusion rate and/or volume. Patients |
| | with heart or kidney disorders are at greatest risk. |
| Transfusion-related | A rare but serious reaction that occurs when fluid builds up in the |
| acute lung injury | lung within 6 hours of transfusion, but is not related to the volume of |
| (TRALI) | blood or blood products infused. Symptoms include inexplicable, |
| | profound acute respiratory distress. Cause is not well understood, but |
| | reaction is thought to be associated with antibodies in donor blood. |

Initial concerns about allogeneic transfusions arose from the transmission of viral infection including the hepatitis viruses, human immunodeficiency virus (HIV) and human T-cell lymphotropic viruses (HTLV). Rigorous and reliable screening tests have reduced the risk of infection from transfusion, but have added to the cost of each unit. More recently, concerns have focused on blood-borne transmission of variant Creutzfeldt-Jakob disease (vCJD) when case reports emerged of presumed transmission of vCJD via allogenic blood transfusion. Unlike hepatitis and HIV, there is no effective screening test and the disease has a variable and often prolonged asymptomatic incubation period. Leukodepletion was introduced to further reduce the risk of vCJD transmission via blood transfusion, further adding to the cost of allogeneic transfusions and the need for additional blood products. As a result, allogenic donor blood is becoming an increasingly costly and scarce resource. As demand for blood is outstripping donation, there is a real social and economic pressure to increase the proportion of blood transfused by autologous transfusion (Walunj, et al. “Autologous Blood Transfusion.” OUP Academic, Oxford University Press, 24 Aug. 2006, academic.oup.com/bjaed/article/6/5/192/337094).

When ordering transfusions, typically medical staff describe the amount of blood being transfused in “units.” The term “unit” is defined in the literature in several different ways. First, it can describe the volume of blood or RBCs transfused as measured by milliliters (mL) or by cubic centimeters (cc); examples in the literature describe one unit of blood transfused as 250-500 cc, which converts to 250-500 mL (see Elzik, Mark E., et al. “Correlation of Transfusion Volume to Change in Hematocrit—Elzik—2006—American Journal of Hematology—Wiley Online Library.” American Journal of Hematology, John Wiley & Sons, Ltd, 23 Jan. 2006, onlinelibrary.wiley.com/doi/epdf/10.1002/ajh.20517). Second, it can describe a 2.3% to 10.5% average increase in a patient's hematocrit per liter of blood or RBCs transfused, where a patient's hematocrit is the ratio of the volume of blood cells to the total volume of blood (see id).

In non-traumatic situations, blood is usually transfused at a rate no faster than 25 to 50 units per hour for the first 15 minutes in order to assess for adverse reactions. Vital signs, which should include respiratory rate, are measured before the infusion and again every 15 minutes for the first 30 minutes, then hourly until completion of the transfusion. A follow-up check is recommended an hour after the transfusion. More frequent measurement of vital signs may be necessary with rapid transfusions. Through surveillance studies, it is now recognized that as many as 34% of reactions occur more than 30 minutes after the transfusion starts and sometimes many hours after its conclusion. It is therefore prudent to observe patients during the subsequent 24 hours.

Blood transfusion rate is vastly different where a patient's body is going through hemorrhagic shock and requires a massive blood transfusion. The traditional definition of a massive blood transfusion is 20 units RBCs in 24 hours, which corresponds to approximately 1 blood volume in a 70 kg patient. A commonly used definition in the trauma literature is >10 units RBCs in 24 hours. While both of these definitions are reasonable for research, they are not practical in an ongoing resuscitation. In hospital settings, other definitions of a massive blood transfusion include: loss of 0.5 blood volume within 3 hours; use of 50 units of blood components in 24 hours; use of 6 units RBCs in 12 hours. From a practical standpoint, requirement for >4 RBC units in 1 hour with ongoing need for transfusion, or blood loss >150 ml/min with hemodynamic instability and need for transfusion are reasonable definitions in the setting of a massive blood transfusion situation (see, below table for typical blood volume; see also Patil, Vijaya, and Madhavi Shetmahajan. “Massive Transfusion and Massive Transfusion Protocol.” Indian Journal of Anaesthesia, Medknow Publications & Media Pvt Ltd, September 2014, www.ncbi.nlm.nih.gov/pmc/articles/PMC4260305/).

Typical blood volume is dependent on age, height and weight of a patient, as shown in Table 4 below.

TABLE 4
Classification and range of circulating
blood volumes in children and adults

| Classification | Blood volume (ml/kg) |
|---|---|
| Neonates (birth to 1 month) | 85-90 |
| Infant (1 month to 2 years) | 75-80 |
| Children | 70-75 |
| Adults | 65-70 |

The equipment used in the transfusion of blood components includes filters, tubing (straight or “Y” tubing), needles, pumps, blood warmers, and rapid infusion devices. The transfusion of all blood components requires the use of an inline filter to remove debris in the form of blood clots and blood cell aggregates. “Standard” blood administration sets contain a plastic mesh filter designed to remove most particles, large cell aggregates, and fibrinous material. Most standard transfusion sets have filters with pore size ranging from 170 μm to 260 μm, which is adequate to filter most blood components. Special white-blood-cell filters are no longer required, because all blood components are now leukocyte-depleted by blood banks and laboratories. Leukocyte-reduced and irradiated blood reduce immunologic response complications and decrease the risk of graft versus host rejection (Id).

The size of the peripheral venous cannula for transfusing blood to a patient in need depends on the size and integrity of the vein and the speed at which the blood component must be transfused. A larger, 18-gauge catheter is preferred as it provides good flow rates for blood components without causing excessive discomfort to the patient. Smaller catheters, as small as 24 gauge, may be used in pediatric or adult patients with small, inaccessible veins which require slower flow rates (Id).

Blood transfusion tubing should not be used for more than 4 hours, but it can be used to transfuse multiple, consecutive units of blood of the same tissue type and compatibility. Most institutions limit transfusions to just 2 (or as much as 4) units per filter and limit the duration of tubing use to 4 hours, whichever comes first.

Specialized transfusion equipment, such as pressure bags and blood warmers, is used depending on the patient's condition and should be managed in accordance with the manufacturer's guidelines. Pressure bags are helpful when blood must be transfused rapidly (about 5 minutes per unit) and should be inflated only until blood flow through the drip chamber is continuous (about 200 mm Hg). Blood warmers are used when there is a risk of serious cardiac arrhythmia due to rapid infusion of large volumes of cold blood cells (>6 units/h) or when transfusing blood into a patient who has high blood levels of cold agglutinins (or a history of cold urticaria). Strong clinical evidence supports this practice. Blood should be warmed to 37° C. and should never exceed 42° C. or the manufacturer's recommendations. Platelets, fresh frozen plasma, and cryoprecipitate do not require warming

It is simpler and far more desirable to collect and retransfuse the subject's own blood as needed, in a process known as autologous blood transfer. A main method of autologous blood transfusion is known as cell salvage, where blood is collected via suction and retransfused back to the patient after filtration or washing, and warmed if necessary. Cell-salvage can be performed in an emergency setting, intraoperatively, and postoperatively. It is widely practiced in emergency situations in and out of medical settings, cardiothoracic, vascular, orthopedic, neuro, and transplantation surgery, as well as in post-operative intensive care settings. The use of a patient's own blood reduces the risk of transmission of infection, transfusion reaction, provides safer transfusion in scenarios with rare blood groups and multiple auto-antibodies, contains near normal oxygen carrying capacity and near normal pH, eliminates immunosuppression, increases acceptability to patient groups that require special care (such as Jehovah Witness patients), reduces the demand on allogeneic blood supplies, and provides costs savings (Id).

Various devices have been developed to drain and collect fluids, for example in U.S. Pat. No. 3,853,128. However, such devices do not have the capability of auto-infusing simultaneously with draining. As a result, there are significant time-consuming intervening steps between the collection of the blood, rendering the blood appropriate for transfusion, and the transfusion thereof. As discussed en supra, treatment of blood loss and blood loss morbidity and/or mortality is dependent on volume of blood lost. The more time that passes means that more blood is lost, and thus a higher likelihood of related injury or mortality. Therefore, it desired that the device is able to collect blood and transfuse it back to the patient quickly.

Furthermore, other prior art blood collection devices are not able to collect blood from certain locations on a subject's body, specifically, pleural spaces, because of their inability to regulate pressure in the drainage system. Pressure fluctuations result when fluid exits the collection chamber to be retransfused into a patient, the remaining fluid volume drops and pressure negativity increases. If pressure negativity is not maintained within a narrow window, then excess bleeding, damage to the intrathoracic tissue, and/or exsanguination may occur. Therefore, it is desired that the device is able to collect blood and transfuse it back to the patient safely.

### Chest/Drainage Tubes

When a patient's chest or pleural cavity fills with fluid and/or air, a medical health practitioner must first insert a drainage tube or a chest tube in order to drain it in a procedure called a tube thoracostomy. Tube thoracostomy is a common procedure in which a tube or small catheter is placed through the chest wall into the pleural cavity and used primarily to drain air or fluid, but the tube can also be used to instill agents to induce pleurodesis (a procedure that uses medication to adhere the lung to the chest wall, which seals up the space between the outer lining of the lung and chest wall (pleural cavity) to prevent fluid or air from continually building up around the lung) or to treat empyema (collection of pus, e.g., in the pleural cavity) (see Huggins, John, et al. “Placement and Management of Thoracostomy Tubes and Catheters in Adults and Children.” UpToDate, Wolters Kluwer, July 2019, https://www.uptodate.com/contents/placement-and-management-of-thoracostomy-tubes-and-catheters-in-adults-and-children). Thoracostomy tube placement may be indicated for the following conditions: Pneumothorax, such as a spontaneous pneumothorax, traumatic pneumothorax (including occult pneumothorax [ie, pneumothorax identified on computed tomography]), Iatrogenic pneumothorax (most commonly due to central line placement), tension pneumothorax, extension of mediastinal air, air leak following pulmonary resection, bronchopleural fistula, postoperative or due to mechanical ventilation; hemothorax, such as from chest trauma (blunt or penetrating), postoperative following thoracic or upper abdominal surgery, nontraumatic cardiac or aortic conditions; pleural effusion, such as, sterile effusion, infected or inflammatory effusion (ie, empyema, parapneumonic effusion), malignant effusion, chylothorax, other effusions from diaphragmatic injury in adults, esophagus rupture and biliary fistulas; and pleurodesis for the treatment of refractory effusion (see id).

However, care should be taken for patients with the following contraindications: anticoagulation, coagulopathy, overlying infection, or a bleeding diathesis are relative contraindications for emergent chest tube placement in both adults and children with the exception of acutely injured patients with concern for massive hemothorax or tension pneumothorax; needle thoracostomy may be a preferred initial option in a patient with a pneumothorax and a coagulopathy to avoid additional procedure-related bleeding; and transudative pleural effusions due to liver failure are not generally managed with thoracostomy drainage (see id). Further, in a patient with pleural adhesions, previous pleurodesis, or prior pulmonary surgery, blind insertion of a chest tube is dangerous; guidance by ultrasound or computed tomography (CT) scan without contrast is preferred (see id).

Generally, a single tube in inserted into the side of a patient's chest in order to accomplish drainage. Multiple tubes may be inserted when a single tube is not accomplishing its task. The chest tube insertion site depends upon the indication (fluid or air, and extent and location of accumulation) and patient.

Chest tubes are typically latex rubber, silicone, or polyvinyl chloride (PVC) tubes. Alternative devices (such as Blake drain 19 French, 24 French) can also serve as thoracostomy drains, if necessary. Chest tubes are categorized by their sizes as measured in French Gauge (Fr), a unit of measurement that refers to the outer diameter of the tube. One Fr is equivalent to 0.333 mm. Various chest tube sizes can be seen in the table below (Table modified from id). The inner diameter of the chest tube will be variable depending on the thickness of the wall of the tube, which varies according to manufacturer. However, the wall of a chest tube is typically configured to meet regulatory and non-regulatory standards, which includes biocompatible material, material that can withstand pressure, suction, temperature, and fluid flow.

TABLE 5
Chest Tube Dimensions

| French | Circumference | OD | OD |
|---|---|---|---|
| Gauge | (mm) | (mm) | (in.) |
| 3 | 3.14 | 1 | 0.039 |
| 4 | 4.19 | 1.333 | 0.053 |
| 5 | 5.24 | 1.667 | 0.066 |
| 6 | 6.28 | 2 | 0.079 |
| 7 | 7.33 | 2.333 | 0.092 |
| 8 | 8.34 | 2.667 | 0.105 |
| 9 | 9.42 | 3 | 0.118 |
| 10 | 10.47 | 3.333 | 0.131 |
| 11 | 11.52 | 3.667 | 0.144 |
| 12 | 12.57 | 4 | 0.158 |
| 13 | 13.61 | 4.333 | 0.17 |
| 14 | 14.66 | 4.667 | 0.184 |
| 15 | 15.71 | 5 | 0.197 |
| 16 | 16.76 | 5.333 | 0.21 |
| 17 | 17.81 | 5.667 | 0.223 |
| 18 | 18.85 | 6 | 0.236 |
| 19 | 19.9 | 6.333 | 0.249 |
| 20 | 20.94 | 6.667 | 0.263 |
| 22 | 23.04 | 7.333 | 0.288 |
| 24 | 25.13 | 8 | 0.315 |
| 26 | 27.23 | 8.667 | 0.341 |
| 28 | 29.32 | 9.333 | 0.367 |
| 30 | 31.42 | 10 | 0.393 |
| 32 | 33.51 | 10.667 | 0.419 |
| 34 | 35.6 | 11.333 | 0.445 |

A medical health practitioner will select a chest tube size dependent on patient age, patient weight, accumulated substance, and patient need (i.e., class of hemorrhagic shock). The table below outlines some factors that go into choosing chest tube size (Table modified from id).

TABLE 6
Chest Tube Size Selection.

| Patient age | Patient weight | | Air | Serous fluid | Pus | Blood |
|---|---|---|---|---|---|---|
| Neonate/newbom | <11 lbs | (<5 kg) | 8 to 14 Fr | 8 to 14 Fr | 8 to 14 Fr | 14 to 20 Fr |
| Infant/child | 11 to 22 lbs | (5 to 10 kg) | 8 to 14 Fr | 8 to 14 Fr | 12 to 18 Fr | 18 to 24 Fr |
| | 22 to 33 lbs | (10 to 15 kg) | 8 to 14 Fr | 8 to 14 Fr | 12 to 18 Fr | 18 to 24 Fr |
| | 33 to 44 lbs | (15 to 20 kg) | 8 to 14 Fr | 8 to 14 Fr | 18 to 24 Fr | 18 to 24 Fr |
| | 44 to 66 lbs | (20 to 30 kg) | 8 to 14 Fr | 8 to 14 Fr | 18 to 24 Fr | 18 to 24 Fr |
| Preteen/teen/adult | >66 lbs | (>30 kg) | 8 to 24 Fr | 8 to 24 Fr | 24 to 32 Fr | 24 to 36 Fr |

### Chest Drainage Devices

Intrapleural pressure, meaning the pressure within the pleural cavity, is always negative, which acts like a suction to keep the lung inflated. The negative intrapleural pressure is due to the surface tension of the alveolar fluid, the elasticity of the lungs, and the elasticity of the thoracic wall. The surface tension of the alveolar fluid tends to pull parts of the lungs inward, therefore pulling the entire lung inward. This pull causes negative pressure. Further, the elasticity of the tissue in the lungs tends to recoil and pull the lung inward. As the lung moves away from the thoracic wall, the cavity becomes slightly larger, acting as a suction and enhancing negative pressure in the pleural cavity. Lastly, the elastic thoracic wall tends to pull away from the lung, further enlarging the pleural cavity and adding to the negative pressure in the pleural cavity.

When a patient's pleural cavity fills with fluid, such as blood, and air, it causes the ordinarily negative pressure of the pleural cavity to turn positive, thus preventing lung function. To drain the pleural space of a patient, a device is required that a) removes fluid and air as quickly as possible, b) prevents drained air and fluid from returning to the pleural space, and c) restores the negative pressure in the pleural space to restore lung function. Therefore, a drainage device must i) allow air and fluid to leave the chest; and ii) contain a one way valve to prevent air and fluid from returning to the chest; and iii) have a configuration or means for drainage either via suction or gravity.

In its most basic configuration, a chest drainage system has an airtight water seal/drainage chamber, a tube that connects to the chest tube in the patient, through the airtight water seal/drainage chamber, and extends 2-5 cm below the water seal level in the chamber. (See FIG. 1A). In order to decompress the positive pressure, this system contains a secondary tube that begins above the water seal level through the airtight seal and out of the water seal/drainage chamber to vent into the atmosphere. When pleural pressure is positive in a subject, the pressure in the patient tube becomes positive, and if the positive pressure in the patient tube is greater than the depth to which the tube is immersed in the water seal, then the air and fluids will enter into the bottle, which will be depressurized by the vent into the atmosphere. If the pleural pressure is negative, it will move liquid from the water seal into the patient tube, and air will not enter the pleural cavity or the rigid straw. This system is called the water seal system because the water bottle seals the pleural cavity from the air or liquid outside of the body.

If large quantities of fluid need to be collected, an additional drainage chamber can be added to the chest drainage system. (See FIG. 1B). The drainage theory is similar to a one-bottle system, but allows the fluid drained from the patient to be collected into a separate drainage chamber, and a third tube connecting the drainage chamber and water seal chamber allows the positive pressure to flow from the patient's pleural cavity, through the drainage chamber, into the water seal chamber and out the vent. When suction drainage is used instead of gravity drainage, a third chamber that regulates suction control is added to the chest drainage system. (See FIG. 1C). Chest drainage units are then translated from the three chamber system for simple, convenient and disposable hospital use. (See FIG. 1D).

Chest drainage devices are well known in the art, and include ALTITUDE™ Dry Suction Chest Drainage Unit (Cardinal Health®), AQUA-SEAL™ Chest Drainage Unit (Cardinal Health®), ALTITUDE™ Dry Suction Chest Drainage Unit (Cardinal Health®), ARGYLE™ One and Two Bottle Chest Drainage Systems (Cardinal Health®), ARGYLE™ THOR-SEAL™ (I, II, III) Chest Drainage Unit (Cardinal Health®), SENTINEL SEAL™ Chest Drainage Unit (Cardinal Health®), SAHARA® S-1100 Series, PLEUR-EVAC® Chest Drainage System (Teleflex®), and the like.

### Blood Collection Devices

An additional problem, specifically in the situations where the patient is susceptible to exsanguination, is the ability to rapidly transfuse the blood back to the patient. Some devices solve this problem by providing a device that forces the blood back to the patient or to a blood transfusion bag for transfusion with a blood transfusion device. For example, some devices contain a compressible mechanical device inside the blood drainage and collection chamber, and through the manual pumping action of the compressible device, the blood can be forced out of the drainage/collection chamber and back into the patient or to a blood transfusion bag for transfusion with a blood transfusion device. However, the use of a compressible device inside of the blood collection chamber may require capital expenditure, such as increased use of specific disposable materials or such as increased staff training and use of time to learn how to use devices additional to the technology hospitals already have. Furthermore, additional mechanics being in contact with the collected blood puts the patient at risk for damage to the cells, leaching of metals and/or plastics into the collected blood, and/or the potential for the mechanics themselves to malfunction or break-off into the collected blood.

As discussed en supra, the rapid and safe transfusion of blood is essential for patients at risk for Class 2 and above hemorrhagic shock. While generally it is possible to give a patient blood through the force of gravity, however, this approach is not medically feasible in situations where rapid transfusion is needed. In some hospitals, the delivery of blood and/or blood products is through the use of a device, such as a pump or a compressor, which uses mechanical and/or electro-mechanical forces to rapidly force the blood back into the patient. One example of such a device is an infusion pump. However, a common concern is whether the infusion pump mechanism will injure the cells being transfused, specifically red blood cell hemolysis. Therefore, the compression and/or pump mechanism must be safe for the transfer of living cells. Another concern is the transfusion of unwarmed blood, where the transfusion of unwarmed blood can cause hypothermia which can exacerbate a patient's risk of morbidity and/or mortality. Thus, a common component of blood transfusion systems is the use of a blood warmer. In this context, the warming of blood, and generally the transfer of blood, comes with the risk of the formation of air bubbles. Therefore, another common component of blood transfusion systems is the use of a gas vent to release the air bubbles prior to transfusing to the patient. Yet another concern is the possibility of foreign materials, such as particles or bacteria, present in the blood, and clots or clumps of biological materials that may accumulate in the blood. For this reason, blood transfusion systems often contain a blood filter. Filter size may vary according to desired particle level of filtration. A representative figure that describes the flow of blood can be seen in FIG. 2A. Instead of having several separate devices, hospitals may have a singular blood transfusion device that contains all the described components and rapidly and safely transfuses blood to a patient (see FIG. 2B).

Automatic blood transfusion devices are well known in the art and include Level 1® H-1200 Fast Flow Fluid Warmer series (SMITHS MEDICAL), the Belmont® Rapid Infuser R series (Belmont Medical Technologies), and the like.

It is essential that the construction of a blood collection container be safe and durable for immediate use of the collected blood and be compatible with blood drainage devices and with blood transfer devices for many reasons. First, because retransfusion of a patient's blood must be performed in the shortest amount of time possible, extensive filtering of the blood is not an option. Second, because the blood is the patient's own blood, the blood contains all the components necessary to be life-saving. Thus, extensive filtration and the addition of blood products are unnecessary. Third, the blood collection bag needs to be able to hold living cells with minimal damage to the cells. Therefore, the blood collection bag must be sterile, suitable for living cells, suitable for the contents inside to undergo minimal processing, and capable of rapidly collecting blood from a body cavity and rapidly transfusing the collected blood to the patient.

Therefore, it is desired that the device be able to rapidly collect blood and rapidly transfuse it back to the patient quickly, safely, efficiently, and cost-effectively.

Despite advances in transfusion medicine and medical care, massive blood loss is still a major cause of mortality around the world. It is therefore an object of the present invention to provide a quick and efficient system, method, device, and kit related to an empty blood collection and infusion container for the drainage and autotransfusion of blood.

### SUMMARY OF THE INVENTION

According to one aspect, the described invention provides a blood collection and autotransfusion container device for rapid collection and transfusion of blood or blood products, comprising: a blood container, an infusion member, and a transfusion member; wherein the infusion member is connected to the blood container and the infusion member is configured to be in fluid communication with a subcutaneous drainage site on the subject and the blood container; and wherein the transfusion member is connected to the blood container and the transfusion member is configured to be in fluid communication with the blood container and a subcutaneous transfusion site on the subject; wherein the blood container is configured with the following characteristics: a volume of about 25 mL, about 30 mL, about 35 mL, about 40 mL, about 45 mL, about 50 mL, about 80 mL, about 85 mL, about 90 mL, about 95 mL, about 100 mL, about 200 mL, about 450 mL, about 500 mL, about 600 mL, about 700 mL, about 800 mL, about 900 mL, about 1000 mL, about 1100 mL, or about 1200 mL, inclusive, a length between about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, and about 40 cm, inclusive, a width between about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, and about 30 cm, inclusive, and is made of biocompatible, deformable, compressible material; wherein the infusion member has an internal diameter between about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm, inclusive; wherein the transfusion member has an internal diameter between about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm, inclusive; and wherein the blood collection and autotransfusion container is effective to collect and transfuse one unit of blood in less than about 10 minutes, in less than about 9 minutes, in less than about 8 minutes, less than about 7 minutes, less than about 6 minutes, less than about 5 minutes, less than about 4 minutes, or less than about 3 minutes.

According to one embodiment, the blood collection and autotransfusion container device further comprises one or more tubing fittings configured to connect to and be in fluid communication with the blood container, infusion member or transfusion member, wherein the one or more tubing fitting comprises one or more port members, one or more tube members, one or more membrane member, one or more connector/coupler members, one or more filter members, one or more valve members, and/or one or more penetrating members, and/or any combination thereof.

According to another embodiment of the device, the blood collection and autotransfusion container device further comprises one or more tubing accessories configured to attach to the blood container, infusion member or transfusion member, wherein the tubing accessories comprises one or more hanging members, one or more cap members, and/or one or more clamp members and/or any combination thereof.

According to another embodiment, the infusion member comprises one or more tubing fittings comprising a port member and a membrane member, and one or more tubing accessories comprising a cap member; or one or more tubing fittings comprising a tube member and a penetrating member, and one or more tubing accessories comprising a cap member and a clamp member.

According to another embodiment, the transfusion member comprises one or more tubing fittings comprising a port member and a membrane member, and one or more tubing accessories comprising a cap member; or one or more tubing fittings comprising a tube member and a penetrating member, and one or more tubing accessories comprising a cap member and a clamp member.

According to another aspect, the described invention provides a method for rapidly collecting accumulated blood from a subject in need thereof and rapidly transfusing blood or blood products to the subject in need thereof comprising. a) providing a blood collection and autotransfusion container device comprising: a blood container device, an infusion member, and a transfusion member; wherein the infusion member is connected to the blood container and the infusion member is configured to be in fluid communication with a subcutaneous drainage site on the subject and the blood container; and wherein the transfusion member is connected to the blood container and the transfusion member is configured to be in fluid communication with the blood container and a subcutaneous transfusion site on the subject; b) draining accumulated blood from the body cavity of a subject wherein the blood collection and autotransfusion container is connected to the subject via the infusion member; c) collecting the blood in the blood collection and autotransfusion container by channeling the blood through the infusion member to a blood collection compartment in the blood container; and d) transfusing the collected blood back to the subject by connecting the blood collection and autotransfusion container to the subcutaneous access site of the subject via the transfusion member; wherein the transfusion member is configured to be in fluid communication with a subcutaneous transfusion site on the subject and the blood collection compartment in the blood container; and wherein the blood collection and autotransfusion container is effective to collect and transfuse one unit of blood in less than about 10 minutes, in less than about 9 minutes, in less than about 8 minutes, less than about 7 minutes, less than about 6 minutes, less than about 5 minutes, less than about 4 minutes, or less than about 3 minutes.

According to one embodiment of the method, the step of providing a blood collection and autotransfusion container further comprises providing one or more tubing fittings configured to connect to and be in fluid communication with the blood container, infusion member or transfusion member; and providing one or more tubing accessories configured to attach to the blood container, infusion member or transfusion member; wherein the one or more tubing fitting comprises one or more port members, one or more tube members, one or more membrane member, one or more connector/coupler members, one or more filter members, one or more valve members, and/or one or more penetrating members, and/or any combination thereof; and wherein the one or more tubing accessories comprises one or more hanging members, one or more cap members, and/or one or more clamp members and/or any combination thereof.

According to another embodiment, the step of draining accumulated blood from the body cavity of a subject comprises forcing the accumulated blood to be drained from a subject wherein the force comprises a gravitational force or a suction force; wherein forcing the accumulated blood to be drained from a subject by a gravitational force comprises configuring the spatial arrangement of the blood collection and autotransfusion container to be at a lower height than the body cavity of the subject; wherein forcing the accumulated blood to be drained from a subject by a suction force comprises connecting a suction tube with suction action to the blood container of the blood collection and autotransfusion container and activating the suction action; or optionally providing a chest drainage device and wherein forcing the accumulated blood to be drained from a subject by a gravitational force comprises configuring the spatial arrangement of the chest drainage device to be at a lower height than the body cavity of the subject and the blood container of the blood collection and autotransfusion container to be at a lower height than the chest drainage device; or optionally providing a chest drainage device wherein forcing the accumulated blood to be drained from a subject by a suction force comprises connecting a suction tube with suction action to the chest drainage device and activating the suction action.

According to another embodiment, the step of collecting the blood comprises connecting the subcutaneous drainage site to the infusion member of the blood collection and autotransfusion container and forcing the blood to be channeled to the blood collection and autotransfusion container wherein the force comprises a gravitational force; wherein forcing the blood to be channeled to the blood collection and autotransfusion container by gravitational force comprises arranging the spatial configuration of the blood container of the blood collection and autotransfusion device to be at a lower height than the blood.

According to another embodiment, the step of transfusing the collected blood back to the subject comprises forcing the blood to be transfused to the subject through the transfusion member wherein the force comprises a compression force, gravitational force, or by a pressurized pump; wherein forcing the blood to be transfused to the subject through the transfusion member by a compression force comprises providing a compression chamber attaching the blood container of the blood collection and autotransfusion container in the compression chamber with compression action and activating the compression action; wherein forcing the blood to be transfused to the subject through the transfusion member by a gravitational force comprises configuring the spatial arrangement of the blood container to be at a higher height than the subcutaneous transfusion site on the subject; or wherein forcing the blood to be transfused to the subject through the transfusion member by a pressurized pump comprises providing a pressurized pump and attaching the pressurized pump to the blood container of the blood collection and autotransfusion container activating the pressurizing action.

According to another embodiment, the step of transfusing the collected blood back to the subject further comprises: filtering the collected blood by connecting a blood filter; warming the collected blood by connecting a blood warmer; and releasing the excess gas in the collected blood by connecting to a gas vent; wherein each of the blood filter, the blood warmer and gas vent is in fluid communication with the transfusion member and the subcutaneous transfusion site on the subject.

### BRIEF DESCRIPTION OF DRAWINGS

FIG. 1A shows an example of a one-bottle chest drainage system comprising an airtight water seal/drainage chamber, a first tube that connects to a chest tube in a patient, through the airtight water seal/drainage chamber where the first tube extends 2-5 cm below the water seal level in the chamber, and a second tube that begins above the water seal level through the airtight seal and out of the chamber to vent into the atmosphere; where the second tube decompresses the positive pressure, this system contains a secondary tube .

FIG. 1B shows an example of a two-bottle chest drainage system, which comprises all the elements of the one-bottle system, plus a second drainage chamber to collect the drained fluid, and a third tube to connect the drainage chamber to the water seal chamber, thereby allowing the system to decompress through the vent.

FIG. 1C shows an example of a three-bottle chest drainage system which comprises all the elements of the two-bottle system shown in FIG. 1B, plus a third bottle, which acts as a pressure regulator for when suction power is used to drain the fluid from a patient.

FIG. 1D shows an example of a commercialized disposable chest drainage unit, comprising the elements of a three-bottle chest drainage system.

FIG. 2A is a diagrammatic representation that describes the various paths to transfuse blood or blood products to a patient. For example, blood or blood products can be directly transfused from a transfusion bag to the patient through the force of gravity (a). In another example, blood or blood products can be transfused from the transfusion bag through the force of compression or pump pressure to the patient (b). In another example, blood or blood products can be transfused from the transfusion bag through the force of compression or pump pressure through a filter to the patient (c). In another example, blood or blood products can be transfused from the transfusion bag through the force of compression or pump pressure, through a filter, and through a gas vent to the patient (d). In another example, blood or blood products can be transfused from the transfusion bag through the force of compression or pump pressure, through a filter, through a gas vent, and through a warmer to the patient (e). In another example, blood or blood products can be transfused from the transfusion bag to the patient through the force of gravity through a filter to the patient (f). In another example, blood or blood products can be transfused from the transfusion bag to the patient through the force of gravity, through a gas vent to the patient (g). In another example, blood or blood products can be transfused from the transfusion bag to the patient through the force of gravity, through a warmer to the patient (h).

FIG. 2B shows an example of a commercialized blood transfusion device as used in health care centers that comprises the elements as described in FIG. 2A, i.e., a transfusion bag, a pressure chamber, a filter, a gas vent, a warmer, as well as the ability to connect the transfusion bag to any combination of the described elements.

FIG. 3A shows a cross-section front view of a first representation of the blood collection and autotransfusion container of one embodiment of the described invention.

FIG. 3B shows a perspective view of a first embodiment of the blood collection and autotransfusion container of the described invention.

FIG. 3C shows a cross-section front view of a second embodiment of the blood collection and autotransfusion container of the described invention.

FIG. 3D shows a perspective view of a second embodiment of the blood collection and autotransfusion container of the described invention.

FIG. 4A shows front and side views of tubing fittings 5a, 5b, 5c, 5c, 5d, 5e, 5f, and 5g that can be used with embodiments of the described invention.

FIG. 4B shows front and side views of tubing accessories 6a, 6b, and 6c that can be used with embodiments of the described invention.

FIG. 5 shows a side view of an example of the connections between a patient, a chest drainage unit, the blood collection and autotransfusion container of the described invention, and an autotransfuser device.

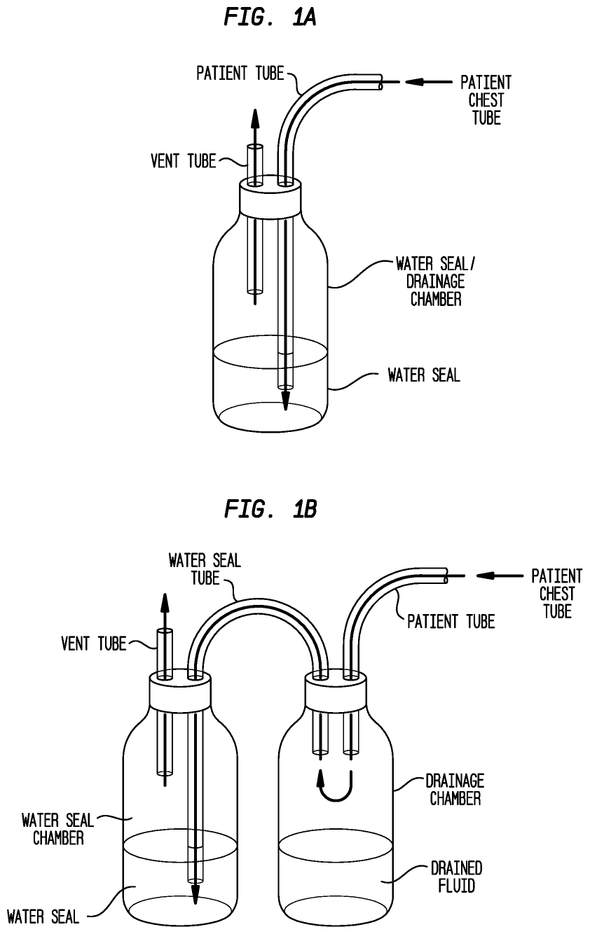

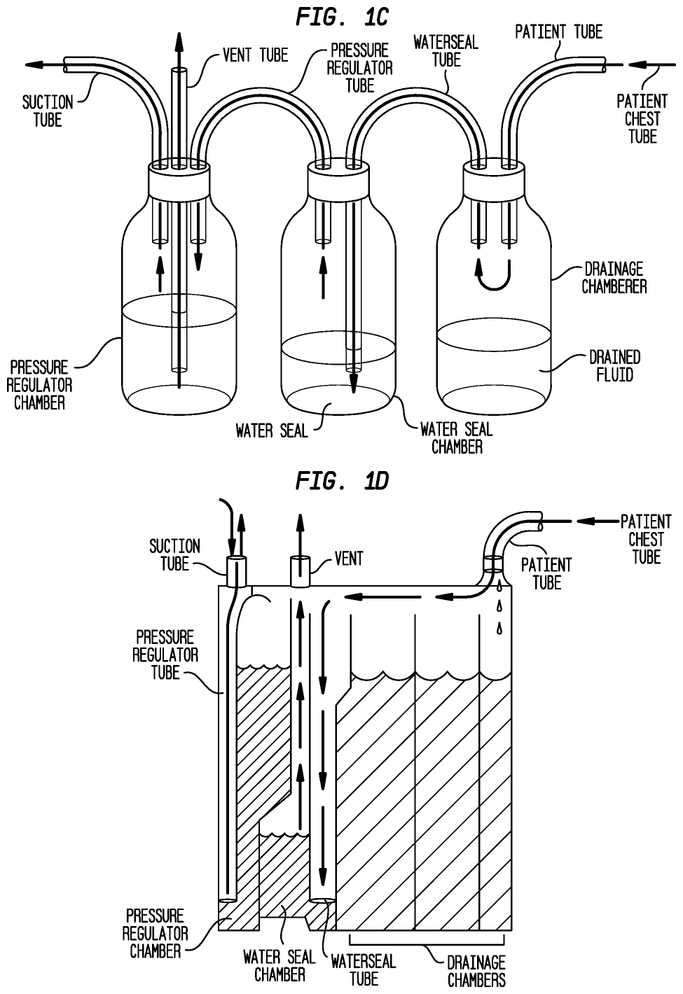

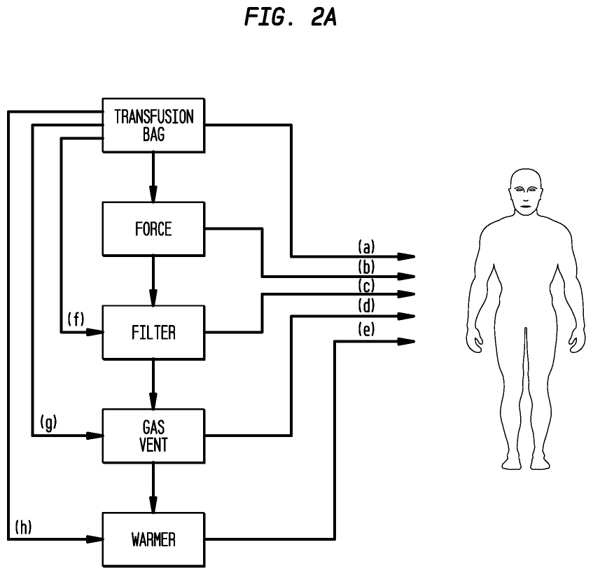

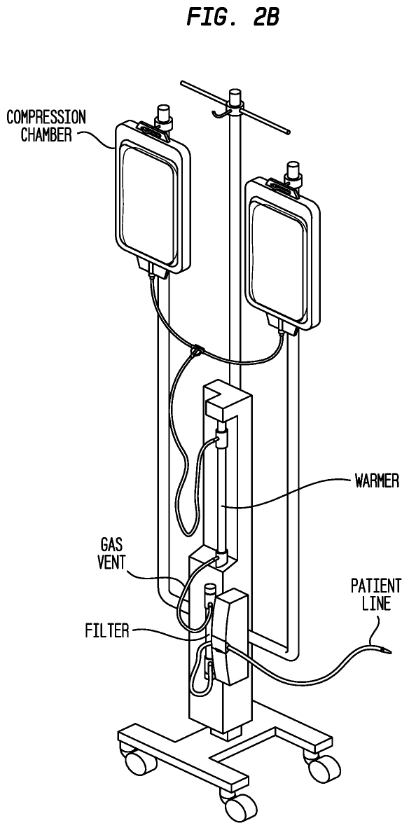

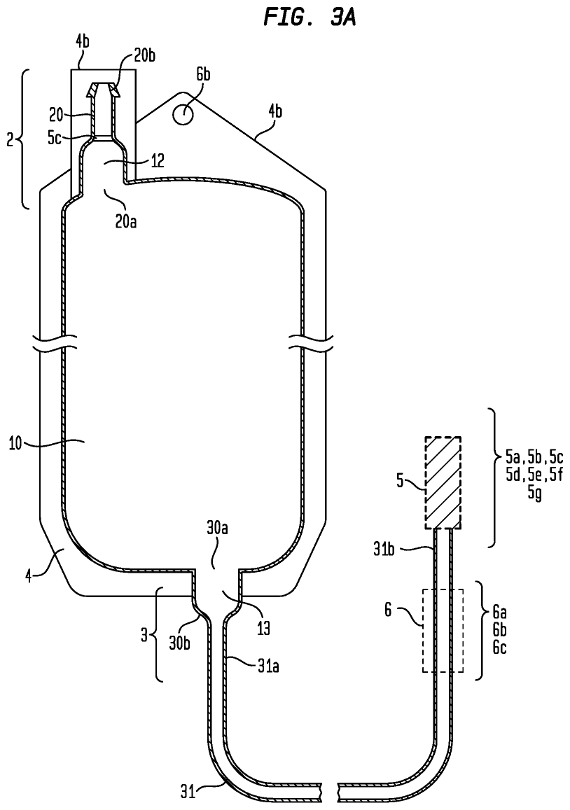

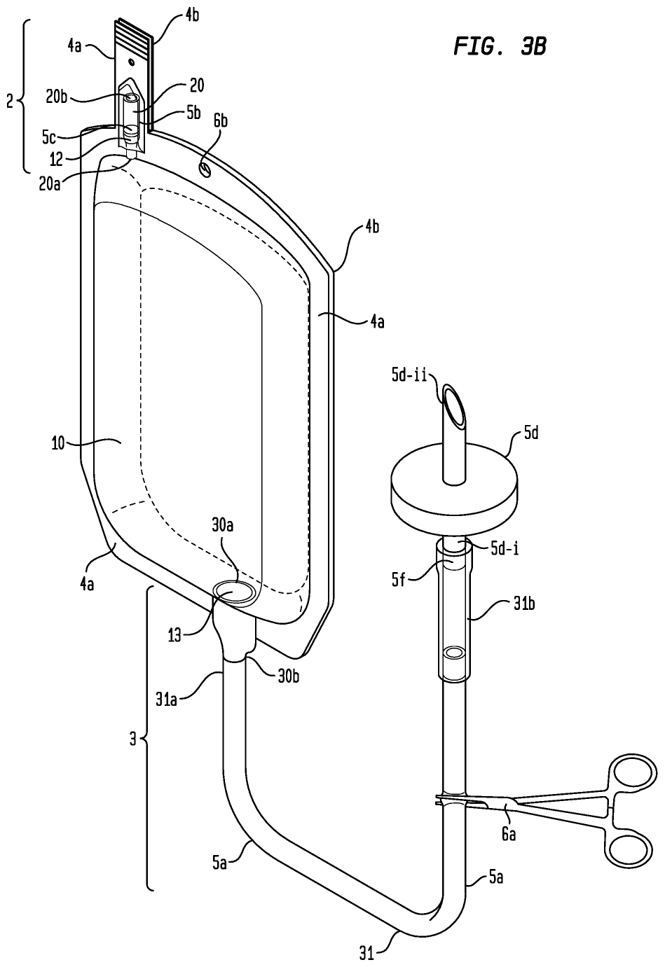

### DETAILED DESCRIPTION

### Definitions

The term “anti-coagulant agent” as used herein means an agent that slows or inhibits coagulation of blood

The term “antimicrobial agent” as used herein refers to any substance that kills or slows the growth of microbes.

The term “anti-thrombotic agent” as used herein refers to an agent that reduces formation of blood clots.

The term “attached” as used herein, along with all of its grammatical variations thereof, including but not limited to “attach,” “attachment,” “attaching” “attachable,” “attachably,” is used to describe the state of an object being fastened onto a second object; or the ability and/or capability of an object to be fastened onto a second object; through mechanical, frictional, electrostatic, and/or electromagnetic forces.

The term “autotransfuser” as used herein interchangeable with “automatic blood transfuser” is used to describe a device that automatically transfuses blood to a patient. An autotransfuser as used herein comprises one or more compression chambers; optionally a blood warmer; optionally a blood filter; and optionally a gas vent each of the foregoing terms are described herein.

The term “cannula” as used herein is used to describe a thin hollow pointed tube, typically made from biocompatible metals or biocompatible plastic, for insertion into a subject's body.

The term “catheter” is used to describe a tube, typically made from biocompatible plastic that is inserted into a subject's body and is configured to allow fluid communication.

The term “clamp” is used herein to describe a first object or instrument that attaches to the structure of a second object and is configured to compress the structure to regulate or occlude flow of material, such as fluid or blood, in that structure.

The term “clip” is used herein to describe a first object that attaches to a structure of a second object and is configured to attach the second object to a third structure or object.

The term “coagulation” and its various grammatical forms as used herein refers to a process of forming blood a clot (of blood). Coagulation involves a series of zymogen activation reactions. At each stage, a precursor protein, or zymogen, is converted to an active protease by cleavage of one or more peptide bonds in the precursor molecule. The components that can be involved at each stage include a protease from the preceding stage, a zymogen, a nonenzymatic protein cofactor, calcium ions, and an organizing surface that is provided by the damaged blood vessel and platelets in vivo. The final protease to be generated is thrombin (factor IIa).

The protease zymogens involved in coagulation include factors II (prothrombin), VII, IX, X, XI, XII, and prekallikrein. Factors V and VIII are homologous 350,000 dalton proteins. Factor VIII circulates in plasma bound to von Willebrand factor, while factor V is present both free in plasma and as a component of platelets. Thrombin cleaves V and VIII to yield activated factors (Va and VIIIa) that have at least 50 times the coagulant activity of the precursor forms. Factors Va and VIIIa have no enzymatic activity themselves, but serve as cofactors that increase the proteolytic efficiency of Xa and IXa, respectively. Tissue factor (TF) is a nonenzymatic lipoprotein cofactor that greatly increases the proteolytic efficiency of VIIa. It is present on the surface of cells that are not normally in contact with blood and plasma (e.g. fibroblasts and smooth muscle cells) since they are abluminal from the endothelium. TF is a key factor that initiates coagulation outside a broken blood vessel.

In vivo, two pathways of coagulation are recognized: the intrinsic coagulation pathway so called because all of the components are intrinsic to plasma, and an extrinsic coagulation pathway (tissue factor (TF) pathway), which generates a thrombin burst and is initiated when tissue thromboplastin activates Factor VII. Tissue factor exposed by tissue injury, either traumatically, by disease or surgery, can activate sufficient factors X, IX and thrombin (II) to initiate coagulation. The extrinsic and intrinsic systems converge to activate the final common pathways causing fibrin formation.

The intrinsic system (contact activation pathway) is initiated when blood contacts any surface except normal endothelial and blood cells. The intrinsic system begins with formation of the primary complex on collagen by high-molecular weight kininogen (HMWK), prekallikrein, and FXII (Hageman factor). Prekallikrein is converted to kallikrein and Factor XII becomes Factor XIIa. Factor XIIa converts Factor XI into Factor XIa. Factor XIa activates Factor IX, which, with its co-factor Factor VIIIa form the tenase complex, which activates Factor X to Factor Xa.

Fibrinogen is a 330,000 dalton protein that consists of three pairs of polypeptide chains (designated α, β and γ) covalently linked by disulfide bonds. Thrombin converts fibrinogen to fibrin monomers (Factor IA) by cleaving fibrinopeptides A (16 amino acid residues) and B (14 amino acid residues) from the amino-terminal ends of the α and β chains respectively. Removal of the fibrinopeptides allows the fibrin monomers to form a gel. Initially, the fibrin monomers are bound to each other noncovalently. Subsequently, factor XIIIa catalyzes an interchain transglutamination reaction that cross-links adjacent fibrin monomers to enhance the strength of the clot.

Fibrin participates in both the activation of Factor XIII by thrombin and activation of plasminogen activator (t-PA),It specifically binds the activated coagulation factors factor Xa and thrombin and entraps them in the network of fibers, thus functioning as a temporary inhibitor of these enzymes which stay active and can be released during fibrinolysis . . . .

As used herein the term “component” is used interchangeably with the terms “element,” “part,” “thing,” “item,” or “structure” to describe a constituent of an object.

The term “collect” as used herein is used to refer to gathering together; assemble or accumulate.

The term “communication” or “fluid communication” as used herein is used to describe the configuration of a component or object that is efficient to allow the flow of fluid, such as blood. Fluid communication can be from a first object to a second object, from an object to a subject, or from a subject to an object.

The term “connect,” as used herein, along with all of its grammatical variations thereof, including but not limited to “connectable,” “connecting,” “connected,” “connectably,” is used to describe the state of an object being joined with a second object; or the ability and/or capability of an object being joined with a second object. According to some embodiments, the connection is efficient to allow fluid communication between the two objects. Typically, an object has two or more ends, and at least one of those ends is configured to connect to a complementary end of a second object. Examples of complementary ends include a female end and a male end, wherein the male end of a first object penetrates the female end of a second object thereby connecting the two objects. In this example, the outer dimensions of the male end must be configured to fit inside the inner dimensions of the female end. According to some embodiments, the inner dimensions of the male end and the female end are configured to allow fluid communication between the two objects. According to some embodiments, connections are configured to be mechanically sound to avoid being inadvertently disconnected. According to some embodiments, connections are configured to be airtight to prevent the leakage of fluid and/or the influx of air. According to some embodiments, connections are configured to preserve, maintain, or allow sterility of the fluid communication between the objects.

The terms “configured,” or “configuration” as used herein are used to describe an object or a component of an object that is formed in order to accomplish a function.

The terms “connector” or “coupling” as used herein describes a small fitting, typically made of plastic, which is configured to connect at least two tubes, at least three tubes, at least four tubes, at least five tubes, etc. There are several types and shapes of connectors/couplings, which include straight, elbow, tee, cross, and Y-shaped.

As used herein the term “contiguous” describes two or more components or objects that contact each other at one or more locations along their exposed surfaces.

The term “diaphragm” as used herein is used to describe a thin wall, typically circular shaped, in a structure that is configured to occlude the flow of material and is also configured to be cored or penetrated by a penetrating member, such as a needle or a spike, to allow the flow of material. The term “diaphragm” is used interchangeably with the terms “septum,” and “membrane.”

The term “drainage device” as used herein is used to describe a device that facilitates draining accumulated fluid from a subject. An exemplary drainage device comprises: a drainage tube connector that connects to a drainage tube in a subject; a pressure regulator; one or more drainage chambers; and a collection drain that channels drained blood out of the device.

The term “drainage tube” as used herein is used to describe a tube inserted into the body cavity of a subject in order to drain gas or fluids accumulated therein.

The term “elongated” as used herein is used to describe a structure wherein the length of the structure is longer than the width.

The terms “form” and “formed” as used herein means to mold, cast, cut, shape, or create an object.

The term “filter” as used herein is an object that is configured to remove contaminants or aggregated material from blood.

The term “hub” is used to describe an object that enables a penetrating member to be connected to a secondary object or component such as a tube.

The term “infusion” is used to describe the introduction of fluid, such as blood or a saline solution, into an object or component.

The term “interdigitate” as used herein describes two objects or two components of an object that are configured to interlock together.

The term “orientation” as used herein refers to a position of an object in its relation to space. When describing the autologous blood collection and autotransfusion container, the orientation of the device is such that if it is hanging, the portion pointing towards the ground is considered the bottom and the portion at 180° in the opposite direction is considered the top.

The term “lumen” as used herein is used to describe the space in the interior of a tubular structure.

The term “luer,” “luer slip” or a “luer lock” as used herein is used to describe a male or female taper on the end of a hub or syringe to connect a needle to a syringe or a second luer fitting to the first luer fitting.

The term “membrane” as used herein is used to describe a thin wall in a structure that is configured to occlude the flow of material from one cavity to another, and is also configured to be penetrated or cored by a penetrating material, such as a needle or a spike, to allow the flow of material.

The terms “microbe” or “microorganism” are used interchangeably herein to refer to a microscopic organism, meaning one too small to be seen clearly with the naked eye, including, but not limited to, bacteria, fungi (molds), algae, Archaea, protozoa, and viruses.

The term “needle” as used herein is a thin, small pointed hollow steel tube for insertion into a subject's body with a hub attached to it.

The term “occlude,” “occlusion,” “occluded,” “occluding” and any grammatical variation thereof, as used herein is used to describe the configuration of an component or object so that it obstructs fluid communication between two or more objects, components, or cavities. The occluding object or component may be “un-occluded,” or “non-occluded,” or any variation there, such as by penetration by a penetrating member or release of the object's occluding action to allow for fluid communication between the two or more objects, components, or cavities.

The term “position” as used herein refers to a condition of something with reference to its place or location.

As used herein, the term “port” is used to describe a first object or component that functions as a channel into the interior of a second object or component, or into the interior of a subject, and which is efficient for the insertion or extraction of material, such as fluid.

The term “plug” as used herein is used to describe a first object that occludes the flow of material in a second object.

The term “preservative” as used herein refers to a chemical substance used to prevent (for a reasonable time) the growth of microorganisms, or occurrence of undesirable chemical reactions.

As used herein, the terms “pressure differential” and “differential pressure” are used interchangeably herein to describe a pressure measured relative to the pressure in the atmosphere around it. “Atmospheric pressure” as used herein is used to describe the pressure exerted by the atmosphere, usually considered as the downward pressure of air onto a unit of area of the earth's surface; the unit of pressure at sea level, which is about 15 lb per square inch (2.17 kPa), is one atmosphere. “Negative pressure” as used herein is used to describe a pressure less than that of the atmosphere. “Positive pressure” as used herein is used to describe a pressure greater than that of the atmosphere.

As used herein, the term “pressure regulator,” “pressure differential regulator,” or “differential pressure regulator,” are used interchangeably herein to describe a device or an object that regulates pressure differentials by allowing for the release of positive pressure that build in a drainage device or a collection device when draining a subject of accumulated blood, and allowing for the maintenance or increase of negative pressure in a subject's chest cavity when draining the subject of the accumulated blood.

The phrase “regulation of pressure differential,” and any grammatical variation thereof, as used herein describes the decompression of positive pressure that builds up when draining accumulated air and/or fluid in a patient's chest cavity so that the fluid and/or air appropriately drains and negative pressure is restored in the patient's chest cavity.

The term “separably” as used herein describes two components or objects that are in contiguous alignment or in contact with each other that are configured to have the ability to be dissociated from each other so that they are no longer in contiguous alignment or in contact. The term “non-separably” as used herein describes components that are not able to be separated.

The term “subcutaneous drainage site” is used herein to describe a point on a subject's body or an object in a subject's body that allows for access into the inner body cavity of the subject, to drain fluids accumulated therein.

The term “subcutaneous transfusion site” is used herein to describe a point on a subject's body or an object in a subject's body that allows for the transfusion of blood into the vascular system of the subject.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human. A human may be of any age, such as neonatal, infant, child, adult, or geriatric. As used herein a “subject in need” is described as a subject that is in danger of or experiencing Class I, Class II, Class III, or Class IV hemorrhagic shock and requires a massive blood transfusion.

The term “suitable” as used herein is used to describe the structural dimensions and physical characteristics of a component or an object that still allows it to function as described. For example, a component that is suitable for fluid communication, such as the communication of blood, has structural dimensions, such as having an hollow interior, to allow the flow of fluid and is comprised of biocompatible materials, such as antimicrobial material.

The terms “sterile,” as used herein, along with all of its grammatical variations thereof, including but not limited to “sterility,” “sterilization,” and “sterilized,” describes the state or ability of being free from, or the destruction of, all microorganisms in or about an object, such as by steam (flowing or pressurized), chemical agents (alcohol, phenol, heavy metals, ethylene oxide gas), high-velocity electron bombardment, heat, or radiation. As used in this application, components are sterilized or in a sterile state with minimal impact on the integrity of the materials described herein.

The term “syringe” as used herein is used to describe a hollow cylindrical structure that is configured to hold and eject fluid out of one end.

The term “thrombosis” as used herein refer to formation of blood clots (meaning the soft, coherent, jelly-like mass resulting from the conversion of fibrinogen to fibrin, thereby entrapping the red blood cells (and other formed elements) within the coagulated plasma). Fibrin is split by plasmin, a serine protease that hydrolyzes peptide bonds. In vertebrates, blood clotting is a result of cascade regulation from fibrin.

The term “transfusion” as used herein is used to describe the transfer of blood or blood component(s) to a recipient.

The term “trauma” as used herein is used to describe an injury to the body of a subject or a patient. Traumatic injuries are generally of a sudden onset and require immediate medical attention. Typically, there are two main types of trauma: blunt force trauma, which occurs when an object or a force strikes the body, often causing concussions, deep cuts or broken bones; or penetrating trauma, which is when an object pierces the skin or body, usually creating an open wound. Trauma can occur in any number of circumstances and settings. For example, trauma can occur in uncontrolled environments, such as in motor vehicle collisions, sports injuries, falls, natural disasters, and otherwise at home, on the street, or while at work. Trauma can also occur in controlled environment, such as during surgery.

The term “truncated” as used interchangeably with “abbreviated” as used herein is used to describe a structure wherein the width of the structure is as long as or longer than the length.

As used herein, the term “tube” is a hollow cylindrical structure or canal and is configured to allow fluid communication. The term “medical tube” is used to describe the use of a tube in a medical setting. Medical tubes are typically made from biologically compatible materials that are sterilized or can be sterilized without damage to the material and without putting the biological materials at risk for damage. A medical tube can have one lumen, or it can have more than one lumen.

The term “tubing accessories” as used herein is used to describe objects attached to medical tubing or tubing fittings during medical procedures such as when administering an IV, or transfusing blood to a patient. Non limiting examples include: clamps, hooks or hangers, caps, and clips. According to some embodiments, tubing accessories comply with regulatory and non-regulatory standards, including, but not limited to, the Food and Drug Administration standards, International Standards Organization standards, U.S. Pharmacopeial Conventional Standards, Good Manufacturing Practice standards, Current Good Manufacturing Practice standards, Advancing Transfusion and Cellular Therapies Worldwide standards, and the like.

The term “tubing fittings” as used herein is used to describe objects connected to medical tubing during medical procedures, such as when administering an IV, or transfusing blood to a patient. Non limiting examples include: tubing, valves, membranes, plugs, connectors or couplings, ports, filters, spikes, needles, hubs, valves, luers, chambers, catheters, or cannulas. According to some embodiments, tubing fittings are provided with additional features to facilitate in connection with one or more components. For example, tubing fittings may be configured to have a “barb” where a barb is additional wing-like material on the exterior of the fitting. In another example, tubing fittings are configured to have a “bore” where a bore is additional cone-like material on the exterior of the fitting. In another example, tubing fittings are configured to have a “thread” where a thread is additional spiral-like material on the exterior of the fitting. According to some embodiments, the tubing fitting(s) comply with regulatory and non-regulatory standards, including, but not limited to, the Food and Drug Administration standards, International Standards Organization standards, U.S. Pharmacopeial Conventional Standards, Good Manufacturing Practice standards, Current Good Manufacturing Practice standards, Advancing Transfusion and Cellular Therapies Worldwide standards, and the like.

The term “valve” is an object that can be attached to or connected to a second object and is configured to occlude or regulate the flow of material.

The term “vascular access device” is an object that provides access to the peripheral veins or central veins of a subject.

The term “vent” as used herein describes as object that is connected to two or more objects that communicate material between them and is configured to release gas that may exist in the material being communicated.

As used herein, the term “warmer” is used to describe an object that is connected to two or more objects or components that communicate material between them, and is configured to increase the temperature of with gentle or moderate heat (“warms”) the material being communicated. For example, the warmer may be a blood warmer that is connected to two or more tube and that warms the blood flowing from a first tube to a second tube. In this embodiment, the warmer must warm in a manner that does not harm the biological materials in the blood. Warming action may be done through electromagnetic forces, heating forces, and the like.

### Blood Collection and Autotransfusion Container

According to one aspect, the described invention provides a blood collection and autotransfusion container useful for the collection of blood from a subject and the transfusion of blood or blood products to the subject.

According to some embodiments, the described invention provides a blood collection and autotransfusion container useful for the rapid collection of blood accumulating in a subject. According to some embodiments, the blood collection and autotransfusion container is configured to collect one unit of blood accumulating in a subject in less than about 9 minutes, in less than about 8 minutes, less than about 7 minutes, less than about 6 minutes, less than about 5 minutes, less than about 4 minutes, or less than about 3 minutes. For example, according to some embodiments, the blood collection and autotransfusion container is configured to collect one unit of blood accumulating in a subject in less 5 minutes.

According to some embodiments, the described invention provides a blood collection and autotransfusion container useful for the rapid transfusion of blood or blood products to a subject. According to some embodiments, the blood collection and autotransfusion container is configured for the transfusion of one unit of blood in less than about 7 minutes, in less than about 6 minutes, in less than 5 minutes, less than about 4 minutes, less than about 3 minutes, less than about 2 minutes, or less than about 1 minute. For example, according to some embodiments, the blood collection and autotransfusion container is configured for the transfusion of one unit of blood in less than about 3 minutes.

According to some embodiments, the described invention provides an blood collection and autotransfusion container configured to rapidly collect one unit of blood accumulating in a subject and to effect the transfusion of blood or blood products to the subject in in less than about 10 minutes, in less than about 9 minutes, in less than about 8 minutes, less than about 7 minutes, less than about 6 minutes, less than about 5 minutes, less than about 4 minutes, or less than about 3 minutes. For example, according to some embodiments, the blood collection and autotransfusion container described herein is configured to rapidly collect one unit of blood accumulating in a subject and to effect the transfusion of blood or blood products back to the subject in less than about 8 minutes.

According to some embodiments, a unit of blood can be determined by the capacity of the blood collection and autotransfusion container described herein. According to some embodiments, one unit of blood is between 25 mL and 2500 mL. According to some embodiments, the capacity of the blood collection and autotransfusion container and the unit of blood container therein depend on the patient population. According to some embodiments, the patient population may be neonatal, infant, child, or adult. For example, one unit of blood for a neonatal patient population is between 25 mL and 35 mL; one unit of blood for an infant patient population is between 40 mL and 50 mL; one unit of blood for a child patient population is between 80 mL and 100 mL; and one unit of blood for an adult population is between 200 mL and 1200 mL.

According to some embodiments, the blood collection and autotransfusion container described herein is configured for the collection and transfusion of more than one unit of blood. According to some embodiments, the blood collection and autotransfusion container is configured for the collection and transfusion of more than one unit of blood, more than two units of blood, more than three units of blood, more than four units of blood, more than five units of blood, more than six units of blood, more than seven units of blood, more than eight units of blood, and more than nine units of blood.

According to some embodiments, the number of units used to collect and transfuse blood is dependent on the capacity of the blood collection and autotransfusion container, the patient population, and the needs of the patient. For example, a neonatal patient may require 9 units of blood wherein one unit of blood is 25 mL. In another example, a neonatal patient may require 9 units of blood wherein one unit of blood is 30 mL. In another example, a neonatal patient may require 9 units of blood wherein one unit of blood is 35 mL. In another example, an infant patient may require 6 units of blood wherein one unit of blood is 40 mL. In another example, an infant patient may require 6 units of blood wherein one unit of blood is 45 mL. In another example, an infant patient may require 6 units of blood wherein one unit of blood is 50 mL. In another example, a child patient may require 3 units of blood wherein one unit of blood is 80 mL. In another example, a child patient may require 3 units of blood wherein one unit of blood is 85 mL. In another example, a child patient may require 3 units of blood wherein one unit of blood is 90 mL. In another example, a child patient may require 3 units of blood wherein one unit of blood is 95 mL. In another example, a child patient may require 3 units of blood wherein one unit of blood is 100 mL. In another example, an adult patient may require 5 units of blood wherein a unit of blood is 200 mL. In another example, an adult patient may require 4 units of blood wherein a unit of blood is 450 mL. In another example, an adult patient may require 3 units of blood wherein a unit of blood is 700 mL. In another example, an adult patient may require 2 units of blood wherein a unit of blood is 950 mL. In another example, an adult patient may require 1 unit of blood wherein a unit of blood is 1200 mL.

According to some embodiments, the blood is collected directly from a subject into the blood collection and autotransfusion container. According to some embodiments, the blood is collected indirectly from a subject. According to some embodiments, the blood is directly collected from a subcutaneous access site in a subject. According to some embodiments, the blood is collected indirectly from a drainage device that drains blood from a subject. According to some embodiments, blood is collected by gravitational forces or by suction forces. According to some embodiments, blood is forced into the blood collection and autotransfusion container through gravitational forces. According to some embodiments, blood is forced into the blood collection and autotransfusion container through suction forces.

According to some embodiments, blood is collected by regulating the pressure differentials resulting from collecting or draining blood from a subject. According to some embodiments, regulating the pressure differentials resulting from collecting or draining blood from a subject is effective to release the build up of positive pressure in the drainage device or in the container. According to some embodiments, regulating the pressure differentials resulting from collecting or draining blood from a subject is effective to invoke or maintain negative pressure in the body of a subject.

According to some embodiments, blood is transfused by applying pressure or force to the collected blood. According to some embodiments, applying pressure or force to the collected blood is effective to force the blood back into the patient.

According to some embodiments, the blood collection and autotransfusion container comprises a preservative. According to some embodiments, the blood collection and autotransfusion container is effective for short term storage or long-term storage of blood or blood products. For example, studies have shown that whole blood may be stored at controlled room temperature (+22° C.±2° C.) for up to 24 hours after collection without deleterious effects on factor VIII yield, or on the properties of red cells and platelets According to some embodiments, if utilized for short-term storage, the blood collection and autotransfusion container optionally comprising the preservative is effective for preservation of living cells, while reducing undesirable chemical reactions (such as oxidation). According to some embodiments, if utilized for long-term storage, a cryoprotectant (e.g., glycerol, DMSO) may be used to prevent cellular freezing damage when very low temperatures are used to preserve structurally intact living cells and tissues).

According to some embodiments, the blood collection and autotransfusion container comprises an anticoagulant solution. According to some embodiments, the anticoagulant solution comprises CPD, CPDA-1, SAGM, AS-1, AS-3, or AS-5. For example, citrate phosphate dextrose (CPD) a solution containing citric acid, sodium citrate, monobasic sodium phosphate, and dextrose, is the primary anticoagulant used for preservation of whole blood or red blood cells for up to 21 days. The official USP name is anticoagulant citrate phosphate dextrose solution. Citrate phosphate dextrose adenine (CPDA-1), an anticoagulant solution containing citric acid, sodium citrate, monobasic sodium phosphate, dextrose, and adenine, is used for the preservation of whole blood and red blood cells for up to 35 days; it extends red cell survival by providing adenine needed for the maintenance of red cell ATP levels. The official USP name is anticoagulant citrate phosphate dextrose adenine solution. An additive solution containing combinations of saline, adenine, glucose and mannitol (SAGM) provides extended shelf life of RBC up to 42 days with increased functional viability. AS-1 and AS-5 (widely used in the USA) are two SAGM variants which differ only modestly in their concentrations of salt, sugar and mannitol. AS3 also contains citrate and phosphate. Citrate and mannitol serve the same membrane protective function in AS-3 and SAGM, respectively. AS-3 depends on a version of the primary CPD anticoagulant with higher dextrose content called CP2D. (See D'Amici, GM et al., Blood Transfus. (2012) 10 (Suppl. 2): s46-s54.

According to some embodiments, the blood collection and autotransfusion container comprises an amount of the anticoagulant solution sufficient to effect anticoagulation or antithrombotic properties. According to some embodiments, the blood collection and autotransfusion container comprises a 1:1, 1:2, 1:3, 1:4, 1:5, 1:6, 1:7, 1:8, 1:9, 1:10 (v/v), ratio of anticoagulant solution to blood or blood products. According to some embodiments, the blood collection and autotransfusion container comprises a 1:5, 1:6, 1:7, or 1:8 ratio (v/v) of anticoagulant solution to blood or blood products. For example, the blood collection and autotransfusion container comprises about 63 mL of anticoagulant solution for every 450 mL of blood or blood products.

According to some embodiments, the blood collection and autotransfusion container comprises a blood filter wherein the blood filter fluidly communicates with the blood collection and autotransfusion container.

According to some embodiments, the anticoagulant is an anticoagulant coating. According to some embodiments, the inner surface of the blood collection and autotransfusion container is configured to be coated with the anticoagulant coating Exemplary anticoagulant coatings include citrate phosphate-dextrose (CPD), CPD-adenine (CDPA-1), citrate phosphate double dextrose (CP2D); heparin; polymers, such as polyurethanes, polyethylene glycols (PEGs); copolymers such as a poloxamer (meaning an in-block copolymer of poly(ethylene oxide)poly(propylene oxide)-polyethylene oxide sold commercially as Pluronic®), graphene, tricalcium phosphate; positive, negative, and neutral charged coatings; hydromer materials, such as polyvinylpyrrolidone (povidone); nonpolymeric material such as catecholamines (e.g., DA51-cat), hyaluronic acid, polydopamine, peptides (e.g., AMP), combinations thereof see, e.g., Wei, Houliang, et al. “Anticoagulant Surface Coating Using Composite Polysaccharides with Embedded Heparin-Releasing Mesoporous Silica.” ACS Applied Materials & Interfaces, U.S. National Library of Medicine, 11 Dec. 2013, https://www.ncbi.nlm.nih.gov/pubmed/24224889); and Meledeo, Michael Adam, et al. “Optimizing Whole Blood Storage: Hemostatic Function of 35-Day Stored Product in CPD, CP2D, and CPDA-1 Anticoagulants.” Transfusion, U.S. National Library of Medicine, April 2019, https://www.ncbi.nlm.nih.gov/pubmed/30980756).

According to some embodiments, the blood collection and autotransfusion container comprises an antimicrobial agent. Antimicrobial agents are known in the art, see for example, Hadjesfandiari, and Narges. “Improving Platelet Storage Bags: Antifouling Polymer Coatings, Antimicrobial Peptides and Surface Topography.” Open Collections, University of British Columbia, 1 Jan. 1970, https://open.library.ubc.ca/cIRcle/collections/ubctheses/24/items/1.0348313; see also Hadjesfandiari, Narges, et al. “Development of Antifouling and Bactericidal Coatings for Platelet Storage Bags Using Dopamine Chemistry.” Advanced Healthcare Materials, U.S. National Library of Medicine, March 2018, https://www.ncbi.nlm.nih.gov/pubmed/28961393, the entireties of which are incorporated herein by reference.

An exemplary method for confirming sterility of the inner chamber of the blood collection and autotransfusion container comprises flushing the blood collection and autotransfusion container with a sterile fluid; the eluate then is membrane filtered and placed into FTM and SCDM media and incubated to detect microbial growth. Another method for confirming sterility comprises: (a) sterilely collecting a blood or blood product into the blood collection and autotransfusion container; (b) gently and slowly rocking the blood or blood product to allow contact with the inner walls of the blood collection and autotransfusion container; (c) removing an aliquot of the blood or blood product into sterile containers for testing for microbial contamination; and (d) analyzing the aliquot for microbial contamination. According to some embodiments, a sterile penetration member is used to remove the sample or blood or blood products from the blood collection and autotransfusion container via the infusion member, transfusion member, or any suitable component thereof.

According to some embodiments, the blood collection and autotransfusion container is configured for one time use. According to some embodiments, the blood collection and autotransfusion container is configured for more than one use per subject. According to some embodiments, the blood collection and autotransfusion container is configured for 10 uses, 9 uses. 8 uses, 7 uses, 6 uses, 5 uses, 4 uses, 3 uses, 2 uses, 1 use per subject. According to some embodiments, the blood collection and autotransfusion container is configured for more than one use per subject dependent on the capacity of the blood collection and autotransfusion container, the patient population, and the needs of the patient. For example, if a neonatal patient requires 9 units of blood, where one unit of blood is 25 mL, then the blood collection and autotransfusion container is configured for 9 uses. In another example, if a neonatal patient requires 9 units of blood where one unit of blood is 30 mL, then the blood collection and autotransfusion container is configured for 9 uses. In another example, if a neonatal patient requires 9 units of blood where one unit of blood is 35 mL, then the blood collection and autotransfusion container is configured for 9 uses. In another example, an infant patient requires 6 units of blood where one unit of blood is 40 mL, then the blood collection and autotransfusion container is configured for 6 uses. In another example, an infant patient requires 6 units of blood where one unit of blood is 45 mL, then the blood collection and autotransfusion container is configured for 6 uses. In another example, an infant patient requires 6 units of blood where one unit of blood is 50 mL, then the blood collection and autotransfusion container is configured for 6 uses. In another example, if a child patient requires 3 units of blood where one unit of blood is 80 mL, then the blood collection and autotransfusion container is configured for 3 uses. In another example, if a child patient requires 3 units of blood where one unit of blood is 85 mL, then the blood collection and autotransfusion container is configured for 3 uses. In another example, if a child patient requires 3 units of blood where one unit of blood is 90 mL, then the blood collection and autotransfusion container is configured for 3 uses. In another example, if a child patient requires 3 units of blood where one unit of blood is 95 mL, then the blood collection and autotransfusion container is configured for 3 uses. In another example, if a child patient requires 3 units of blood where one unit of blood is 100 mL, then the blood collection and autotransfusion container is configured for 3 uses. In another example, if an adult patient requires 5 units of blood where if a unit of blood is 200 mL, then the blood collection and autotransfusion container is configured for 5 uses. In another example, if an adult patient requires 4 units of blood where a unit of blood is 450 mL, then the blood collection and autotransfusion container is configured for 4 uses. In another example, if an adult patient requires 3 units of blood where a unit of blood is 700 mL, then the blood collection and autotransfusion container is configured for 3 uses. In another example, if an adult patient requires 2 units of blood where a unit of blood is 950 mL, then the blood collection and autotransfusion container is configured for 2 uses. In another example, if an adult patient requires 1 unit of blood where a unit of blood is 1200 mL, then the blood collection and autotransfusion container is configured for 1 use. According to some embodiments, the blood collection and autotransfusion container is disposable.

According to some embodiments, 1 blood collection and autotransfusion container per subject is configured for the collection of blood from a subject and the transfusion of blood or blood products to the subject. According to some embodiment, more than 1 blood collection and autotransfusion container per subject is configured for the collection of blood from a subject and the transfusion of blood or blood products to the subject. According to some embodiment, more than 1, more than 2, more than 3, more than 4, more than 5, more than 6, more than 7, more than 8, more than 9, and the like, blood collection and autotransfusion containers per subject is/are configured for the collection of blood from a subject and the transfusion of blood or blood products to the subject. According to some embodiments, the use of more than 1 blood collection and autotransfusion container per subject is dependent on the capacity of the blood collection and autotransfusion container, the patient population, and the needs of the patient. For example, if a neonatal patient requires 9 units of blood, where one unit of blood is 25 mL, then 9 blood collection and autotransfusion containers are configured for 1 use each; or if a neonatal patient requires 9 units of blood, where one unit of blood is 25 mL, then 3 blood collection and autotransfusion containers are configured for 3 uses each; or if a neonatal patient requires 9 units of blood where one unit of blood is 25 mL, then 2 blood collection and autotransfusion containers are configured where the first blood collection and autotransfusion container is configured for 4 uses and the second blood collection and autotransfusion container is configured for 5 uses; or if a neonatal patient requires 9 units of blood where one unit of blood is 25 mL, then 1 blood collection and autotransfusion container is configured for 9 uses. In another example, if a neonatal patient requires 9 units of blood where one unit of blood is 30 mL, then 9 blood collection and autotransfusion containers are configured for 1 use each; or if a neonatal patient requires 9 units of blood where one unit of blood is 30 mL, then 3 blood collection and autotransfusion containers are configured for 3 uses each; or if a neonatal patient requires 9 units of blood where one unit of blood is 30 mL, then 2 blood collection and autotransfusion containers are configured where the first blood collection and autotransfusion container is configured for 4 uses and the second blood collection and autotransfusion container is configured for 5 uses; or if a neonatal patient requires 9 units of blood where one unit of blood is 30 mL, then 1 blood collection and autotransfusion containers is configured for 9 uses. In another example, if a neonatal patient requires 9 units of blood where one unit of blood is 35 mL, then 9 blood collection and autotransfusion containers are configured for 1 use each; or if a neonatal patient requires 9 units of blood where one unit of blood is 35 mL, then 3 blood collection and autotransfusion containers are configured for 3 uses each; or if a neonatal patient requires 9 units of blood where one unit of blood is 35 mL, then 2 blood collection and autotransfusion containers are configured where the first blood collection and autotransfusion container is configured for 4 uses and the second blood collection and autotransfusion container is configured for 5 uses; or if a neonatal patient requires 9 units of blood where one unit of blood is 35 mL, then 1 blood collection and autotransfusion container is configured for 9 uses. In another example, if an infant patient requires 6 units of blood where one unit of blood is 40 mL, then 6 blood collection and autotransfusion containers are configured for 1 use each; or if an infant patient requires 6 units of blood where one unit of blood is 40 mL, then 3 blood collection and autotransfusion containers are useful for two uses each; or if a neonatal patient requires 6 units of blood where one unit of blood is 40 mL, then 2 blood collection and autotransfusion containers are configured for 3 uses each; or if a neonatal patient requires 6 units of blood where one unit of blood is 40 mL, then 1 blood collection and autotransfusion container is configured for 6 uses. In another example, if an infant patient requires 6 units of blood where one unit of blood is 45 mL, then 6 blood collection and autotransfusion containers are configured for 1 use each; or if an infant patient requires 6 units of blood where one unit of blood is 45 mL, then 3 blood collection and autotransfusion containers are configured for two uses each; or if a neonatal patient requires 6 units of blood where one unit of blood is 45 mL, then 2 blood collection and autotransfusion containers are configured for 3 uses each; or if a neonatal patient requires 6 units of blood where one unit of blood is 45 mL, then 1 blood collection and autotransfusion containers are configured for 6 uses._In another example, an infant patient requires 6 units of blood where one unit of blood is 50 mL, then 3 blood collection and autotransfusion containers are configured for two uses each; or if an infant patient requires 6 units of blood where one unit of blood is 50 mL, then 2 blood collection and autotransfusion containers are configured for three uses each; or if an infant patient requires 6 units of blood where one unit of blood is 50 mL, then 1 blood collection and autotransfusion containers are configured for 6 uses. In another example, if a child patient requires 3 units of blood where one unit of blood is 80 mL, then 3 blood collection and autotransfusion containers are configured for 1 use each; or if a child patient requires 3 units of blood where one unit of blood is 80 mL, then 2 blood collection and autotransfusion containers are configured where the first blood collection and autotransfusion container is configured for 2 uses and the second blood collection and autotransfusion container is configured for 1 use; or if a child patient requires 3 units of blood where one unit of blood is 80 mL, then 1 blood collection and autotransfusion containers is configured for 3 uses. In another example, if a child patient requires 3 units of blood where one unit of blood is 85 mL, then 3 blood collection and autotransfusion containers are useful for 1 use each; or if a child patient requires 3 units of blood where one unit of blood is 85 mL, then 2 blood collection and autotransfusion containers are useful where the first blood collection and autotransfusion container is configured for 2 uses and the second blood collection and autotransfusion container is configured for 1 use; or if a child patient requires 3 units of blood where one unit of blood is 85 mL, then 1 blood collection and autotransfusion container is configured for 3 uses. In another example, if a child patient requires 3 units of blood where one unit of blood is 90 mL, then 3 blood collection and autotransfusion containers are useful for 1 use each; if a child patient requires 3 units of blood where one unit of blood is 95 mL, then 2 blood collection and autotransfusion containers are useful where the first blood collection and autotransfusion container is configured for 2 uses and the second blood collection and autotransfusion container is configured for 1 use; or if a child patient requires 3 units of blood where one unit of blood is 90 mL, then 1 blood collection and autotransfusion container is configured for 3 uses. In another example, if a child patient requires 3 units of blood where one unit of blood is 95 mL, then 3 blood collection and autotransfusion containers are useful for 1 use each; if a child patient requires 3 units of blood where one unit of blood is 95 mL, then 2 blood collection and autotransfusion containers are useful where the first blood collection and autotransfusion container is configured for 2 uses and the second blood collection and autotransfusion container is configured for 1 use; or if a child patient requires 3 units of blood where one unit of blood is 95 mL, then 1 blood collection and autotransfusion container is configured for 3 uses. In another example, if a child patient requires 3 units of blood where one unit of blood is 100 mL, then 3 blood collection and autotransfusion containers are useful for 1 use each; if a child patient requires 3 units of blood where one unit of blood is 100 mL, then 2 blood collection and autotransfusion containers are useful where the first blood collection and autotransfusion container is configured for 2 uses and the second blood collection and autotransfusion container is configured for 1 use; or if a child patient requires 3 units of blood where one unit of blood is 100 mL, then 1 blood collection and autotransfusion container is configured for 3 uses. In another example, if an adult patient requires 5 units of blood where a unit of blood is 200 mL, then 5 blood collection and autotransfusion containers are useful for 1 use each; or if an adult patient requires 5 units of blood where a unit of blood is 200 mL, then 3 blood collection and autotransfusion containers are useful where the first and second blood collection and autotransfusion container is configured for 2 uses, and the third blood collection and autotransfusion container is configured for 1 use; or if an adult patient requires 5 units of blood where a unit of blood is 200 mL, then 2 blood collection and autotransfusion containers are useful where the first blood collection and autotransfusion container is configured for 2 uses and the second blood collection and autotransfusion container is configured for 3 uses; or if an adult patient requires 5 units of blood where a unit of blood is 200 mL, or if an adult patient requires 5 units of blood where a unit of blood is 200 mL, then 1 blood collection and autotransfusion container is configured for 5 uses. In another example, if an adult patient requires 4 units of blood where a unit of blood is 450 mL, then 4 blood collection and autotransfusion containers are useful for 1 use each; or if an adult patient requires 4 units of blood where a unit of blood is 450 mL, then 2 blood collection and autotransfusion containers are useful for 2 uses each; or if an adult patient requires 4 units of blood where a unit of blood is 450 mL, then 1 blood collection and autotransfusion container is configured for 4 uses. In another example, if an adult patient requires 3 units of blood where a unit of blood is 700 mL, then 3 blood collection and autotransfusion containers are useful for 1 use each; if an adult patient requires 3 units of blood where a unit of blood is 700 mL, then 2 blood collection and autotransfusion containers where the first blood collection and autotransfusion container is configured for 2 uses and the second blood collection and autotransfusion container is configured for 1 use; or if an adult patient requires 3 units of blood where a unit of blood is 700 mL, then 1 blood collection and autotransfusion container is configured for 3 uses. In another example, if an adult patient requires 2 units of blood where a unit of blood is 950 mL, then 2 blood collection and autotransfusion containers are useful are useful for 2 uses each; or if an adult patient requires 2 units of blood where a unit of blood is 950 mL, then 1 blood collection and autotransfusion container is useful 2 uses. In another example, if an adult patient requires 1 unit of blood where a unit of blood is 1200 mL, then 1 blood collection and autotransfusion container is configured for 1 use.

According to some embodiment, if more than 1 blood collection and autotransfusion container is used for the collection of blood from a subject and the transfusion of blood or blood products to the subject, then the more than 1 blood collection and autotransfusion containers may be used concurrently and alternatively to effect the collection of blood from a subject and the transfusion of blood or blood products to the subject. For example, if 2 blood collection and autotransfusion containers are used then a first blood collection and autotransfusion container is used for the collection of blood from a subject and the transfusion of blood or blood products to the subject, and a second blood collection and autotransfusion container is used for the collection of blood from a subject while the first blood collection and autotransfusion container is transfusing the collected blood back to the subject; once the first blood collection and autotransfusion container is emptied and/or the second blood collection and autotransfusion container is filled then the second blood collection and autotransfusion container is used for the transfusion of blood or blood products to the subject, and if required, the first blood collection and autotransfusion container is used for the collection of blood from a subject while the second blood collection and autotransfusion container is transfusing the collected blood back to the subject. In another example, if 3 blood collection and autotransfusion containers are used then a first blood collection and autotransfusion container is used for the collection of blood from a subject and the transfusion of blood or blood products to the subject, and a second blood collection and autotransfusion container is used for the collection of blood from a subject while the first blood collection and autotransfusion container is transfusing the collected blood back to the subject, and a third blood collection and autotransfusion container is waiting on standby to be used; once the first blood collection and autotransfusion container is emptied and/or the second blood collection and autotransfusion container is filled then the second blood collection and autotransfusion container is used for the transfusion of blood or blood products to the subject, and the third blood collection and autotransfusion container is used for the collection of blood from a subject while the second blood collection and autotransfusion container is transfusing the collected blood back to the subject, and optionally the first blood collection and autotransfusion container is waiting on standby to be used. In another example, if 4 blood collection and autotransfusion containers are used then a first and second blood collection and autotransfusion containers are used for the collection of blood from a subject and the transfusion of blood or blood products to the subject, and a third and fourth blood collection and autotransfusion container is used for the collection of blood from a subject while the first blood collection and autotransfusion container is transfusing the collected blood back to the subject; once the first and second blood collection and autotransfusion containers are emptied and/or the third and fourth blood collection and autotransfusion containers are filled then the third and fourth blood collection and autotransfusion container is used for the transfusion of blood or blood products to the subject, and if required, the first and second blood collection and autotransfusion containers are used for the collection of blood from a subject while the third and fourth blood collection and autotransfusion containers are transfusing the collected blood back to the subject.

According to some embodiments, the blood collection and autotransfusion container is effective for use in various patient populations, such as, neonatal, infant, child, adults, and/or geriatric patients. According to some embodiments, the blood collection and autotransfusion container of the described invention can be used in a various settings, such as in emergency and/or trauma care at the site of an emergency (a.k.a. in the field); emergency and/or trauma care in a healthcare environment, such as acute-care hospitals; urgent care centers; long-term care facilities, such as nursing homes and nursing facilities, outpatient clinics; and home healthcare. According to some embodiments, the blood collection and autotransfusion container of the described invention can be used in a variety of circumstances, such as, emergency or trauma situations; pre-operative, intra-operative, and post-operative, intensive-care situations; and etc. According to some embodiments, the blood collection and autotransfusion container of the described invention can be used by a variety of healthcare providers, such as first responders, paramedics, medics, armed forces medics, doctors of medicine or osteopathy, nurses, nurse-practitioners, midwives, home healthcare providers, and the like.

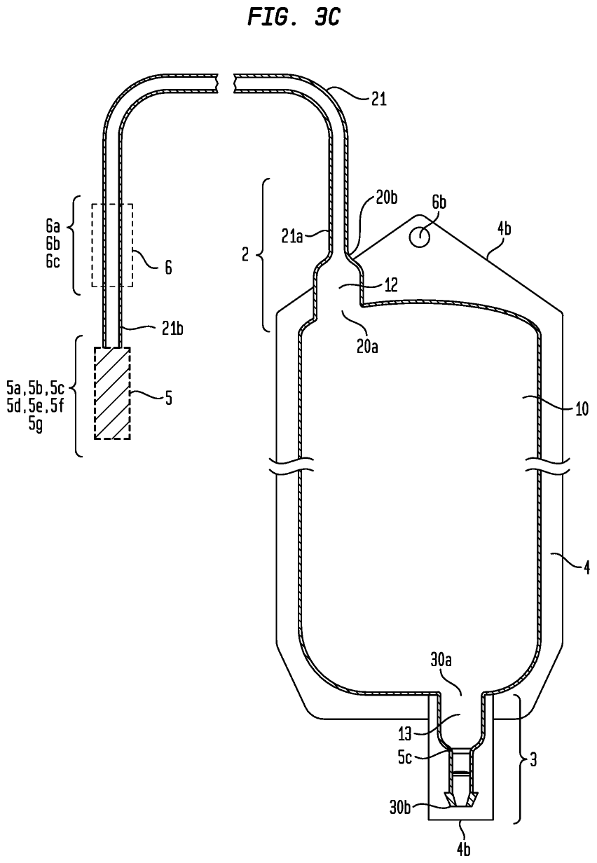

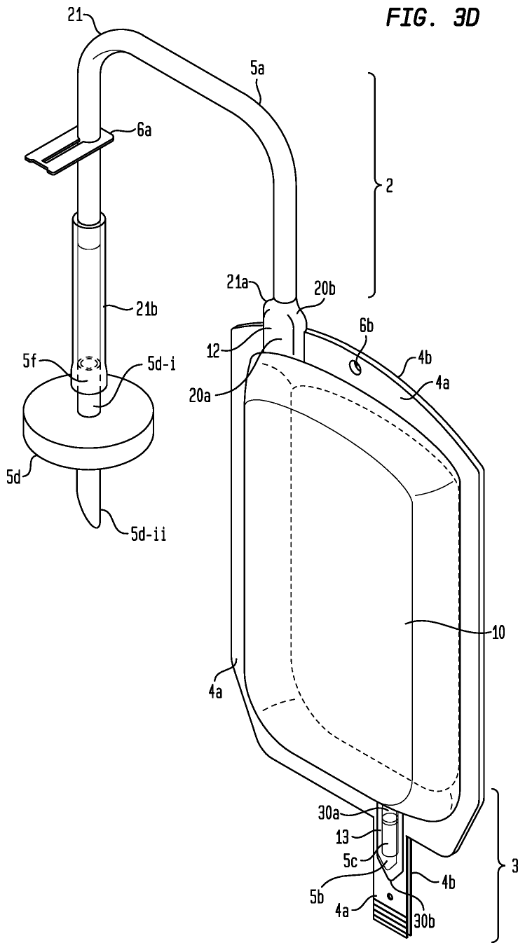

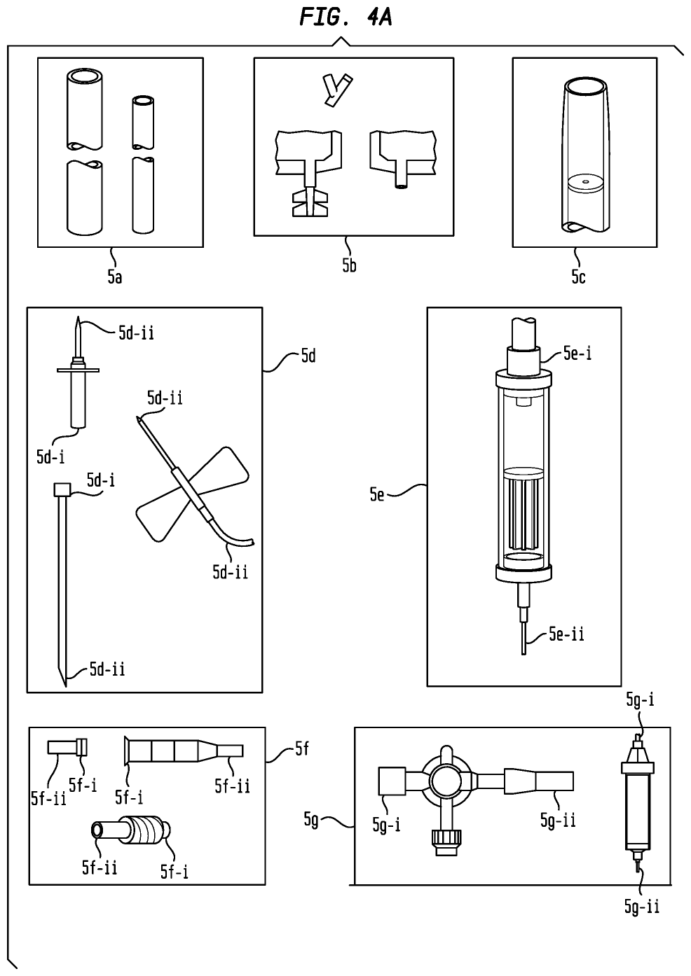

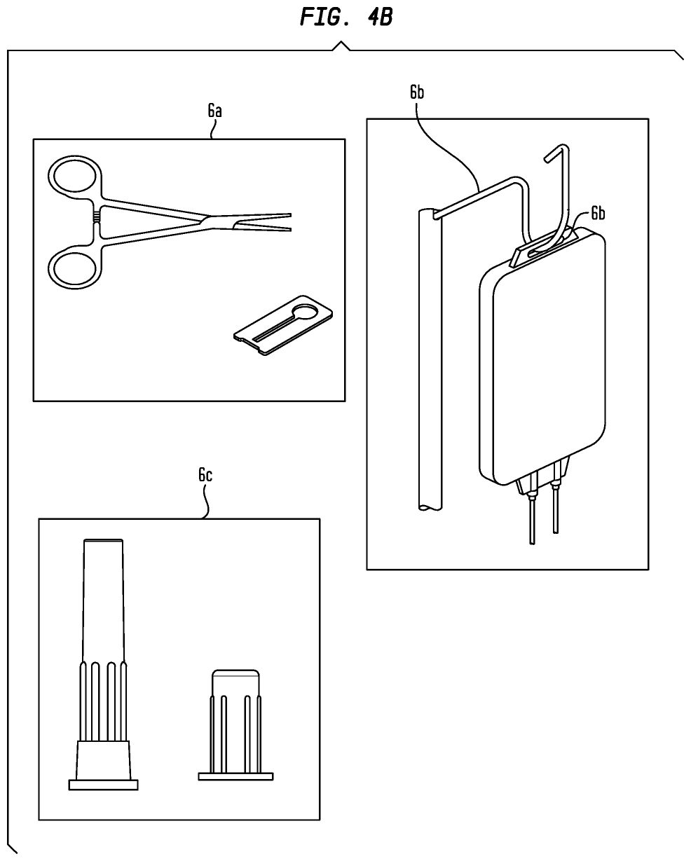

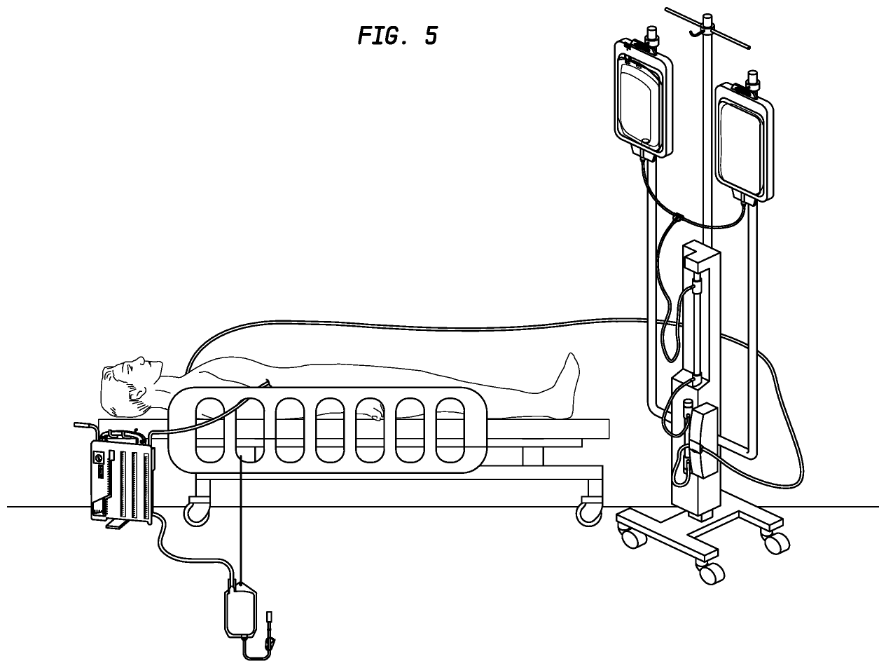

According to some embodiments, the described invention relates to the blood collection and autotransfusion container shown in FIGS. 3A, 3B, 3C, 3D and 4.

According to some embodiments, the blood collection and autotransfusion container comprises a blood collection and autotransfusion container 1 comprising;
i. at least one infusion member 2; and
ii. at least one transfusion member 3.

According to some embodiments, the at least one infusion member 2 is in fluid communication with the blood container 1. According to some embodiments, the at least one transfusion member 3 is in fluid communication with blood container 1.

According to some embodiments, the blood collection and autotransfusion container is sealed so that it further comprises edges 4 comprising of a front edge 4a and a back edge, 4b wherein front edge 4a and back edge 4b are separably or non-separably contiguous to create the seal. According to some embodiments, front edge 4a is non-separably contiguous to back seal edge 4b. According to some embodiments, front edge 4a is separably contiguous to back seal edge 4b. According to these embodiments, front edge 4a and back edge 4b can be grasped and pulled apart or cut in order to unseal a portion of the blood collection and autotransfusion container. According to some embodiments, front edge 4a comprises a front tab 4a-i, and back edge 4b comprises a back tab 4b-i. According to some embodiments, front tab 4a-i and back tab 4b-i are configured to interdigitate.

According to some embodiments, blood collection and autotransfusion container further comprises at least one tubing fitting 5. According to some embodiments, the at least one tubing fitting 5 is used to describe any component or object that is connected to the blood collection and autotransfusion container described herein or any component thereof, wherein the at least one tubing fitting 5 is configured to allow for the potential of fluid communication between two or more objects or components. According to some embodiments, the at least one tubing fitting 5 is configured to allow fluid communication between two or more objects or components. According to some embodiments, the at least one tubing fitting 5 is configured to fully or partially occlude fluid communication between two or more objects or components.

According to some embodiments, at least one infusion member 2 comprises at least one tubing fitting 5. According to some embodiments, at least one transfusion member 3 comprises at least one tubing fitting 5.

As seen in FIGS. 3A, 3B, 3C, 3D, and 4, according to one embodiment, tubing fitting 5 comprises a tube member 5a that is configured to be in fluid communication with the blood collection and autotransfusion container. According to some embodiments, tube member 5a is configured to be penetrable to allow for the flow of blood to and from the blood collection and autotransfusion container. According to some embodiments, tube member 5a can be penetrated to allow for the inflow or outflow of fluids. According to some embodiments, tube member 5a is in fluid communication with blood container 1. According to some embodiments, tube member 5a is in fluid communication with at least one infusion member 2. According to some embodiments, tube member 5a is in fluid communication with at least one infusion member 2 via the infusion passageway 12. According to some embodiments, tube member 5a is connected to at least one infusion member 2. According to some embodiments, tube member 5a is in fluid communication with at least one transfusion member 3. According to some embodiments, tube member 5a is in fluid communication with at least transfusion member 3 via the transfusion passageway 13. According to some embodiments, tube member 5a is connected to at least one transfusion member 3.

The tube member 5a can be formed as an elongated structure in a cylindrical shape with a hollow interior, by molding, casting, machining, or by any known method to create the desired structure. The tube member 5a can be formed during the manufacturing process of the blood collection and autotransfusion container or formed as a separate component and later affixed to the blood collection and autotransfusion container by methods such as bonding, adhesives, or machining.

The tube member 5a can be formed from any materials that provide additional particular features, such as having high biocompatibility; having low thrombogenicity; high resistance to light, radiation, low temperatures, high temperatures, moisture, abrasion, cracking, kinks, leaks, chemicals, fungal growth, bacterial growth; exhibits flexibility, tensile strength, or the like, or any combination of the above. For example, tube member 5a can be formed from polyvinyl chlorides (PVC), polyurethanes, thermoplastic poluerathanes (TPUs), polyvinylidene fluoride (PVF), polyethylene, ethylene vinyl acetates (EVAs) silicone, rubber, polypropelenes, fluorinated ethylene propylene (FEP), synthetics thereof (such as Teflon™, Kynar®, Silcryn™), and the like.

The tube member 5a can be formed from any materials that provide particular physical characteristics, such as being transparent, clear, semi-transparent, transparent colored, natural colored, amber colored, opaque colored, and multi-colored.

The tube member 5a can be configured to provide particular features, such as having any suitable length, thickness, inner diameter, and outer diameter to effect biocompatibility; and to effect rapid fluid communication, such as: the rapid infusion of blood within 8 minutes, within 7 minutes, within 6 minutes, within 5 minutes, within 4 minutes, within 3 minutes, within 2 minutes, within 1 minute; and/or the rapid transfusion of blood within 6 minutes, within 5 minutes, within 4 minutes, within 3 minutes, within 2 minutes, within 1 minute. Tube member 5a may be any length for example, the values listed in Table 7 appending the present application. Tube member 5a may be any suitable inner diameter, outer diameter and wall thickness. Exemplary dimensions are listed in Table 8a, Table 8b, Table 8c, and Table 8d appending the present application.

As seen in FIGS. 3A, 3B, 3C, 3D, and 4A, according to one embodiment, tubing fitting 5 comprises a port 5b that is configured to channel the flow of blood to and from the blood collection and autotransfusion container. According to some embodiments, port member 5b is configured to be penetrable to allow for the flow of blood to and from the blood collection and autotransfusion container. According to some embodiments, port member 5b can be penetrated to allow for the inflow or outflow of blood. According to some embodiments, port member 5b is positioned inside the blood collection and autotransfusion container. According to some embodiments, port member 5b is positioned inside the at least one infusion member 2. According to some embodiments, port member 5b channels the flow of blood through infusion passageway 12. According to some embodiments, port member 5b is positioned inside the at least one transfusion member 3. According to some embodiments, port member 5b channels the flow of blood through transfusion passageway 13.

The port member 5b can be formed as a truncated structure in a cylindrical shape with a hollow interior, by molding, casting, machining, or by any known method to create the desired structure. The port member 5b can be formed during the manufacturing process of the blood collection and autotransfusion container or formed as a separate component and later affixed to the blood collection and autotransfusion container by methods such as bonding, adhesives, or machining.

The port member 5b can be formed from any materials that provide additional particular features, such as having high biocompatibility; having low thrombogenicity; high resistance to light, radiation, low temperatures, high temperatures, moisture, abrasion, cracking, kinks, leaks, chemicals, fungal growth, bacterial growth; exhibits structure integrity, flexibility, tensile strength, or the like or any combination of the above. For example, port member 5b can be formed from polyvinyl chlorides (PVC), polyurethanes, thermoplastic poluerathanes (TPUs), polyvinylidene fluoride (PVF), polyethylene, ethylene vinyl acetates (EVAs) silicone, rubber, polypropelenes, fluorinated ethylene propylene (FEP), synthetics thereof (such as Teflon™, Kynar®, Silcryn™), and the like.

The port member 5b can be formed from any materials that provide particular physical characteristics, such as being transparent, clear, semi-transparent, transparent colored, natural colored, amber colored, opaque colored, and multi-colored.

The port member 5b can be configured to provide particular features, such as having any suitable length, thickness, inner diameter, and outer diameter to effect biocompatibility; and to effect rapid fluid communication, such as: the rapid infusion of blood within 8 minutes, within 7 minutes, within 6 minutes, within 5 minutes, within 4 minutes, within 3 minutes, within 2 minutes, within 1 minute; and/or the rapid transfusion of blood within 6 minutes, within 5 minutes, within 4 minutes, within 3 minutes, within 2 minutes, within 1 minute. According to some embodiments, suitable dimensions for port member 5b are dimensions such that port member 5b can connect to one or more components and/or objects and still tallow for fluid communication. For example, if port member 5b connects to tube member 5a, then port member 5b must have an external diameter configured to connect to tube member 5a, and an internal diameter configured to allow fluid communication between port member 5b and tube member 5a. Exemplary inner diameter for port member 5b may be found in Table 9 appending the present application.

As seen in FIGS. 3A, 3B, 3C, 3D, and 4A, according to one embodiment, tubing fitting 5 comprises a membrane member 5c that is configured to occlude the flow of blood from the blood collection and autotransfusion container. According to some embodiments, membrane member 5c is configured to be penetrable to allow for the flow of blood to and from the blood collection and autotransfusion container. According to some embodiments, membrane member 5c is configured to prevent contamination of the blood collection and autotransfusion container by limiting access to the interior of the blood collection and autotransfusion container 11. According to some embodiments, membrane member 5c can be penetrated to allow for the inflow or outflow of fluids. According to some embodiments, membrane member 5c can self-seal after removal of the penetrating object thereof. According to some embodiments, membrane member 5c is positioned inside the blood collection and autotransfusion container. According to some embodiments, membrane member 5c is positioned inside the at least one infusion member 2. According to some embodiments, membrane 5c is positioned inside the at least one transfusion member 3. According to some embodiments, membrane 5c occludes the flow of blood through transfusion passageway 13.

According to some embodiments, the membrane member 5c can be formed as a circular structure in a disc-like shape with a solid interior, by molding, casting, machining, or by any known method to create the desired structure. According to some embodiments, the membrane member 5c can be formed during the manufacturing process of the blood collection and autotransfusion container, or can be formed as a separate component and later affixed to the blood collection and autotransfusion container by methods such as bonding, adhesives, or machining.

The membrane member 5c can be formed from any materials that provide additional particular features, such as having high biocompatibility; having low thrombogenicity; high resistance to light, radiation, low temperatures, high temperatures, moisture, abrasion, cracking, kinks, leaks, chemicals, fungal growth, bacterial growth; exhibits structure integrity, flexibility, tensile strength, or the like or any combination of the above. For example, membrane member 5c can be formed from polyvinyl chlorides (PVC), polyurethanes, thermoplastic poluerathanes (TPUs), polyvinylidene fluoride (PVF), polyethylene, ethylene vinyl acetates (EVAs) silicone, rubber, polypropelenes, fluorinated ethylene propylene (FEP), synthetics thereof (such as Teflon™, Kynar®, Silcryn™), and the like.

The membrane member 5c can be formed from any materials that provide particular physical characteristics, such as being transparent, clear, semi-transparent, transparent colored, natural colored, amber colored, opaque colored, and multi-colored.

The membrane member 5c can be configured to provide particular features, such as having any suitable thickness and outer diameter to effect the occlusion and non-occlusion of fluid communication. According to some embodiments, the membrane member may comprise an outer diameter that is configured to be in sealable contact with the inner diameter of tubing member 5a or port member 5b.

As seen in FIGS. 3A, 3B, 3C, 3D, and 4A, according to one embodiment, tubing fitting 5 comprises a penetrating member 5d that is configured to be connected to a first component or object and penetrate a second component, object or subject and allow for fluid communication between the first component/object and second component/object/subject. According to some embodiments, penetrating member 5d is connected in an airtight manner. According to some embodiments, penetrating member 5d penetrates in an airtight manner.

According to some embodiments, penetrating member 5d is cylindrical shaped with a hollow interior. According to some embodiments, a penetrating member has a first end 5d-i and a second end 5d-ii. According to some embodiments, the first end of penetrating member 5d-i is configured to connect to a first object or a component by fitting into or fitting over the first object or component. According to some embodiments, the first end of penetrating member 5d-i is configured to fit into a first object or a component wherein exterior dimensions of the first end of penetrating member 5d-i are configured to be contiguous with the interior dimensions of the first object or component. According to some embodiments, the second end of penetrating member 5d-ii is configured to penetrate into a second object, component or subject. According to some embodiments, penetrating member 5d tapers from the first end 5d-i to the second end 5d-ii.

According to some embodiments, penetrating member 5d is positioned on the blood collection and autotransfusion container so that fluid flows from a first object or component into penetrating member first end 5d-i, travels through penetrating member 5d, and flows out of penetrating member second end 5d-ii into a second object, component, or subject.

According to some embodiments, the first end of penetrating member 5d-i is a hub. According to some embodiments, the first end of penetrating member 5d-i connects to a hub. According to some embodiments, the second end of penetrating member 5d-ii is a catheter, needle, cannula, medical spike, or a drainage tube.

The penetrating member 5d can be formed as an elongated structure in a cylindrical shape with a hollow interior, by molding, casting, machining, or by any known method to create the desired structure. The penetrating member 5d can be formed during the manufacturing process of the blood collection and autotransfusion container or formed as a separate component and later affixed to the blood collection and autotransfusion container.

The penetrating member 5d can be formed from any materials that provide particular physical characteristics, such as being formed from any biocompatible and anti-microbial plastics and metal alloys. For example, stainless steel.

The penetrating member 5d can be configured to provide particular features, such as being of a suitable gauge, interior diameter, exterior diameter and wall thickness. According to some embodiments, suitable dimensions for penetrating member 5d are dimensions such that penetrating member 5d can penetrate into one or more components and/or objects and still allow fluid communication. For example, if penetrating member 5d penetrates tube member 5a, then penetrating member 5d must have an external diameter configured to penetrate into tube member 5a, an internal diameter configured to allow fluid communication between penetrating member 5d and tube member 5a, and a gauge configured to allow the rapid flow of communication between penetrating member 5d and tube member 5a. Exemplary dimensions are listed in Table 5, and Table 10a and Table 10b appending the present application. For example, the inner diameter of penetrating member 5d is about 0.003 in to about 1.083 in. For example, the inner diameter of penetrating member 5d is about 0.0625 in to about 0.333 in. For example, the gauge of penetrating member 5d is about 3 Fr to about 40 Fr. For example, the gauge of penetrating member 5d is about 8 Fr to about 36 Fr. For example, the gauge of penetrating member is about 14 Fr to about 24 Fr.

As seen in FIG. 4A, according to one embodiment, tubing fitting 5 comprises a filter member 5e that is configured to trap contaminants, foreign materials, and clumps/clots of biological material in the fluid being fluidly communicated. According to one embodiment, filter member 5e is configured to filter fluid, such as, blood, being fluidly communicated. According to one embodiment, filter member 5e is configured to connect to a first object or component and to a second object or component.

According to some embodiments, filter member 5e has a first end 5e-i and a second end 5e-ii. According to some embodiments, the first end of filter member 5e-i is configured to connect to a first object or a component by fitting into or fitting over the first object or component. According to some embodiments, the first end of filter member 5e-i is configured to fit into a first object or a component wherein exterior dimensions of the first end of filter member 5e-i are configured to be contiguous with the interior dimensions of the first object or component. According to some embodiments, the first end of filter member 5e-i is configured to fit over a first object or a component wherein interior dimensions of the first end of filter member 5e-i is configured to be contiguous with the exterior dimensions of the first object or component. According to some embodiments, the second end of filter member 5e-ii is configured to connect to a second object or a component by fitting into or fitting over the second object or component. According to some embodiments, the second end of filter member 5e-ii is configured to fit into a second object or a component wherein exterior dimensions of the second end of filter member 5e-ii is configured to be contiguous with the interior dimensions of the second object or component. According to some embodiments, the second end of filter member 5e-ii is configured to fit over a second object or a component wherein interior dimensions of the second end of filter member 5e-ii are configured to be contiguous with the exterior dimensions of the second object or component.

According to some embodiments, filter member 5e comprises a cavity that has a means to filter fluid, for example through the use of a mesh, wherein the mesh comprises a porous material that traps contaminants, foreign materials, and clumps/clots of biological material but is still efficient to establish fluid communication. Pore size can be any pore size efficient to filter blood, for example, to remove visible particles, white blood cells, mold spores, and bacteria. Pore size is described as the amount of pores in one square inch of the filtering material, and micron size is the pore opening in micrometers. Exemplary pore sizes can be seen in Table 11 appending the application. For example, filter member is 5e about 170-260 microns.

According to some embodiments, filter member 5e is positioned in the blood collection and autotransfusion container so that fluid flows from a first object or component into filter member first end 5e-i, travels through the filter house in filter member 5e, and flows out of filter member second end 5e-ii into a second object or component. According to some embodiments, the first end of filter member 5e-i connects to a tube member 5a. According to some embodiments, the second end of filter member 5e-ii connects to a tube member 5a.

As seen in FIG. 4A, according to one embodiment, tubing fitting 5 is a connector or coupling member 5f that is configured to connect a first component or object with a second component or object, and allow for fluid communication between the first component/object and second component/object. According to some embodiments, connector member 5f connects in an airtight manner. According to some embodiments, connector member is a sleeve, luer, and the like.

According to some embodiments, connector member has a first end 5f-i and a second end 5f-ii. According to some embodiments, the first end of connector member 5f-i is configured to connect to a first object or a component by fitting into or fitting over the first object or component. According to some embodiments, the first end of connector member 5f-i is configured to fit into a first object or a component wherein exterior dimensions of the first end of connector member 5f-i are configured to be contiguous with the interior dimensions of the first object or component. According to some embodiments, the first end of connector member 5f-i is configured to fit over a first object or a component wherein interior dimensions of the first end of connector member 5f-i is configured to be contiguous with the exterior dimensions of the first object or component. According to some embodiments, the second end of connector member 5f-ii is configured connect to a second object or a component by fitting into or fitting over the second object or component. According to some embodiments, the second end of connector member 5f-ii is configured to fit into a second object or a component wherein exterior dimensions of the second end of connector member 5f-ii is configured to be contiguous with the interior dimensions of the second object or component. According to some embodiments, the second end of connector member 5f-ii is configured to fit over a second object or a component wherein interior dimensions of the second end of connector member 5f-ii are configured to be contiguous with the exterior dimensions of the second object or component.

As seen in FIG. 4A, according to one embodiment, tubing fitting 5 comprises a valve member 5g that is configured to connect a first component or object with a second component or object and allow fluid communication at an adjustably slower rate between the first component/object and second component/object than if the first component/object and second component/object were connected without valve member 5g. According to some embodiments, valve member 5g connects in an airtight manner. According to some embodiments, connector member is a valve, or a drip chamber.

According to some embodiments, valve member 5g has a first end 5g-i and a second end 5g-ii. According to some embodiments, the first end of valve member 5g-i is configured connect to a first object or a component by fitting into or fitting over the first object or component. According to some embodiments, the first end of valve member 5g-i is configured to fit into a first object or a component wherein exterior dimensions of the first end of valve member 5g-i are configured to be contiguous with the interior dimensions of the first object or component. According to some embodiments, the first end of valve member 5g-i is configured to fit over a first object or a component wherein interior dimensions of the first end of valve member 5g-i is configured to be contiguous with the exterior dimensions of the first object or component. According to some embodiments, the second end of valve member 5g-ii is configured connect to a second object or a component by fitting into or fitting over the second object or component. According to some embodiments, the second end of valve member 5g-ii is configured to fit into a second object or a component wherein exterior dimensions of the second end of valve member 5g-ii is configured to be contiguous with the interior dimensions of the second object or component. According to some embodiments, the second end of valve member 5g-ii is configured to fit over a second object or a component wherein interior dimensions of the second end of valve member 5g-ii are configured to be contiguous with the exterior dimensions of the second object or component.

According to some embodiments, the blood collection and autotransfusion container comprises one tubing fitting. According to some embodiments, the blood collection and autotransfusion container comprises at least two tubing fittings. According to some embodiments, the blood collection and autotransfusion container comprises at least three tubing fittings. According to some embodiments, the blood collection and autotransfusion container comprises at least four tubing fittings. According to some embodiments, the blood collection and autotransfusion container comprises at least five tubing fittings. According to some embodiments, the blood collection and autotransfusion container comprises at least six tubing fittings. According to some embodiments, a first tubing fitting may contact a second tubing fitting. For example, if the blood collection and autotransfusion container comprises a tube member 5a and a penetrating member 5d, then tube member 5a may contact a penetrating member 5d. According to one aspect, if the blood collection and autotransfusion container comprises a tube member 5a and a penetrating member 5d, wherein penetrating member 5d comprises a first end 5d-i and a second end 5d-ii, then tube member 5a and penetrating member 5d is configured so that the outer diameter of penetrating member 5d contacts with the inner diameter of tube member 5a.

According to some embodiments, blood collection and autotransfusion container further comprises at least one tubing accessory 6. According to some embodiments, the at least one tubing accessory 6 is attached to the blood collection and autotransfusion container or any component thereof and configured to attach onto a portion of the exterior surface of the blood container 1, infusion member 2, and/or transfusion member 3 as described herein or any component thereof without contacting the blood being fluidly communicated therein.

According to some embodiments, blood container 1 comprises at least one tubing accessory 6. According to some embodiments, at least one infusion member 2 comprises at least one tubing accessory 6. According to some embodiments, at least one transfusion member 3 comprises at least one tubing accessory 6.

As seen in FIGS. 3A, 3B, 3C, 3D, and 4B, according to one embodiment, tubing accessory 6 is a clamp member 6a that is configured to adjust fluid communication in one or more objects or components. According to some embodiments, clamp member 6a is configured to allow fluid communication between two or more objects or components. According to some embodiments, clamp member 6a is configured to fully or partially occlude fluid communication between two or more objects or components. According to some embodiments, clamp member 6a is configured to attach to the blood collection and autotransfusion container described herein or any component thereof. According to some embodiments, clamp member 6a is configured to attach to at least one infusion member 2. According to some embodiments, clamp member 6a is configured to attach to at least one transfusion member 3. According to some embodiments, clamp member 6a is configured to attach to at least one tubing fitting 6. For example, clamp member 6a can be configured to attach to tube member 5a.

According to some embodiments, clamp member 6a is configured to externally narrow one or more areas of the object or component to which it is attached, wherein the narrowing action is efficient to allow, occlude or partially occlude fluid communication in and to one or more objects or components and between two or more objects or components. According to some embodiments, clamp member 6a is a pinch clamp, a screw clamp, clamp forceps, roller clamps, slide clamps, flange clamps, and the like.

As seen in FIGS. 3A, 3B, 3C, 3D, and 4B, according to one embodiment, tubing accessory 6 is a hanging member 6b that is configured to hook onto something or be hooked by something in order to be suspended so that it is supported only by the hanging member 6b. According to some embodiments, hanging member 6b is formed through the seal 4. According to some embodiments, hanging member 6b is positioned on the top of blood container 1. According to some embodiments, hanging member 6b is positioned on the left side of blood container 1. According to some embodiments, hanging member 6b is positioned on the right side of blood container 1. According to some embodiments, hanging member 6b is formed through seal 4 on top of blood container 1. According to some embodiments, blood container 1 comprises more than one hanging member 6b. According to some embodiments, blood container 1 comprises two, three, four, five, six, seven, eight, nine, or ten hanging members. According to some embodiments, hanging member 6b is an opening formed through seal 4 on the top edge of blood container 1 (as seen in FIGS. 3A, 3B, 3C, and 3D). According to some embodiments, a hanging member 9 is a hook affixed to seal 4 on top of blood container 1.

As seen in FIG. 4B, according to one embodiment, tubing accessory 6 is a cap member 6c configured to cover a sterile or sterilizable component of the blood collection and autotransfusion container. According to some embodiments, cap member 6c is efficient to maintain the sterility of the covered component.

### Blood Container

According to some embodiments, blood container 1 can be any pliable, flexible, deformable, and/or conformable receptacle appropriate to collect, contain, and transfuse blood or blood products.

A blood container 1 can be formed by any technique suitable, including without limitation, machining, molding, casting, and bonding, and can be formed as a unitary piece or as component pieces attached together by attachment means, such as adhesives, bonding, and machining.

Blood container 1 as a whole or any component thereof can be provided with particular features such as: being formed with any suitable material or with additional components; being formed in any suitable shape, length and width; and being formed to contain any suitable volume.

According to some embodiments, blood container 1 can be formed from any suitable material that is pliable, flexible, deformable, compressible, and/or conformable in two or three dimensional configuration to allow for volume fluctuations.

According to some embodiments, blood container 1 can be formed from any suitable material that is biologically inert; has high resistance to light, radiation, low temperatures, high temperatures moisture, abrasion, cracking, kinks, leaks, chemicals, fungal growth, bacterial growth; exhibits flexibility, pliability, deformability, tensile strength, or etc, or any combination of the above.

According to some embodiments, blood container 1 can be formed from any biocompatible material appropriately inert to biologically active fluids, such as blood, and intravenous solutions. For example, blood container 1 can be formed from nylons, polyesters, fluoroplastics, polyvinyl chlorides, polyethylene, polypropylene, polyimides, polyamides, and the like. According to some embodiments, blood container 1 can be formed from one layer of the above described materials. According to some embodiments, blood container 1 can be formed from more than one layer of the above described materials.

Blood container 1 as a whole or any component thereof can be formed with any suitable volume, depth, length, and width to effect the collection, containment, and transfusion of blood or blood products.

According to some embodiments, blood container 1 can be any volume appropriate to collect, contain, and transfuse blood. According to some embodiments, the volume of blood container 1 can be 20 mL, 25 mL, 35 mL, 50 mL, 75 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 525 mL, 550 mL, 575 mL, 600 mL, 625 mL, 650 mL, 675 mL, 700 mL, 725 mL, 750 mL, 775 mL, 800 mL, 825 mL, 850 mL, 875 mL, 900 mL, 925 mL, 950 mL, 975 mL, 1 L, 1.5 L, and 2 L, inclusive and any value in-between. For example, the volume of blood container 1 can be from about 25 mL to about 1.5 mL.

According to some embodiments, blood container 1 can be any depth appropriate to collect and contain blood based on its volume capacity.

According to some embodiments, blood container 1 can be any length appropriate to collect, contain, and transfuse blood. According to some embodiments, blood container 1 can be any length appropriate for use in an autotransfusion device. According to some embodiments, blood container 1 can be about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, inclusive, and any value in between. For example, blood container 1 can be any length between about 9 cm and about 40 cm.

According to some embodiments, blood container 1 can be any width appropriate to collect, contain, and transfuse blood. According to some embodiments, blood container 1 can be any width appropriate for use in an autotransfusion device. According to some embodiments, blood container 1 can be about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, and about 50 cm, inclusive, and any value in between. For example, blood container 1 can be any width between about 7 cm and about 30 cm.

According to some embodiments, blood container 1 is about 50 to about 250 mL in volume/capacity, about 12.5 cm to about 22.0 cm in length, and about 10.5 cm in width. According to some embodiments, blood container 1 is about 100 mL to about 1000 mL in volume/capacity, about 13.5 cm to about 32.0 cm in length, and about 12.5 cm in width. According to some embodiments, blood container 1 is about 250 mL to about 1200 mL in volume/capacity, about 12.5 cm to about 40.0 cm in length, and about 13.5 cm in width.

According to some embodiments, blood container 1 is in fluid communication with at least one infusion member 2 and with at least one transfusion member 3. According to some embodiments, at least one infusion member 2 is positioned on a first side of blood container 1, and at least one transfusion member 3 is positioned on the first side or on a second side blood container 1. According to some embodiments, at least one infusion member 2 is positioned on the same side or different side on blood container 1 as at least one transfusion member 3. According to some embodiments, the at least one infusion member 2 is positioned on a different side on blood container 1 as the at least one transfusion member 3.

According to some embodiments, blood container 1 is sealed so that it further comprises a front edge 4a and a back edge, 4b. According to some embodiments, blood container 1 is sealed with at least one infusion member 2 and/or with at least one transfusion member 3.

As seen in FIGS. 3A, 3B, 3C, and 3D, according to some embodiments, blood container 1 further comprises a blood collection compartment 10 with a sterile interior, an infusion passageway 12 that communicates with the blood collection compartment interior 10 via the at least one infusion member 2, and a transfusion passageway 13 that communicates with the blood collection compartment interior 10 via the at least one transfusion member 3.

According to some embodiments, the interior of blood collection compartment 10 can be formed or configured to facilitate sterility or hinder the accumulation of contaminants. According to some embodiments, the interior of blood collection compartment 10 is sterile. According to some embodiments, the interior of blood collection compartment 10 can be sterilized. According to some embodiments, the interior of blood collection compartment 10 is airtight. According to some embodiments, the interior of blood collection compartment 10 is coated with an anti-microbial, anti-bacterial, anti-fungal, and anti-septic material. According to some embodiments, the environment-facing exterior surface of blood collection compartment 10 is not sterile.

### Infusion Member

According to some embodiments, infusion member 2 is any structure configured to be appropriate for the inflow of blood from an external source (e.g., from a subject or from a drainage device) into the blood collection compartment 10 via the infusion passageway 12. According to some embodiments, infusion member 2 can be positioned at any location on the blood container 1 to allow and/or facilitate the inflow of blood from an external source. According to one embodiment, infusion member 2 is positioned on the top of blood container 1 and centered. According to one embodiment, infusion member 2 is positioned on the top of blood container 1 on the left side of blood container 1. According to one embodiment, infusion member 2 is positioned on the top of blood container 1 on the right side of blood container 1. According to some embodiments, infusion member 2 comprises at least one tubing fitting 5 and/or at least one tubing accessory 6.

According to some embodiments, infusion member 2 as a whole or any component thereof can be provided with particular features, such as, being formed with any suitable material or with additional components; and such as being formed in any suitable shape, length, width, and height to effect rapid infusion of blood within 9 minutes, within 8 minutes, within 7 minutes, within 6 minutes, within 5 minutes, within 4 minutes, within 3 minutes, within 2 minutes, within 1 minute.

According to some embodiments, infusion member 2 can have any internal diameter to effect the rapid infusion of blood. For example, internal diameter of infusion port 20 is about 3.0 to about 25 mm. For example, internal diameter of infusion member 2 is about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm. For example, internal diameter of infusion member 2 is about 12.00 mm to about 20.00 mm.

As seen in FIGS. 3A, and 3B, according to some embodiments, infusion member 2 comprises an infusion port 20. According to some embodiments, infusion port 20 is port member 5b. According to some embodiments, infusion port 20 encompasses infusion passageway 12. According to some embodiments, infusion port 20 has a first open end 20a and a second open end 20b. According to some embodiments, the first end of infusion port 20a opens into the interior of the blood collection compartment 10, and the second end 20b opens into the exterior of the blood collection compartment. According to some embodiments, the interior of infusion port 20 is sterile. According to some embodiments, the interior of infusion port 20 is contiguous with and extends from the interior of the blood collection compartment 10.

According to some embodiments, infusion port 20 as a whole or any component thereof can be provided with particular features, such as, being formed with any suitable material or with additional components; and such as being formed in any suitable shape, length, width, and height to effect rapid infusion of blood within 8 minutes, within 7 minutes, within 6 minutes, within 5 minutes, within 4 minutes, within 3 minutes, within 2 minutes, within 1 minute.

According to some embodiments, infusion port 20 can have any internal diameter to effect the rapid infusion of blood. Examples of infusion port 20 internal diameters can be seen in Table 9 appending this application. For example, internal diameter of infusion port 20 is about 6.5 to about 25 mm. For example, internal diameter of infusion port 20 is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm. For example, internal diameter of infusion port 20 is about 12.00 mm to about 20.00 mm.

According to some embodiments, infusion port 20 is sealed so that it further comprises edges 4 comprising a front edge 4a and a back edge, 4b wherein front edge 4a and back edge 4b are separably contiguous to create the seal. According to these embodiments, front edge 4a and back edge 4b can be grasped and pulled apart or cut in order to unseal infusion port 20. According to these embodiments, front edge 4a and back edge 4b can be grasped and pulled apart or cut in order to unseal the second end of infusion port 20b. According to some embodiments, front edge 4a comprises a front tab 4a-i, and back edge 4b comprises a back tab 4b-i. According to some embodiments, front tab 4a-i and back tab 4b-i are configured to interdigitate.

According to some embodiments, infusion port 20 further comprises at least one tubing fitting 5 and/or at least one tubing accessory 6. According to some embodiments, tubing fitting 5 is a membrane member 5c. According to some embodiments, membrane member 5c is efficient to occlude the fluid communication in and out of infusion port 20. According to some embodiments, tubing accessory 6 is a cap member 6c. According to some embodiments, cap member 6c is efficient to cover the second end of infusion port 20 to maintain its sterility. According to some embodiments, cap member 6c is efficient to the one or more tubing fittings 5 that may be connected to infusion port 20 to maintain sterility.

According to some embodiments, infusion member 2 comprises an infusion tube 21 that is an elongated structure with a hollow interior that allows for the inflow of blood from an external source through the infusion port 20 into the blood collection compartment 10 of the blood container via the infusion passageway 12. According to some embodiments, infusion tube 21 is tube member 5a. According to some embodiments, infusion tube 21 is sealed so that it further comprises edges 4 comprising of a front edge 4a and a back edge, 4b wherein front edge 4a and back edge 4b are non-separably contiguous to create seal around infusion tube 21. According to some embodiments, infusion tube 21 has a first end 21a and a second end 21b.

According to some embodiments, infusion tube 21 is separably or non-separably connected to infusion port 20b. According to some embodiments, first end of infusion tube 21a is connected to a second end of infusion port 20b. According to some embodiments, first end of infusion tube 21a is non-separably connected to a second end of infusion port 20b. According to some embodiments, the first end of infusion tube 21a is nonseparably connected to the second end of infusion port 20b by being integral with and extending from second end of infusion port 20b.

According to some embodiments, the interior of infusion tube 21 is sterile and therefore maintains the sterility of the interior of blood compartment 10 and the interior of infusion port 20. According to some embodiments, first end of infusion tube 21a is separably connected to a second end of infusion port 20b. According to some embodiments, the first end of infusion tube 21a is separably connected to the second end of infusion port 20b by penetrating into the second end of infusion port 20b.

According to some embodiments, infusion tube 21 further comprises at least one tubing fitting 5 and/or at least one tubing accessory 6. According to some embodiments, infusion tube first end 21a comprises at least one tubing fitting 5 and/or at least one tubing accessory 6. According to some embodiments, infusion tube second end 21b comprises at least one tubing fitting 5 and/or at least one tubing accessory 6. According to some embodiments, tubing fitting 5 is a penetrating member 5d. According to some embodiments, penetrating member 5d is configured to establish fluid communication between infusion tube 21 and a second object or component, such as a subject or a chest drainage device. According to some embodiments, tubing fitting 5 is a filter member 5e. According to some embodiments, filter member 5e is configured to filter fluid that is fluidly communicated between infusion tubing 21 and a second object or component, such as a subject or a chest drainage device. According to some embodiments, tubing fitting 5 is a coupling member 5f. According to some embodiments, coupling member 5f is configured to establish fluid communication between infusion tube 21 and a second object or component, such as a subject or a chest drainage tube. According to some embodiments, tubing accessory 6 is a clamp member 6a. According to some embodiments, clamp member 6a is configured to occlude fluid communication in and out of infusion tube 21. According to some embodiments, tubing accessory 6 is a cap member 6c. According to some embodiments, cap member 6c is configured to cover the second end of infusion tube 21 to maintain its sterility. According to some embodiments, cap member 6c is configured to cover any one or more tubing fittings 5 that may be connected to the second end of infusion tube 21 to maintain sterility.

According to some embodiments, infusion tube 21 as a whole or any component thereof can be provided with particular features, such as, being formed with any suitable material or with additional components; and such as being formed in any suitable shape, length, and width to effect rapid infusion of blood within 8 minutes, within 7 minutes, within 6 minutes, within 5 minutes, within 4 minutes, within 3 minutes, within 2 minutes, within 1 minute.

According to some embodiments, infusion tube 21 may be any length appropriate to effect the rapid infusion of blood. Exemplary lengths can be seen in Table 7 appending this application. For example, infusion tube 21 is about 175 mm, about 200 mm to about 450 mm in length. For example, infusion tube 21 is about 200 mm, about 225 mm, about 250 mm, about 275 mm, about 300 mm, about 325 mm, about 350 mm, about 375 mm, about 400 mm, about 425 mm, or about 450 mm in length. For example, infusion tube 21 is about 200 mm to about 300 mm in length.

According to some embodiments, infusion tube 21 can be configured with any inner diameter, outer diameter, and wall thickness sufficient to accommodate the rapid collection of blood. Exemplary dimensions can be seen in Table 8a, Table 8b, Table 8c, and Table 8d appending this application. For example, the diameter of infusion tube 21 is about 6.5 to about 25 mm, inclusive. For example, the internal diameter of infusion tube 21 is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm in internal diameter. For example, internal diameter of infusion tube 21 is about 12.00 mm to about 20.00 mm.

According to some embodiments, infusion tube 21 can be formed from any material that has high resistance to light, radiation, low temperatures, high temperatures moisture, pressure, abrasion, cracking, kinks, leaks, chemicals, fungal growth, bacterial growth; exhibits flexibly, tensile strength, or etc or any combination of the above. For example, infusion tube 21 can be formed from polyvinyl chlorides (PVC), polyurethanes, thermoplastic poluerathanes (TPUs), polyvinylidene fluoride (PVF), polyethylene, ethylene vinyl acetates (EVAs) silicone, rubber, polypropelenes, fluorinated ethylene propylene (FEP), synthetics thereof (such as Teflon™, Kynar®, Silcryn™).

According to some embodiments, the blood collection and autotransfusion container comprises more than one infusion member 2. According to some embodiments, the blood collection and autotransfusion container comprises at least two infusion members. According to some embodiments, the blood collection and autotransfusion container comprises at least three infusion members. According to some embodiments, the blood collection and autotransfusion container comprises at least four infusion members. According to some embodiments, the blood collection and autotransfusion container comprises at least five infusion members.

### Transfusion Member

According to some embodiments, transfusion member 3 is any structure configured to be appropriate for the rapid outflow of blood from the blood collection compartment 10 of the blood collection and autotransfusion container 1 via the transfusion passageway 13. According to some embodiments, transfusion member 3 can be positioned on any location on the blood container 1 to allow and/or facilitate the outflow of blood. According to one embodiment, if the infusion member is positioned at top center, the transfusion member 3 is positioned at the bottom-center of blood container 1. According to one embodiment, if the infusion member is positioned at top right, the transfusion member 3 is positioned on the bottom-left of blood container 1. According to one embodiment, if the infusion member is positioned at top left, the transfusion member 3 is positioned on bottom-right of blood container 1. According to some embodiments, transfusion member 3 comprises at least one tubing fitting 5 and/or at least one tubing accessory 6.

According to some embodiments, transfusion member 3 as a whole or any component thereof can be provided with particular features, such as, being formed with any suitable material or with additional components; and such as being formed in any suitable shape, length, width, and height to effect the rapid transfusion of blood or blood products within 8 minutes, within 7 minutes, within 6 minutes, within 5 minutes, within 4 minutes, within 3 minutes, within 2 minutes, within 1 minute.

According to some embodiments, transfusion member 3 can have any internal diameter to effect the rapid infusion of blood. For example, internal diameter of transfusion member 3 is about 3.0 to about 25 mm. For example, internal diameter of transfusion member 3 is about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm. For example, internal diameter of transfusion member 3 is about 12.00 mm to about 20.00 mm.

As seen in FIGS. 3A, 3B, 3C, and 3D, according to some embodiments, transfusion member 3 comprises a transfusion port 30. According to some embodiments, transfusion port 30 is port member 5b. According to some embodiments, transfusion port 30 encompasses transfusion passageway 13. According to some embodiments, transfusion port 30 has a first open end 30a and a second open end 30b. According to some embodiments, the first end of transfusion port 30a opens into the interior of the blood collection compartment 10, and the second end 30b opens into the exterior of the blood collection compartment. According to some embodiments, the interior of transfusion port 30 is sterile. According to some embodiments, the interior of transfusion port 30 is contiguous with, and extends from the interior of the blood collection compartment 10.

According to some embodiments, transfusion port 30 as a whole or any component thereof can be provided with particular features, such as, being formed with any suitable material or with additional components; and such as being formed in any suitable shape, length, width, and height to effect the rapid transfusion of blood or blood products within 8 minutes, within 7 minutes, within 6 minutes, within 5 minutes, within 4 minutes, within 3 minutes, within 2 minutes, within 1 minute.

According to some embodiments, transfusion port 30 can be configured with any internal diameter appropriate to rapidly transfuse blood. Examples of transfusion port 30 internal diameters can be seen in Table 13 appending this application. For example, the internal diameter of transfusion port 30 is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25 mm. For example, internal diameter of infusion port 20 is about 12.00 mm to about 20.00 mm.

According to some embodiments, transfusion port 30 is sealed so that it further comprises edges 4 comprising of a front edge 4a and a back edge, 4b wherein front edge 4a and back edge 4b are separably contiguous to create the seal. According to some such embodiments, front edge 4a and back edge 4b can be grasped and pulled apart or cut in order to unseal transfusion port 30. According to these embodiments, front edge 4a and back edge 4b can be grasped and pulled apart or cut in order to unseal the second end of transfusion port 30b. According to some embodiments, front edge 4a comprises a front tab 4a-i, and back edge 4b comprises a back tab 4b-i. According to some embodiments, front tab 4a-i and back tab 4b-i are configured to interdigitate.

According to some embodiments, transfusion port 30 further comprises at least one tubing fitting 5 and/or at least one tubing accessory 6. According to some embodiments, tubing fitting 5 is a membrane member 5c. According to some embodiments, membrane member 5c is configured to occlude the fluid communication in and out of transfusion port 30. According to some embodiments, tubing accessory 6 is a cap member 6c. According to some embodiments, cap member 6c is configured to cover the second end of transfusion port 30 to maintain its sterility. According to some embodiments, cap member 6c is configured to cover any one or more tubing fittings 5 that may be connected to the second end of transfusion port 30 to maintain sterility.

According to some embodiments, transfusion member 3 comprises a transfusion tube 31 that is an elongated structure with a hollow interior that allows for the outflow of blood from the blood collection compartment 10 through the transfusion port 30 via the transfusion passageway 13. According to some embodiments, transfusion tube 31 is tube member 5a.

According to some embodiments, transfusion tube 31 is separably or non-separably connected to transfusion port 30. According to some embodiments, transfusion tube 31 has a first end 31a and a second end 31b. According to some embodiments, first end of transfusion tube 31a is non-separably connected to a second end of infusion port 20b. According to some embodiments, the first end of transfusion tube 31a is nonseparably connected to the second end of transfusion port 30b by being integral with and extending from second end of transfusion port 30b. According to some embodiments, the interior of transfusion tube 31 is sterile and therefore maintains the sterility of the interior of blood compartment 10 and the interior of transfusion port 30. According to some embodiments, first end of transfusion tube 31a is separably connected to a second end of transfusion port 30b. According to some embodiments, the first end of transfusion tube 31 is separably connected to the second end of transfusion port 30b by penetrating into the second end of transfusion port 30b.

According to some embodiments, transfusion tube 31 further comprises at least one tubing fitting 5 and/or at least one tubing accessory 6. According to some embodiments, transfusion tube first end 31a comprises at least one tubing fitting 5 and/or at least one tubing accessory 6. According to some embodiments, transfusion tube second end 31b comprises at least one tubing fitting 5 and/or at least one tubing accessory 6. According to some embodiments, tubing fitting 5 is a penetrating member 5d. According to some embodiments, penetrating member 5d is configured to establish fluid communication between transfusion tube 31 and a second object or component, such as blood collection compartment 10 and/or the subject. According to some embodiments, tubing fitting 5 is a filter member 5e. According to some embodiments, filter member 5e is configured to filter fluid being fluidly communicated between transfusion tube 31 and a second object or component, such as blood collection compartment 10 and/or the subject. According to some embodiments, tubing fitting 5 is a coupling member 5f. According to some embodiments, coupling member 5f is configured to establish fluid communication between transfusion tube 31 and a second object or component, such as blood collection compartment 10 and/or the subject. According to some embodiments, tubing accessory 6 is a clamp member 6a. According to some embodiments, clamp member 6a is configured to occlude the fluid communication in and out of transfusion tube 31. According to some embodiments, tubing accessory 6 is a cap member 6c. According to some embodiments, cap member 6c is configured to cover the second end of transfusion tube 31 to maintain its sterility. According to some embodiments, cap member 6c is configured to cover any one or more tubing fittings 5 that may be connected to the second end of transfusion tube 31 to maintain sterility.

According to some embodiments, transfusion tube 31 as a whole or any component thereof can be provided with particular features, such as, being formed with any suitable material or with additional components; and such as being formed in any suitable shape, length, width, and height to effect the rapid transfusion of blood or blood products within 8 minutes, within 7 minutes, within 6 minutes, within 5 minutes, within 4 minutes, within 3 minutes, within 2 minutes, within 1 minute.

According to some embodiments, transfusion tube 31 may be of any length appropriate to accommodate the rapid transfusion of blood or blood products. Exemplary lengths can be seen in Table 7 appending this application. For example, transfusion tube 31 is about 175 mm, about 200 mm to about 450 mm in length. For example, transfusion tube 31 is about 200 mm, about 225 mm, about 250 mm, about 275 mm, about 300 mm, about 325 mm, about 350 mm, about 375 mm, about 400 mm, about 425 mm, or about 450 mm in length. For example, transfusion tube 31 is about 200 mm to about 300 mm in length.

According to some embodiments, transfusion tube 31 can have any inner diameter, outer diameter, and wall thickness sufficient to accommodate the rapid transfusion of blood or blood products. Example dimensions can be seen in Table 8a, Table 8b, Table 8c, and Table 8d appending this application. For example, the diameter of transfusion tube 31 is about 6.5 to about 25 mm, inclusive. For example, the internal diameter of transfusion tube 31 is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm in internal diameter. For example, internal diameter of transfusion tube 31 is about 12.00 mm to about 20.00 mm.

According to some embodiments, transfusion tube 31 can be formed from any material suitable that has high resistance to light, radiation, low temperatures, high temperatures moisture, pressure, abrasion, cracking, kinks, leaks, chemicals, fungal growth, bacterial growth; exhibits flexibly, tensile strength, or etc or any combination of the above. For example, transfusion tube 31 can be formed from polyvinyl chlorides (PVC), polyurethanes, thermoplastic poluerathanes (TPUs), polyvinylidene fluoride (PVF), polyethylene, ethylene vinyl acetates (EVAs) silicone, rubber, polypropelenes, fluorinated ethylene propylene (FEP), synthetics thereof (such as Teflon™, Kynar®, Silcryn™)

According to some embodiments, blood collection and autotransfusion container comprises more than one transfusion member 3. According to some embodiments, blood collection and autotransfusion container comprises at least two transfusion members. According to some embodiments, blood collection and autotransfusion container comprises at least three transfusion members. According to some embodiments, blood collection and autotransfusion container comprises at least four transfusion members. According to some embodiments, blood collection and autotransfusion container comprises at least five transfusion members.

### Kit

According to some embodiments, the described invention is a kit comprising:
i) a container component comprising a blood collection and autotransfusion container, wherein the blood collection and autotransfusion container comprises a blood container, an infusion member, and a transfusion member;
ii) a blood infusion component comprising a penetrating member, one or more clamp members, a connector member, one or more tube members, a luer member, optionally a chamber member, and optionally a filter member; and
iii) an instruction component describing the use of the blood collection and autotransfusion container;
wherein the contents of the kit container are sterile.

### Methods of Use

According to some embodiments, the described invention provides a method of rapidly collecting accumulated blood from a subject in need thereof and rapidly transfusing blood or blood products to the subject in need thereof

According to some embodiments, the method comprises the steps of:
a. providing a blood collection and autotransfusion container comprising:
   a blood container,
   an infusion member,
   and a transfusion member;
   wherein capacity of the blood container is at least one unit of blood;
   wherein the infusion member is connected to the blood container and the infusion member is configured to be in fluid communication with a subcutaneous drainage site on the subject and the blood container; and
   wherein the transfusion member connected to the blood container and the transfusion member is configured to be in fluid communication with the blood container and a subcutaneous transfusion site on the subject;
b. draining accumulated blood from the body cavity of a subject wherein the blood collection and autotransfusion container is connected to the subject via the infusion member;
c. collecting the blood in the blood collection and autotransfusion container by channeling the blood through the infusion member to a blood collection compartment in the blood container; and
d. transfusing blood or blood products to the subject by connecting the blood collection and autotransfusion container to the subcutaneous access site of the subject via the transfusion member; wherein the transfusion member is configured to be in fluid communication with a subcutaneous transfusion site on the subject and the blood collection compartment in the blood container.

According to some embodiments, the step of providing a blood collection and autotransfusion container further comprises providing more than one blood collection and autotransfusion container. According to some embodiments, if more than 1 blood collection and autotransfusion container is used for the collection of blood from a subject and the transfusion of blood or blood products to the subject, then the more than 1 blood collection and autotransfusion containers may be used concurrently and alternatively to effect the collection of blood from a subject and the transfusion of blood or blood products to the subject. For example, if 2 blood collection and autotransfusion containers are used then a first blood collection and autotransfusion container is used for the collection of blood from a subject and the transfusion of blood or blood products to the subject, and a second blood collection and autotransfusion container is used for the collection of blood from a subject while the first blood collection and autotransfusion container is transfusing the collected blood back to the subject; once the first blood collection and autotransfusion container is emptied and/or the second blood collection and autotransfusion container is filled then the second blood collection and autotransfusion container is used for the transfusion of blood or blood products to the subject, and if required, the first blood collection and autotransfusion container is used for the collection of blood from a subject while the second blood collection and autotransfusion container is transfusing the collected blood back to the subject. In another example, if 3 blood collection and autotransfusion containers are used then a first blood collection and autotransfusion container is used for the collection of blood from a subject and the transfusion of blood or blood products to the subject, and a second blood collection and autotransfusion container is used for the collection of blood from a subject while the first blood collection and autotransfusion container is transfusing the collected blood back to the subject, and a third blood collection and autotransfusion container is waiting on standby to be used; once the first blood collection and autotransfusion container is emptied and/or the second blood collection and autotransfusion container is filled then the second blood collection and autotransfusion container is used for the transfusion of blood or blood products to the subject, and the third blood collection and autotransfusion container is used for the collection of blood from a subject while the second blood collection and autotransfusion container is transfusing the collected blood back to the subject, and optionally the first blood collection and autotransfusion container is waiting on standby to be used. In another example, if 4 blood collection and autotransfusion containers are used then a first and second blood collection and autotransfusion containers are used for the collection of blood from a subject and the transfusion of blood or blood products to the subject, and a third and fourth blood collection and autotransfusion container is used for the collection of blood from a subject while the first blood collection and autotransfusion container is transfusing the collected blood back to the subject; once the first and second blood collection and autotransfusion containers are emptied and/or the third and fourth blood collection and autotransfusion containers are filled then the third and fourth blood collection and autotransfusion container is used for the transfusion of blood or blood products to the subject, and if required, the first and second blood collection and autotransfusion containers are used for the collection of blood from a subject while the third and fourth blood collection and autotransfusion containers are transfusing the collected blood back to the subject.

According to some embodiments, the step of providing a blood collection and autotransfusion container further comprises that the capacity providing a blood container configured with a capacity of one unit of blood, wherein one unit of blood is between about 25 mL and about 1500 mL. According to some embodiments, one unit of blood is about 500 mL, about 600 mL, about 700 mL, about 800 mL, about 900 mL, about 1000 mL, about 1100 mL, or about 1200 mL.

According to some embodiments, the step of providing a blood collection and autotransfusion container further comprises providing a blood container configured with a width between about 7 cm and about 30 cm dependent on its capacity.

According to some embodiments, the step of providing a blood collection and autotransfusion container further comprises providing a blood container configured with a length between about 9 cm and about 40 cm dependent on its capacity and its width.

According to some embodiments, the step of providing a blood collection and autotransfusion container further comprises providing the infusion member wherein the infusion member has an internal diameter of about 6.5 mm to about 25 mm, inclusive. For example, the internal diameter of the infusion member is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25 mm. For example, the internal diameter of the infusion member is about 12.00 mm to about 20.00 mm, inclusive, i.e., about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm.

According to some embodiments, the step of providing a blood collection and autotransfusion container further comprises providing the transfusion member wherein the transfusion member is configured with an internal diameter of about 6.5 mm to about 25 mm, inclusive. For example, internal diameter of the transfusion member is about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25 mm. For example, internal diameter of the transfusion is about 12.00 mm to about 20.00 mm, i.e., about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm

According to some embodiments, the step of providing a blood collection and autotransfusion container further comprises providing one or more tubing fittings. According to some embodiments, the step of providing a blood collection and autotransfusion container further comprises providing one or more tubing accessories. According to some embodiments, the step of providing a blood collection and autotransfusion container further comprises providing one or more tubing accessories and one or more tubing fittings. According to some embodiments, the step of providing a blood collection and autotransfusion container further comprises providing one or more tubing accessories or one or more tubing fittings.

According to some embodiments, the one or more tubing fittings comprises a port member, tube member, membrane member, connector/coupler member, filter member, valve member, and/or a penetrating member, and/or any combination thereof. According to some embodiments, the one or more tubing fittings comprises one or more port members, one or more tube members, one or more membrane member, one or more connector/coupler members, one or more filter members, one or more valve members, and/or one or more penetrating members, and/or any combination thereof. According to some embodiments, the one or more tubing fittings comprises one or more port members and one or more tube members. According to some embodiments, the one or more tubing fittings comprises one or more port members, one or more tube members, and one or more membrane members. According to some embodiments, the one or more tubing fittings comprises one or more port members, one or more tube members, one or more member members, and one or more connector/coupler members. According to some embodiments, the one or more tubing fittings comprises one or more port members, one or more tube members, one or more membrane members, one or more connector/coupler members, and one or more filter members. According to some embodiments, the one or more tubing fittings comprises one or more port members, one or more tube members, one or more membrane members, one or more connector/coupler members, one or more filter members and one or more valve members. According to some embodiments, the one or more tubing fittings comprises one or more port members, one or more tube members, one or more membrane members, one or more connector/coupler members, one or more filter members, one or more valve members, and one or more penetrating members.

According to some embodiments, the one or more tubing accessories comprise a hanging member, a cap member, and/or a clamp member and/or any combination thereof. According to some embodiments, the one or more tubing accessories comprise one or more hanging members, one or more cap members, and/or one or more clamp members and/or any combination thereof. According to some embodiments, the one or more tubing accessories comprise one or more hanging members and one or more cap members. According to some embodiments, the one or more tubing accessories comprise one or more hanging members, one or more cap members, and one or more clamp members.

According to some embodiments, the tubing fitting comprises a port member. According to some embodiments, the internal diameter of the port member is about 6.5 mm to about 25 mm, inclusive. According to some embodiments, the internal diameter of the port member is about 12.00 mm to about 20.00 mm, inclusive.

According to some embodiments, the tubing fitting comprises a tube member. According to some embodiments, the internal diameter of the tube member is about 6.5 mm to about 25 mm, inclusive. According to some embodiments, the internal diameter of the tube member is about 12.00 mm to about 20.00 mm, inclusive. According to some embodiments, the length of the tube member is about 175 mm to about 450 mm, inclusive. According to some embodiments, the length of the tube member is about 200 mm to about 300 mm, inclusive.

According to some embodiments, the tubing fitting comprises a filter member. According to some embodiments, the filter member comprises a filter with a pore size of about 170 microns to about 260 microns.

According to some embodiments, the tubing fitting comprises a penetrating member. According to some embodiments, the inner diameter of the penetrating member is about 0.003 in to about 1.083 in, inclusive. According to some embodiments, the inner diameter of the penetrating member is about 0.0625 in to about 0.333 in, inclusive. According to some embodiments, the gauge of penetrating member is about 3 Fr to about 40 Fr, inclusive. According to some embodiments, the gauge of penetrating member is about 8 Fr to about 36 Fr, inclusive. According to some embodiments, the gauge of penetrating member is about 14 Fr to about 24 Fr, inclusive.

According to some embodiments the tubing accessory comprises a hanging member. According to some embodiments the tubing accessory comprises a clamp member. According to some embodiments, the tubing accessory comprises cap member.

According to some embodiments, the step of providing a blood collection and autotransfusion container further comprises providing one or more tubing fittings, wherein the first tubing fitting connects to the second tubing fitting. According to some embodiments, the step of providing a blood collection and autotransfusion container further comprises providing one or more tubing accessories and one or more tubing fittings, wherein the one or more tubing accessories attaches to the one or more tubing fitting. According to some embodiments, the step of providing a blood collection and autotransfusion container further comprises providing one or more tubing fittings wherein the first tubing fitting connects to the second tubing fitting and the one or more tubing accessories attaches to the first tubing fitting or the second tubing fitting.

According to some embodiments, the step of providing a blood collection and autotransfusion container further comprises providing one or more tubing fittings wherein the one or more tubing fittings comprise a tube member and a penetrating member, and the tube member is connected to the penetrating member. According to some embodiments, the step of providing a blood collection and autotransfusion container further comprises providing one or more tubing accessories and one or more tubing fittings, wherein the one or more tubing accessories comprises a clamp member and the one or more tubing fitting comprises a tube member, wherein the clamp member is attached to the tube member. According to some embodiments, the step of providing a blood collection and autotransfusion container further comprises providing one or more tubing accessories and one or more tubing fittings, wherein the one or more tubing accessories comprises a cap member and the one or more tubing fitting comprises a penetrating member wherein the cap member is attached to the penetrating member.

According to some embodiments, the step of providing a blood collection and autotransfusion container further comprises providing one or more tubing accessories and one or more tubing fittings, wherein the one or more tubing accessories comprises a clamp member and a cap member, and the one or more tubing fitting, comprise a tube member and a penetrating member, wherein the penetrating member is connected to the tube member, the clamp member is attached to the tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a clamp member and a cap member and the one or more tubing fitting comprises a tube member, a penetrating member, and a filter member, wherein the penetrating member is connected to the tube member, the tube member is connected to the filter member, the clamp member is attached to the tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a first clamp member, a second clamp member, and a cap member, and the one or more tubing fittings comprises a first tube member, a second tube member, a penetrating member, and a filter member, wherein the penetrating member is connected to the first tube member, the first tube member is connected to the filter member, the filter member is connected to a second tube member, the first clamp member is attached to the first tube member, the second clamp member is attached to the second tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a first clamp member, a second clamp member, and a cap member and the one or more tubing fittings comprises a first tube member, a second tube member, a penetrating member, a filter member, and a port member, wherein the penetrating member is connected to the first tube member, the first tube member is connected to the filter member, the filter member is connected to a second tube member, the second tube member is attached to the port member, the first clamp member is attached to the first tube member, the second clamp member is attached to the second tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a first clamp member, a second clamp member, and a cap member and the one or more tubing fittings comprises a first tube member, a second tube member, a third tube member, a penetrating member, a filter member, and a port member wherein the penetrating member is connected to the first tube member, the first tube member is connected to the filter member, the filter member is connected to a second tube member, the second tube member is attached to the port member, the port member is attached to the third tube member, the first clamp member is attached to the first tube member, the second clamp member is attached to the second tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a first clamp member, a second clamp member, and a cap member and the one or more tubing fittings comprises a first tube member, a second tube member, a third tube member, a penetrating member, a filter member, a port member, and a connector member, wherein the penetrating member is connected to the first tube member, the first tube member is connected to the filter member, the filter member is connected to a second tube member, the second tube member is attached to the port member, the port member is attached to the third tube member, the third tube member is attached to the connector member, the first clamp member is attached to the first tube member, the second clamp member is attached to the second tube member, and the cap member is attached to the penetrating member.

According to some embodiments, the step of providing a blood collection and autotransfusion container further comprises providing one or more tubing accessories and one or more tubing fittings, wherein the one or more tubing accessories comprises a first clamp member, a second clamp member, a first cap member, and a second cap member and the one or more tubing fittings comprises a first tube member, a second tube member, a third tube member, a first penetrating member, a second penetrating member a filter member, a port member, and a connector member, wherein the first penetrating member is connected to the first tube member, the first tube member is connected to the filter member, the filter member is connected to a second tube member, the second tube member is attached to the port member, the port member is attached to the third tube member, the third tube member is attached to the connector member, the connector member is attached to the second penetrating member, the first clamp member is attached to the first tube member, the second clamp member is attached to the second tube member, the first cap member is attached to the first penetrating member, and the second cap member is attached to the second penetrating member.

According to some embodiments, the step of providing a blood collection and autotransfusion container further comprises providing one or more tubing fittings and connecting one or more tubing fittings to the blood container, infusion member, and/or transfusion member, wherein the tubing fitting is configured to be in fluid communication with the blood container, infusion member, and/or transfusion member, or an component thereof. According to some embodiments, the step of providing a blood collection and autotransfusion container further comprises connecting one or more tubing fittings to the blood container and/or any component thereof, wherein the tubing fitting is configured to be in fluid communication with the blood container. According to some embodiments, the step of providing a blood collection and autotransfusion container further comprises connecting one or more tubing fittings to the infusion member and/or any component thereof, wherein the tubing fitting is configured to be in fluid communication with the infusion member. According to some embodiments, the step of providing a blood collection and autotransfusion container further comprises connecting one or more tubing fittings to the transfusion member and/or any component thereof, wherein the tubing fitting is configured to be in fluid communication with the transfusion member.

According to some embodiments, the step of providing a blood collection and autotransfusion container further comprises providing one or more tubing fittings and connecting the one or more tubing fittings to the blood container, infusion member, and/or transfusion member, or any connected component thereof, such as a tubing fitting, wherein the one or more tubing fittings are configured to be in fluid communication with the blood container, infusion member, and/or transfusion member, or any component thereof. For example, the step of providing a blood collection and autotransfusion container further comprises connecting one or more tubing fittings to the infusion member, wherein the one or more tubing fittings comprises a port member and a membrane member and the infusion member is connected to the port member, and the port member is connected membrane member. For example, the step of providing a blood collection and autotransfusion container further comprises connecting one or more tubing fittings to the transfusion member, wherein the one or more tubing fittings comprises a tube member and a penetrating member, and the transfusion member is connected to the tube member, and the tube member is connected to the penetrating member. For example, the step of providing a blood collection and autotransfusion container further comprises connecting one or more tubing fittings to the infusion member, wherein the one or more tubing fittings comprises a tube member and a penetrating member, and the infusion member is connected to the tube member, and the tube member is connected to the penetrating member. For example, the step of providing a blood collection and autotransfusion container further comprises connecting one or more tubing fittings to the transfusion member, wherein the one or more tubing fittings comprises a port member and a membrane member, and the transfusion member is connected to the port member and the port member is connected membrane member.

According to some embodiments, the step of providing a blood collection and autotransfusion container further comprises providing one or more tubing accessory and attaching one or more tubing accessories to the blood container, infusion member, transfusion member, or any component thereof, such as any connected tubing fitting thereof.

According to some embodiments, the step of providing a blood collection and autotransfusion container further comprises providing one or more tubing accessories and attaching the one or more tubing accessories to the blood container. According to some embodiments, the step of providing a blood collection and autotransfusion container further comprises attaching one or more tubing accessories to the blood container, wherein the tubing accessory comprises a hanging member.

According to some embodiments, the step of providing a blood collection and autotransfusion container further comprises attaching one or more tubing accessories to the infusion member. According to some embodiments, the step of providing a blood collection and autotransfusion container further comprises attaching one or more tubing accessories to the infusion member, wherein the tubing accessory comprises a cap member or a clamp member. According to some embodiments, the step of providing a blood collection and autotransfusion container further comprises attaching one or more tubing accessories to the transfusion member, wherein the tubing accessory comprises a cap member or a clamp member.

According to some embodiments, the subcutaneous drainage site comprises a drainage tube inserted into the body cavity of the subject containing the accumulated blood and wherein the drainage tube is in fluid communication with the infusion member. According to some embodiments, the step of draining accumulated blood from the body cavity of a subject comprises inserting the drainage tube in the body cavity of the subject containing the accumulated blood, and connecting the drainage tube to the infusion member of the blood collection and autotransfusion container.

According to some embodiments, the step of draining accumulated blood from the body cavity of a subject further comprises regulating the pressure differential resulting from draining accumulated blood from a subject by providing a pressure regulator wherein the pressure regulator is connected to the subcutaneous drainage site and the infusion member of the blood collection and the autotransfusion device. According to some embodiments, the pressure regulator comprises a chest drainage device or a gas vent.

According to some embodiments, the step of draining accumulated blood from the body cavity of a subject further comprises forcing the accumulated blood to be drained from a subject wherein the force comprises a gravitational force or a suction force. According to some embodiments, forcing the accumulated blood to be drained from a subject by a gravitational force comprises configuring the spatial arrangement of the blood collection and autotransfusion container to be at a lower height than the body cavity of the subject. According to some embodiments, forcing the accumulated blood to be drained from a subject by a gravitational force comprises configuring the spatial arrangement of the chest drainage device to be at a lower height than the body cavity of the subject and the blood container of the blood collection and autotransfusion container to be at a lower height than the chest drainage device. According to some embodiments, forcing the accumulated blood to be drained from a subject by a suction force comprises connecting a suction tube with suction action to the blood container of the blood collection and autotransfusion container and activating the suction action. According to some embodiments, forcing the accumulated blood to be drained from a subject by a suction force comprises connecting a suction tube with suction action to the chest drainage device and activating the suction action.

According to some embodiments, the step of draining accumulated blood from the body cavity of a subject comprises connecting the subcutaneous drainage site to the infusion member of the blood collection and autotransfusion container, and forcing the accumulated blood to be drained from a subject by arranging the spatial configuration of the pressure regulator and the blood container of the blood collection and autotransfusion device or by connecting a suction force to the pressure regulator and activating the suction force. According to some embodiments, the step of draining accumulated blood from the body cavity of a subject comprises connecting the subcutaneous drainage site to a pressure regulator, connecting the pressure regulator to infusion member of the blood collection and autotransfusion container, and forcing the accumulated blood to be drained from a subject by arranging the spatial configuration of the pressure regulator and the blood container of the blood collection and autotransfusion device or by connecting a suction force to the pressure regulator and activating the suction force.

According to some embodiments, the step of collecting the blood comprises connecting the subcutaneous drainage site to the infusion member of the blood collection and autotransfusion container and forcing the blood to be channeled to the blood collection and autotransfusion container, wherein the force comprises a gravitational force. According to some embodiments, forcing the blood to be channeled to the blood collection and autotransfusion container by gravitational force comprises arranging the spatial configuration of the blood container of the blood collection and autotransfusion device to be at a lower height than the blood.

According to some embodiments, the step of transfusing the collected blood back to the subject comprises forcing the collected blood to be transfused to the subject through the transfusion member wherein the force comprises a compression force, gravitational force, or by a pressurized pump. According to some embodiments, forcing the collected blood to be transfused to the subject through the transfusion member by a compression force comprises attaching the blood container of the blood collection and autotransfusion container in a compression chamber with compression action and activating the compression action. According to some embodiments, forcing the collected blood to be transfused to the subject through the transfusion member by a gravitational force comprises configuring the spatial arrangement of the blood container to be at a higher height than the subcutaneous transfusion site on the subject. According to some embodiments, forcing the collected blood to be transfused to the subject through the transfusion member by a pressurized pump comprises attaching a pressurized pump to the blood container of the blood collection and autotransfusion container activating the pressurizing action.

According to some embodiments, the step of transfusing the collected blood back to the subject comprises warming the collected blood by connecting a blood warmer to the transfusion member wherein the transfusion member and the blood warmer are in fluid communication with the subcutaneous transfusion site of the subject.

According to some embodiments, the step of transfusing the collected blood back to the subject comprises filtering the collected blood by connecting a blood filter to the transfusion member wherein the transfusion member and the blood filter are in fluid communication with the subcutaneous transfusion site of the subject. According to some embodiments, the step of transfusing the collected blood back to the subject comprises filtering the collected blood by connecting a blood filter to a blood warmer connected to the transfusion member wherein the transfusion member and the blood filter is in fluid communication with the subcutaneous transfusion site on the subject.

According to some embodiments, the step of transfusing the collected blood back to the subject comprises releasing gas from the collected blood by connecting a gas vent to the transfusion member wherein the transfusion member and the gas vent are in fluid communication with the subcutaneous transfusion site on a subject. According to some embodiments, the step of transfusing the collected blood back to the subject comprises releasing gas from the collected blood by connecting a gas vent to the blood warmer connected to the transfusion member wherein the transfusion member and the gas vent are in fluid communication with the subcutaneous transfusion site on a subject. According to some embodiments, the step of transfusing the collected blood back to the subject comprises releasing gas from the collected blood by connecting a gas vent to the blood filter connected to the transfusion member wherein the transfusion member and the gas vent are in fluid communication with the subcutaneous transfusion site on a subject. According to some embodiments, the step of transfusing the collected blood back to the subject comprises releasing gas from the collected blood by connecting a gas vent to the blood filter connected to blood warmer connected to the transfusion member wherein the transfusion member and the gas vent are in fluid communication with the subcutaneous transfusion site on a subject. According to some embodiments, the step of transfusing the collected blood back to the subject comprises releasing gas from the collected blood by connecting a gas vent to the blood warmer connected to blood filter connected to the transfusion member wherein the transfusion member and the gas vent are in fluid communication with the subcutaneous transfusion site on a subject.

According to some embodiments, the step of transfusing the collected blood back to the subject comprises forcing the collected blood in the blood container to be transfused to the subject and transfusing the blood by connecting the transfusion member to the subcutaneous transfusion site on the subject. According to some embodiments, the step of transfusing the collected blood back to the subject comprises forcing the collected blood in the blood container to be transfused to be patient, warming the forced blood by connecting a blood warmer to the transfusion member, and transfusing the blood by connecting the blood warmer to the subcutaneous transfusion site on the subject. According to some embodiments, the step of transfusing the collected blood back to the subject comprises forcing the collected blood in the blood container to be transfused to be patient, filtering the forced blood by connecting a blood filter to the transfusion member, and transfusing the blood by connecting the blood warmer to the subcutaneous transfusion site on the subject.

According to some embodiments, the step of transfusing the collected blood back to the subject comprises connecting the transfusion member to a subcutaneous transfusion site on the subject, and transfusing the collected blood through the transfusion member into the subject. According to some embodiments, the step of transfusing the collected blood back to the subject comprises connecting the transfusion member to a blood warmer, connecting the blood warmer to the subcutaneous transfusion site on the subject, and transfusing the collected blood through the transfusion member and the blood warmer into the subject. According to some embodiments, the step of transfusing the collected blood back to the subject comprises connecting the transfusion member to a blood filter, connecting the blood filter to the subcutaneous transfusion site on the subject, and transfusing the collected blood through the transfusion member and the blood filter into the subject. According to some embodiments, the step of transfusing the collected blood back to the subject comprises connecting the transfusion member to a gas vent, connecting the gas vent to the subcutaneous transfusion site on the subject, and transfusing the collected blood through the transfusion member and the gas vent into the subject. According to some embodiments, the step of transfusing the collected blood back to the subject comprises connecting the transfusion member to a blood filter, connecting the blood filter to a gas vent, connecting the gas vent to the subcutaneous transfusion site on the subject, and transfusing the collected blood through the transfusion member, blood filter, and gas vent into the subject. According to some embodiments, the step of transfusing the collected blood back to the subject comprises connecting the transfusion member to a gas vent, connecting the gas vent to a blood filter, connecting the blood filter to the subcutaneous transfusion site on the subject, and transfusing the collected blood through the transfusion member, gas vent, and blood filter into the subject. According to some embodiments, the step of transfusing the collected blood back to the subject comprises connecting the transfusion member to a blood filter, connecting the blood filter to a blood warmer, connecting the blood warmer to the subcutaneous transfusion site on the subject, and transfusing the collected blood through the transfusion member, blood filter, and blood warmer into the subject. According to some embodiments, the step of transfusing the collected blood back to the subject comprises connecting the transfusion member to a blood warmer, connecting the blood warmer to a blood filter, connecting the blood filter to the subcutaneous transfusion site on the subject, and transfusing the collected blood through the transfusion member, blood warmer, and blood filter into the subject. According to some embodiments, the step of transfusing the collected blood back to the subject comprises connecting the transfusion member to a blood warmer, connecting the blood warmer to a gas vent, connecting the gas vent to the subcutaneous transfusion site on the subject, and transfusing the collected blood through the transfusion member, blood warmer, and gas vent into the subject. According to some embodiments, the step of transfusing the collected blood back to the subject comprises connecting the transfusion member to a gas vent, connecting the gas vent to a blood warmer, connecting the blood warmer to the subcutaneous transfusion site on the subject, and transfusing the collected blood through the transfusion member, gas vent, and blood warmer into the subject. According to some embodiments, the step of transfusing the collected blood back to the subject comprises connecting the transfusion member to a blood filter, connecting the blood filter to a blood warmer, connecting the blood warmer to a gas vent, connecting the gas vent to the subcutaneous transfusion site on the subject and transfusing the collected blood through the transfusion member, blood filter, blood warmer, and gas vent into the subject. According to some embodiments, the step of transfusing the collected blood back to the subject comprises connecting the transfusion member to a blood warmer, connecting the blood warmer to a gas vent, connecting the gas vent to a blood filter, connecting the blood filter to the subcutaneous transfusion site on the subject, and transfusing the collected blood through the transfusion member, blood warmer, gas vent, and blood filter into the subject. According to some embodiments, the step of transfusing the collected blood back to the subject comprises connecting the transfusion member to a gas vent, connecting the gas vent to a blood filter, connecting the blood filter to a blood warmer, connecting the blood warmer to the subcutaneous transfusion site on the subject, and transfusing the collected blood through the transfusion member, gas vent, blood filter, and blood warmer into the subject. According to some embodiments, the step of transfusing the collected blood back to the subject comprises connecting the transfusion member to a gas vent, connecting the gas vent to a blood warmer, connecting the blood warmer to a blood filter, connecting the blood filter to the subcutaneous transfusion site on the subject, and transfusing the collected blood through the transfusion member, gas vent, blood warmer, and blood filter into the subject. According to some embodiments, the step of transfusing the collected blood back to the subject comprises connecting the transfusion member to a blood warmer, connecting the blood warmer to a gas vent, connecting the gas vent to a blood filter, connecting the blood filter to the subcutaneous transfusion site on the subject, and transfusing the collected blood through the transfusion member, blood warmer, gas vent, and blood filter into the subject. According to some embodiments, the step of transfusing the collected blood back to the subject comprises connecting the transfusion member to a blood warmer, connecting the blood warmer to a blood filter, connecting the blood filter to a gas vent, connecting the gas vent to the subcutaneous transfusion site on the subject, and transfusing the collected blood through the transfusion member, blood warmer, blood filter, and gas vent into the subject.

According to some embodiments, the step of transfusing the collected blood back to the subject comprises connecting the transfusion member to a subcutaneous transfusion site on the subject, and forcing the collected blood to be transfused to the subject through the transfusion member into the subject. According to some embodiments, the step of transfusing the collected blood back to the subject comprises connecting the transfusion member to a blood warmer, connecting the blood warmer to the subcutaneous transfusion site on the subject, and forcing the collected blood to be transfused to the subject through the transfusion member and the blood warmer into the subject. According to some embodiments, the step of transfusing the collected blood back to the subject comprises connecting the transfusion member to a blood filter, connecting the blood filter to the subcutaneous transfusion site on the subject, and forcing the collected blood to be transfused to the subject through the transfusion member and the blood filter into the subject. According to some embodiments, the step of transfusing the collected blood back to the subject comprises connecting the transfusion member to a gas vent, connecting the gas vent to the subcutaneous transfusion site on the subject, and forcing the collected blood to be transfused to the subject through the transfusion member and the gas vent into the subject. According to some embodiments, the step of transfusing the collected blood back to the subject comprises connecting the transfusion member to a blood filter, connecting the blood filter to a gas vent, connecting the gas vent to the subcutaneous transfusion site on the subject, and forcing the collected blood to be transfused to the subject through the transfusion member, blood filter, and gas vent into the subject. According to some embodiments, the step of transfusing the collected blood back to the subject comprises connecting the transfusion member to a gas vent, connecting the gas vent to a blood filter, connecting the blood filter to the subcutaneous transfusion site on the subject, and forcing the collected blood to be transfused to the subject through the transfusion member, gas vent, and blood filter into the subject. According to some embodiments, the step of transfusing the collected blood back to the subject comprises connecting the transfusion member to a blood filter, connecting the blood filter to a blood warmer, connecting the blood warmer to the subcutaneous transfusion site on the subject, and forcing the collected blood to be transfused to the subject through the transfusion member, blood filter, and blood warmer into the subject. According to some embodiments, the step of transfusing the collected blood back to the subject comprises connecting the transfusion member to a blood warmer, connecting the blood warmer to a blood filter, connecting the blood filter to the subcutaneous transfusion site on the subject, and forcing the collected blood to be transfused to the subject through the transfusion member, blood warmer, and blood filter into the subject. According to some embodiments, the step of transfusing the collected blood back to the subject comprises connecting the transfusion member to a blood warmer, connecting the blood warmer to a gas vent, connecting the gas vent to the subcutaneous transfusion site on the subject, and forcing the collected blood to be transfused to the subject through the transfusion member, blood warmer, and gas vent into the subject. According to some embodiments, the step of transfusing the collected blood back to the subject comprises connecting the transfusion member to a gas vent, connecting the gas vent to a blood warmer, connecting the blood warmer to the subcutaneous transfusion site on the subject, and forcing the collected blood to be transfused to the subject through the transfusion member, gas vent, and blood warmer into the subject. According to some embodiments, the step of transfusing the collected blood back to the subject comprises connecting the transfusion member to a blood filter, connecting the blood filter to a blood warmer, connecting the blood warmer to a gas vent, connecting the gas vent to the subcutaneous transfusion site on the subject, and forcing the collected blood to be transfused to the subject through the transfusion member, blood filter, blood warmer, and gas vent into the subject. According to some embodiments, the step of transfusing the collected blood back to the subject comprises connecting the transfusion member to a blood warmer, connecting the blood warmer to a gas vent, connecting the gas vent to a blood filter, connecting the blood filter to the subcutaneous transfusion site on the subject, and forcing the collected blood to be transfused to the subject through the transfusion member, blood warmer, gas vent, and blood filter into the subject. According to some embodiments, the step of transfusing the collected blood back to the subject comprises connecting the transfusion member to a gas vent, connecting the gas vent to a blood filter, connecting the blood filter to a blood warmer, connecting the blood warmer to the subcutaneous transfusion site on the subject, and forcing the collected blood to be transfused to the subject through the transfusion member, gas vent, blood filter, and blood warmer into the subject. According to some embodiments, the step of transfusing the collected blood back to the subject comprises connecting the transfusion member to a gas vent, connecting the gas vent to a blood warmer, connecting the blood warmer to a blood filter, connecting the blood filter to the subcutaneous transfusion site on the subject, and forcing the collected blood to be transfused to the subject through the transfusion member, gas vent, blood warmer, and blood filter into the subject. According to some embodiments, the step of transfusing the collected blood back to the subject comprises connecting the transfusion member to a blood warmer, connecting the blood warmer to a gas vent, connecting the gas vent to a blood filter, connecting the blood filter to the subcutaneous transfusion site on the subject, and forcing the collected blood to be transfused to the subject through the transfusion member, blood warmer, gas vent, and blood filter into the subject. According to some embodiments, the step of transfusing the collected blood back to the subject comprises connecting the transfusion member to a blood warmer, connecting the blood warmer to a blood filter, connecting the blood filter to a gas vent, connecting the gas vent to the subcutaneous transfusion site on the subject, and forcing the collected blood to be transfused to the subject through the transfusion member, blood warmer, blood filter, and gas vent into the subject.

According to some embodiments, the subcutaneous transfusion site on the subject comprises a vascular access device inserted into the vascular system of a subject, wherein the vascular access device is in fluid communication with the transfusion member. According to some embodiments, the step of transfusing the collected blood back to the subject comprises inserting the vascular access device into the vascular system of a subject, and connecting the vascular access device to the transfusion member. According to some embodiments, the step of transfusing the collected blood back to the subject comprises inserting the vascular access device into the vascular system of a subject, and connecting the vascular access device to the blood warmer. According to some embodiments, the step of transfusing the collected blood back to the subject comprises inserting the vascular access device into the vascular system of a subject, and connecting the vascular access device to the blood filter. According to some embodiments, the step of transfusing the collected blood back to the subject comprises inserting the vascular access device into the vascular system of a subject, and connecting the vascular access device to the gas vent.

According to some embodiments, the subcutaneous drainage site comprises a drainage tube inserted into the body cavity of the subject containing the accumulated blood and wherein the drainage tube is in fluid communication with the infusion member. According to some embodiments, the step of draining accumulated blood from the body cavity of a subject comprises inserting the drainage tube in the body cavity of the subject containing the accumulated blood, and connecting the drainage tube to the infusion member of the blood collection and autotransfusion container.

According to some embodiments, the blood collection and autotransfusion container, and any component thereof can be connected by one or more tubing fittings that allows for fluid communication between two or more components. According to some embodiments, a tubing fitting can be a port member, tube member, connector/coupler member, filter member, valve member, or a penetrating member.

For example, the infusion member is in fluid communication with the subcutaneous transfusion site by connecting to a tube member connected to the pressure regulator and/or the subcutaneous drainage site. In another example, the infusion member is in fluid communication with the subcutaneous transfusion site by connecting to a penetrating member that is connected to a tube member that is connected to the pressure regulator and/or the subcutaneous drainage site. In another example, the infusion member is in fluid communication with the subcutaneous transfusion site by connecting to a tube member that is connected to a penetrating member that is connected to a tube member that is connected to the pressure regulator and/or the subcutaneous drainage site.

In another example, the transfusion member is in fluid communication with the subcutaneous transfusion site by connecting to a tube member that is connected to the blood filter, blood warmer, gas vent or subcutaneous transfusion site. In another example, the transfusion member is in fluid communication with the subcutaneous transfusion site by connecting to a tube member connected to a penetrating member connected to a tube member connected to the blood filter, blood warmer, gas vent, and/or subcutaneous transfusion site. In another example, the transfusion member is in fluid communication with the subcutaneous transfusion site by connecting to a tube member connected to a connector member connected to a tube member connected to the blood filter, blood warmer, gas vent, and/or subcutaneous transfusion site. In another example, the transfusion member is in fluid communication with the subcutaneous transfusion site by connecting to a penetrating member that is connected to a tube member that is connected to a connecter member that is connected to a tube member connected to the blood filter, blood warmer, gas vent, and/or subcutaneous transfusion site.

According to some embodiments, a tubing attachment may be attached to the blood collection and autotransfusion container, or any component thereof, and/or tubing fitting, or any component thereof. According to some embodiments, a tubing accessory can be a clamp member, a hanging member, or a cap member. For example, a clamp member may be attached to the infusion member or the transfusion member. In another example, a clamp member may be attached to a tube member.

According to some embodiments, the described invention provides an blood collection and autotransfusion container configured for the treatment of a subject undergoing hemorrhagic shock. According to some embodiments, the described blood collection and autotransfusion container is configured for the treatment of a subject undergoing Class I, Class II, Class III, or Class IV hemorrhagic shock. According to some embodiments, the described blood collection and autotransfusion container is configured for the treatment of a subject undergoing Class III, or Class IV hemorrhagic shock.

According to some embodiments, the described invention provides a method of rapidly collecting a unit of blood from the body cavity of a subject, wherein the standard measure of a unit of blood is 600 cc. According to some embodiments, the described invention provides a method of rapidly collecting a unit of blood from the body cavity of a subject in less than 8 minutes, less than 7 minutes, less than 6 minutes, less than 5 minutes, less than 4 minutes, less than 3 minutes, less than 2 minutes, or less than 1 minute.

According to some embodiments, the described invention provides a method of rapidly transfusing a unit of blood. According to some embodiments, the described invention provides a method of rapidly transfusing one unit of blood in less than 5 minutes, less than 4 minutes, less than 3 minutes, less than 2 minutes, or less than 1 minute.

According to some embodiments, the described invention provides a method of rapidly collecting from a patient and rapidly transfusing blood a unit of blood back to the patient. According to some embodiments, the described invention provides a method of rapidly collecting and rapidly transfusing blood a unit of blood in less than 10 minutes, less than 9 minutes, less than 8 minutes, less than 7 minutes, less than 6 minutes, less than 5 minutes, less than 4 minutes, less than 3 minutes, less than 2 minutes, or less than 1 minute

According to some embodiments, the described invention provides a method of rapidly, and safely collecting and transfusing blood or blood products to a subject in need thereof in a variety of settings, such as in emergency and/or trauma care in the field, on site, or in a hospital setting, pre-operative, intra-operative, and post-operative, intensive-care setting, in-patient, out-patient, and the like. According to some embodiments, the method described herein are useable by thoracic surgeons, cardiac surgeons, gynecological surgeons, general surgeons, pulmonary physicians, oncologists, critical care physicians, nurses, home health care professionals, and other health care professionals.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, exemplary methods and materials have been described.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety to disclose and described the methods and/or materials in connection with which the publications are cited. The dates of publication provided may be different from the actual publication dates which may need to be independently confirmed.

### EXAMPLES

The following example is put forth so as to provide those of ordinary skill in the art with a complete disclosure and description of how to make and use the present invention, and is not intended to limit the scope of what the inventors regard as their invention nor is it intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

A 1000 mL blood container will be produced with commercially available polyvinyl chloride plasticizer-free formulation, sterilized according to industry standards, and designed to substantially resemble FIGS. 3A and 3B with an infusion port positioned at the top-right of the blood container, and a transfusion tube positioned at the bottom-center of the container. The infusion port will have an internal diameter of about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm and the transfusion tube will have an internal diameter of about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, and about 20.0 mm.

A drainage tube will be inserted into a patient with bleeding into the pleural cavity of the patient. A blood drainage device (e.g. a PLEUR-EVAC®) will be connected to the chest tube through its drained blood tube and will be utilized to regulate the pressure differential and drain the blood from the patient. The drained blood will be channeled to the 1000 mL container by connecting the infusion port of the container to a standard blood infusion kit (e.g., BD AlarisTM Pump Infusion Blood Set) via a container spike and by connecting the blood infusion kit to the blood chamber outflow tube of the drainage device. The blood flow rate will allow drainage to collection to take about 2-7 minutes. Once the 1000 mL container is filled, the spike will be removed quickly from the infusion port. The 1000 mL container will then be transferred to an automatic blood transfuser (e.g. a Level 1® H-1200 Fast Flow Fluid Warmer) where the container will be placed in the compression chamber. A second blood infusion kit will be used to connect the transfusion tube of the 1000 mL container to a blood warmer, a blood filter or both, and to the patient so that the blood is transferred back to the patient.

Blood flow rate will be such that time from collection to transfusion will be will be less than about 10 minutes, in less than about 9 minutes, in less than about 8 minutes, less than about 7 minutes, less than about 6 minutes, less than about 5 minutes, less than about 4 minutes, or less than about 3 minutes.

### Example 2

A 1000 mL blood container will be produced with a commercially available polyvinyl chloride plasticizer-free formulation, sterilized according to industry standards, and designed to substantially resemble FIGS. 3C and 3D with an infusion tube positioned along the top-right of the blood container, and a transfusion port positioned along the bottom-center of the container. The infusion tube will have an internal diameter of about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, and about 20.0 mm and the transfusion port will have an internal diameter of 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm.

The drainage tube will be inserted into a patient with bleeding into the pleural cavity of the patient. A blood drainage device (e.g. a PLEUR-EVAC®) will be connected to the chest tube through its drained blood tube and will be utilized to regulate the pressure differential and drain the blood from the patient. The drained blood will be channeled to the 1000 mL container by connecting the infusion tube of the container to a standard blood infusion kit (e.g., BD Alaris™ Pump Infusion Blood Set) via a container spike and by connecting the blood infusion kit to the blood chamber outflow tube of the drainage device. The blood flow rate will allow drainage to collection to take about 2-7 minutes. Once the 1000 mL container is filled, the infusion tube will be clamped and the spike will be quickly removed from the standard blood infusion kit. The 1000 mL container will then be transferred to an automatic blood transfuser (e.g. a Level 1® H-1200 Fast Flow Fluid Warmer) where the container will be placed in the compression chamber. A second blood infusion kit will be used to spike the transfusion port and connect the transfusion port of the 1000 mL container to a blood warmer, a blood filter or both, and to the patient so that the blood is transferred back to the patient.

Blood flow rate will be such that time from collection to transfusion will be less than about 10 minutes, less than about 9 minutes, in less than about 8 minutes, less than about 7 minutes, less than about 6 minutes, less than about 5 minutes, less than about 4 minutes, or less than about 3 minutes.

### Example 3

Sterility of the inner chamber of the blood collection and autotransfusion container device will be assessed by sampling fluids brought into contact with the inner chamber under aseptic conditions. Precautions will be taken to avoid contamination. Media suitable for the testing of sterility includes fluid thioglycollate medium (FTM), which is primarily intended for culture of anerobic bacteria; however, it will also detect aerobic bacteria. Soya-bean casein digest medium (SCDM) is suitable for the culture of both fungi and aerobic bacteria.

Each lot of ready-prepared medium will be tested with the following suitable commercially available strains of organisms as a positive control:

| | |
|---|---|
| Aerobic bacteria | |
| Staphylococcus Aureus | ATCC 6538 |
| Bacillus subtilis | ATCC 6633 |
| Pseudomonas aeruginosa | ATCC 9027 |
| Anerobic bacterium | |
| Clostridium sporogenes | ATCC 19404 or 11437 |
| Fungi | |
| Candida albicans | ATCC 10231 |
| Aspergillus brasiliensis | ATCC 16404 |

Direct inoculation positive control. The USP Suitability Test will be used to confirm that each lot of growth media used in the sterility test procedure will support the growth of fewer than 100 viable microorganisms. If the media cannot support the growth of the indicator organisms, then the test fails. Secondly, a portion of each media lot will also be incubated and assessed for sterility according to the incubation parameters established by the method. If the media is found to be non-sterile, then the test fails.

A small number of viable microorganisms (100 CFU) will be added to the culture medium in triplicate. As a second positive control, a small number of viable microorganisms also will be added to a sterile blood product and aliquots of the blood product in triplicate then tested for microbial growth.

Negative control: aliquots of sterile saline and aliquots of a sterile blood bank blood product in triplicate will be separately added to the culture medium

USP Product flush sterility test: the blood collection and autotransfuser container will be flushed with a sterile fluid. The eluate will be membrane filtered and placed into FTM and SCDM for cultivation.

Blood sample test. Sterile blood will be collected into the blood collection and autotransfuser container under aseptic conditions. Small aliquots will be removed from the contents of the blood collection and autotransfuser container in triplicate and transferred to bottles containing culture medium (Tryptic Soy Broth (TSB) (VWR, Cat. No. 29446-184) bottle for aerobic bacteria growth assay; Fluid Thioglycollate Media (FTM) (VWR Cat. #29446-138) bottle for anaerobic bacteria growth assay) so that the volume of the test product is not more than 10% of the volume of the medium.

The media will be examined for macroscopic evidence of microbial growth at intervals during the incubation period (not more than 14 days). The media should be clear and transparent against a light source. Turbidity (cloudiness) of the medium is indicative of microbial growth. Once growth is detected, Gram positive and Gram negative bacteria will be detected by Gram Stain (Gram Stain Kit, VWR, Cat. NO. BB231401) to confirm that the turbidity is due to microorganisms. If no evidence of microbial growth is found, the product to be examined complies with the test for sterility. If evidence of microbial growth is found, the inner chamber of the blood collection and autotransfuser container does not comply with the test for sterility, unless it can be demonstrated that the test was invalid for causes unrelated to the container. If the test is declared to be invalid, it is repeated with the same number of units as in the original test. If no evidence of microbial growth is found in the repeat test, the inner chamber of the container complies with the test for sterility. If microbial growth is found in the repeat test, the inner chamber of the container does not comply with the test for sterility.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A blood collection and autotransfusion container device for rapid collection and transfusion of blood or blood products, comprising:
a blood container,
an infusion member,
and a transfusion member;
wherein the infusion member is connected to the blood container and the infusion member is configured to be in fluid communication with a subcutaneous drainage site on the subject and the blood container; and
wherein the transfusion member is connected to the blood container and the transfusion member is configured to be in fluid communication with the blood container and a subcutaneous transfusion site on the subject;
wherein the blood container device is configured with the following characteristics:
a volume of about 25 mL, about 30 mL, about 35 mL, about 40 mL, about 45 mL, about 50 mL, about 80 mL, about 85 mL, about 90 mL, about 95 mL, about 100 mL, about 200 mL, about 450 mL, about 500 mL, about 600 mL, about 700 mL, about 800 mL, about 900 mL, about 1000 mL, about 1100 mL, or about 1200 mL, inclusive,
a length between about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, and about 40 cm, inclusive,
a width between about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, and about 30 cm, inclusive, and
made of biocompatible, deformable, compressible material;
wherein the infusion member has an internal diameter between about 3.0 mm, about 3.5 mm, about 4.0 mm, about 4.5 mm, about 5.0 mm, about 5.5 mm, about 5.0 mm, about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm, inclusive;
wherein the transfusion member has an internal diameter between about 3.0 mm, about 3.5 mm, about 4.0 mm, about 4.5 mm, about 5.0 mm, about 5.5 mm, about 5.0 mm, about 6.5 mm, about 7.0 mm, about 7.5 mm, about 8.0 mm, about 8.5 mm, about 9.0 mm, about 9.5 mm, about 10.0 mm, about 10.5 mm, about 11.0 mm, about 11.5 mm, about 12.0 mm, about 12.5 mm, about 13.0 mm, about 13.5 mm, about 14.0 mm, about 14.5 mm, about 15.0 mm, about 15.5 mm, about 16.0 mm, about 16.5 mm, about 17.0 mm, about 17.5 mm, about 18.0 mm, about 18.5 mm, about 19.0 mm, about 19.5 mm, about 20.0 mm, about 20.5, about 21.0 mm, about 21.5 mm, about 22.0 mm, about 22.5 mm, about 23.0 mm, about 23.5 mm, about 24.0 mm, about 24.5 mm, and about 25.0 mm, inclusive; and
wherein the blood collection and autotransfusion container is effective to collect and transfuse one unit of blood in less than about 10 minutes, in less than about 9 minutes, in less than about 8 minutes, less than about 7 minutes, less than about 6 minutes, less than about 5 minutes, less than about 4 minutes, or less than about 3 minutes.

2. The device of claim 1 wherein the blood collection and autotransfusion container further comprises one or more tubing fittings configured to connect to and be in fluid communication with the blood container and the infusion member or the blood container and the transfusion member, wherein the one or more tubing fitting comprises one or more port members, one or more tube members, one or more membrane member, one or more connector/coupler members, one or more filter members, one or more valve members, and/or one or more penetrating members, and/or any combination thereof.

3. The device of claim 1, wherein the blood collection and autotransfusion container further comprises one or more tubing accessories configured to attach to the blood container, infusion member or transfusion member, wherein the tubing accessories comprises one or more hanging members, one or more cap members, and/or one or more clamp members and/or any combination thereof.

4. The device of claim 1, wherein the infusion member comprises
one or more tubing fittings comprising a port member and a membrane member, and one or more tubing accessories comprising a cap member; or
one or more tubing fittings comprising a tube member and a penetrating member, and one or more tubing accessories comprising a cap member and a clamp member.

5. The device of claim 1, wherein the transfusion member comprises
one or more tubing fittings comprising a port member and a membrane member, and one or more tubing accessories comprising a cap member; or
one or more tubing fittings comprising a tube member and a penetrating member, and one or more tubing accessories comprising a cap member and a clamp member.

6. The device of claim 1 wherein the infusion member is flexible, the transfusion member is flexible, or both.

7. The device of claim 1 wherein the infusion member is clampable, the transfusion member is clampable, or both.

8. A method for rapidly collecting accumulated blood from a subject in need thereof and rapidly transfusing blood or blood products to the subject in need thereof comprising.
a) providing a blood collection and autotransfusion container device comprising:
a blood container,
an infusion member,
and a transfusion member;
wherein the infusion member is connected to the blood container and the infusion member is configured to be in fluid communication with a subcutaneous drainage site on the subject and the blood container; and
wherein the transfusion member is connected to the blood container and the transfusion member is configured to be in fluid communication with the blood container and a subcutaneous transfusion site on the subject;
b) draining accumulated blood from the body cavity of a subject wherein the blood collection and autotransfusion container is connected to the subject via the infusion member;
c) collecting the blood in the blood collection and autotransfusion container by channeling the blood through the infusion member to a blood collection compartment in the blood container; and
d) transfusing the collected blood back to the subject by connecting the blood collection and autotransfusion container to the subcutaneous access site of the subject via the transfusion member; wherein the transfusion member is configured to be in fluid communication with a subcutaneous transfusion site on the subject and the blood collection compartment in the blood container.
wherein the blood collection and autotransfusion container is effective to collect and transfuse one unit of blood in less than about 10 minutes, in less than about 9 minutes, in less than about 8 minutes, less than about 7 minutes, less than about 6 minutes, less than about 5 minutes, less than about 4 minutes, or less than about 3 minutes.

9. The method according to claim 6 the step of providing a blood collection and autotransfusion container further comprising
providing one or more tubing fittings configured to connect to and be in fluid communication with the blood container, infusion member or transfusion member; and
providing one or more tubing accessories configured to attach to the blood container, infusion member or transfusion member;
wherein the one or more tubing fitting comprises one or more port members, one or more tube members, one or more membrane member, one or more connector/coupler members, one or more filter members, one or more valve members, and/or one or more penetrating members, and/or any combination thereof; and
wherein the one or more tubing accessories comprises one or more hanging members, one or more cap members, and/or one or more clamp members and/or any combination thereof.

10. The method according to claim 6, the step of draining accumulated blood from the body cavity of a subject comprising forcing the accumulated blood to be drained from a subject wherein the force comprises a gravitational force or a suction force;
wherein forcing the accumulated blood to be drained from a subject by a gravitational force comprises configuring the spatial arrangement of the blood collection and autotransfusion container to be at a lower height than the body cavity of the subject;
wherein forcing the accumulated blood to be drained from a subject by a suction force comprises connecting a suction tube with suction action to the blood container of the blood collection and autotransfusion container and activating the suction action;
or optionally providing a chest drainage device, and wherein forcing the accumulated blood to be drained from a subject by a gravitational force comprises configuring the spatial arrangement of the chest drainage device to be at a lower height than the body cavity of the subject and the blood container of the blood collection and autotransfusion container to be at a lower height than the chest drainage device;
or optionally providing a chest drainage device, wherein forcing the accumulated blood to be drained from a subject by a suction force comprises connecting a suction tube with suction action to the chest drainage device and activating the suction action.

11. The method according to claim 6, the step of collecting the blood comprising connecting the subcutaneous drainage site to the infusion member of the blood collection and autotransfusion container and forcing the blood to be channeled to the blood collection and autotransfusion container wherein the force comprises a gravitational force;
wherein forcing the blood to be channeled to the blood collection and autotransfusion container by gravitational force comprises arranging the spatial configuration of the blood container of the blood collection and autotransfusion device to be at a lower height than the blood.

12. The method according to claim 6, the step of transfusing the collected blood back to the subject comprising forcing the blood to be transfused to the subject through the transfusion member wherein the force comprises a compression force, gravitational force, or by a pressurized pump;
wherein forcing the blood to be transfused to the subject through the transfusion member by a compression force comprises providing a compression chamber attaching the blood container of the blood collection and autotransfusion container in the compression chamber with compression action and activating the compression action;
wherein forcing the blood to be transfused to the subject through the transfusion member by a gravitational force comprises configuring the spatial arrangement of the blood container to be at a higher height than the subcutaneous transfusion site on the subject; or wherein forcing the blood to be transfused to the subject through the transfusion member by a pressurized pump comprises providing a pressurized pump and attaching the pressurized pump to the blood container of the blood collection and autotransfusion container activating the pressurizing action.

13. The method according to claim 6, the step of transfusing the collected blood back to the subject comprising:
filtering the collected blood by connecting a blood filter;
warming the collected blood by connecting a blood warmer; and
releasing the excess gas in the collected blood by connecting to a gas vent;
wherein each of the blood filter, the blood warmer and the gas vent is in fluid communication with the transfusion member and the subcutaneous transfusion site on the subject.

14. A blood collection and autotransfusion container device for rapid collection and transfusion of blood or blood products, comprising:
a blood container,
a flexible infusion member,
and a flexible transfusion member;
wherein the infusion member is connected to the blood container and the infusion member is configured to be in fluid communication with a subcutaneous drainage site on the subject and the blood container; and
wherein the transfusion member is connected to the blood container and the transfusion member is configured to be in fluid communication with the blood container and a subcutaneous transfusion site on the subject;
wherein the blood container is configured with the following characteristics:
a volume of about 600 mL to about 1200 mL inclusive,
a length between about 20 cm to about 23 cm inclusive,
a width between about 12 cm to about 13 cm inclusive, and
made of biocompatible, deformable, compressible material;
wherein the infusion member is a tube member of an internal diameter between about 0.635 cm to about 0.95 cm inclusive, a length of about 22 to 23 cm, and further comprises a penetrating member, a cap member, and a clamp member;
wherein the transfusion member is a port member of an internal diameter between about 0.3175 to about 0.635 cm inclusive, and further comprises a membrane member;
wherein the device optionally further comprises a micron filter of about 170 microns to about 260 microns and is connected to at least one tube member and at least one penetration member, and is configured to be in fluid communication with the blood container and the infusion member;
wherein the device optionally further comprises a hanging member;
wherein the blood collection and autotransfusion container is effective to collect and transfuse one unit of blood in less than about 8 minutes.
